(19) United States
(12) Patent Application Publication
(10) Pub. No.: US 2026/0209787 A1
(43) Pub. Date: Jul. 23, 2026

(54) SYSTEMS AND METHODS FOR ENHANCING PROTEIN PRODUCTION

(71) Applicant: Duke University, Durham, NC (US)

(72) Inventors: Xinnian DONG, Durham, NC (US); Yezi XIANG, Durham, NC (US)

(21) Appl. No.: 19/136,572

(22) PCT Filed: Dec. 14, 2023
(86) PCT No.: PCT/US2023/083989

§ 371 Date: Jun. 6, 2025

Related U.S. Application Data
(60) Provisional application No. 63/432,775, filed on Dec. 15, 2022.

(51) Int. Cl. C12N 15/82; C12N 5/04; C12N 9/22; C12N 15/113; C12N 15/86

(52) U.S. Cl. CPC C12N 15/8279; C12N 5/04; C12N 9/222; C12N 15/113; C12N 15/8262; C12N 15/8274; C12N 15/86; C12N 2310/20; C12N 2310/531; C12N 2510/00; C12N 2800/107

(57) ABSTRACT

The present disclosure describes, in part, systems and methods for modulating or enhancing protein production.

### CROSS-REFERENCE TO RELATED APPLICATIONS

This application claims the benefit of U.S. Provisional Application No. 63/432,775, filed Dec. 15, 2022, which is incorporated herein by reference.

### STATEMENT REGARDING FEDERALLY SPONSORED RESEARCH OR DEVELOPMENT

This invention was made with Government support under Federal Grant nos. IOS-1645589 and IOS-2041378 awarded by the National Science Foundation, and under Federal Gran no. R35-GM118036-06 awarded by the National Institutes of Health National Institute of General Medical Sciences (NIH/NIGMS). The Federal Government has certain rights to this invention.

### SEQUENCE LISTING

The Sequence Listing written in file DU7883PCT_SeqListing_ST26.xml is 99 kilobytes in size, was created 12 Dec. 2023, and is hereby incorporated by reference.

### BACKGROUND

In eukaryotes, protein translation is normally cap-dependent. The m7G-cap of the mRNA is recognized by the 43S translation preinitiation complex comprised of the 40S ribosomal subunit and the eIF2-GTP-Met-tRNAi ternary complex. The preinitiation complex then scans along the 5′ leader sequence of the mRNA to initiate translation at a start codon. It has been proposed that ribosome scanning and start codon selection are regulated by elements in the 5′ leader sequence, such as RNA primary sequences (for example, the Kozak sequence context), upstream open reading frames (uORFs), secondary structures, and RNA modifications. Under stress conditions, such as nutrition depletion, hypoxia, or pathogen challenge, global translation is reprogrammed, leading to elevated stress-responsive protein production, but repressed growth-related protein synthesis. The mechanisms involved in the stress-induced translation have been investigated for a small number of key transcription factors (for example, yeast general control nondepressible 4 (GCN4) and mammalian activating transcription factor 4 (ATF4)), whose translation is normally inhibited by the uORFs in the 5′ leader sequences of their mRNAs. Upon stress, phosphorylation of eukaryotic Initiation Factor 2a (eIF2a) decreases the available ternary complex, resulting in reduced translation initiation from the start codons of uORFs (uAUGs) and prolonged scanning of the preinitiation complex to translate the downstream main open reading frames (mORFs) to promote cell survival. Recent global ribosome-sequencing (Ribo-seq, sequencing of ribosome-protected RNA fragments) studies have shown that uORFs are a prevalent feature in eukaryotic mRNAs.

Upstream open reading frames (uORFs), located before or overlap with the main coding ORF (mORF), can regulate cap-dependent translation efficiency in a transcript-specific manner. More than half of the human transcripts bear at least one uORF. A recent genome-wide ribosome-sequencing analysis revealed that uORFs act as regulators of both translation initiation and mRNA level. uORF-mediated translational control primarily regulates stress-responsive gene expression, which is important for cell-fate determination under stress. Under normal cellular conditions, uORFs suppress the translation of the downstream main coding sequence by 30-80%.

RNA helicases play a role in RNA metabolism, including transcription, translation, processing, and decay. These highly conserved enzymes use ATP to bind, unwind, and disrupt RNA structures and RNA-protein complexes. The two largest eukaryotic RNA helicase families are the DExD-box (DDX) and DExH-box (DHX) helicases, which share evolutionarily conserved motifs within their core.

eIF4A, a DEAD-box helicase, is a minimal DDX protein that is the best-characterized RNA helicase in translation initiation. Another conserved and essential DEAD-box RNA helicase, Ded1p, comes from yeast. How Ded1p and its orthologs engage RNAs in translation initiation has been a longstanding, unresolved question.

Recently, it was demonstrated that inactivation of the DEAD-box RNA helicase, Ded1p, in yeast leads to the accumulation of secondary structures in the 5′UTR of mRNAs, which are accompanied by translation initiation at near-cognate start codons located upstream of these structures, and decreased translation of the corresponding mORFs.

Experiments by Kozak's laboratory in the 1980s provided some of the earliest clues about how sequences and secondary structures surrounding the start codon can influence initiation. A recent genome-wide analysis showed that mRNA secondary structures are commonly present downstream from non-AUG start codons and from AUG codons in poor contexts, suggesting that secondary structure may be used to bolster translation initiation from weak initiation sites. It has been shown that recognition by mammalian ribosomes of an AUG codon in a suboptimal context, as well as that of non-AUG codons in general, is enhanced by the presence of a downstream stem-loop (hairpin) structure. This appears to be a common feature for many of the mRNAs whose translation has been shown to initiate at non-AUG codons—an effect presumably due to the slowing of scanning ribosomes and increased codon sampling time.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

Described are messenger RNAs (mRNAs) comprising: (a) an upstream open reading frame (uORF) comprising an upstream start codon, and a sequence that forms a stem loop structure operably linked to the upstream start codon, wherein the stem loop structure is about 1 to about 35 nucleotides downstream of the upstream start codon (corresponding to positions +4 to +38 (see Table 1), and wherein the stem loop structure comprises a stem having about 12 to about 20 base pairs; and (b) a heterologous open reading frame (ORF) encoding a polypeptide, wherein the uORF is 5′ of the heterologous ORF and is operably linked to the heterologous ORF; and wherein the uORF regulates translation of the heterologous ORF. In some embodiments, the upstream start codon comprises an upstream AUG (uAUG). In some embodiments, the stem has a folding energy of about −19.9 kcal mol⁻¹ to about −34.1 kcal mol⁻¹.

Described are messenger RNAs (mRNAs) comprising: (a) an upstream open reading frame (uORF) comprising an upstream start codon, and a sequence that forms a secondary structure operably linked to the upstream start codon, wherein the secondary structure begins about 1 to about 35 nucleotides downstream of the upstream start codon and has a folding energy of about −19.9 kcal mol⁻¹ to about −34.1 kcal mol⁻¹ when calculated for nucleotides +4 to +104 relative to the upstream start codon; and (b) a heterologous open reading frame (ORF) encoding a polypeptide, wherein the uORF is 5′ of the heterologous ORF and is operably linked to the heterologous ORF; and wherein the uORF regulates translation of the heterologous ORF. In some embodiments, the upstream start codon comprises an upstream AUG (uAUG). In some embodiments, the secondary structure comprises a stem loop structure. In some embodiments, the secondary structure comprises two or more stem structures.

In some embodiments, the stem loop structure starts at about position +9 to about position +26, about position +13 to about position +17, or about position +15. In some embodiments, the stem loop structure has a folding energy of about −19.9 kcal mol⁻¹ to about −34.1 kcal mol⁻¹, or −26.8±5 kcal mol⁻¹. In some embodiments, the percentage GC content of the stem of the stem loop structure is about 46% to about 61%, at least 50%, or is greater than the percentage GC content of the loop of the stem loop structure. In some embodiments, the percentage GC content of the loop of the stem loop structure is about 28% to about 50%, or less than 50%.

In some embodiments, the heterologous ORF encodes a protein selected from the group consisting of: a polypeptide that is a transcription factor; a reporter polypeptide; a polypeptide that confers resistance to drugs or agrichemicals; a polypeptide involved in resistance of plants to viral pathogens, bacterial pathogens, fungal pathogens, oomycete pathogens, phytoplasmas, or nematodes; and a polypeptide involved in the growth or development of plants. The heterologous ORF can be, but is not limited to, MLO, EDR1, Pi21, OsSWEET11, OsSWEET13, OsSWEET14, eIF4E, DMR6 (SlDMR6-1), Sr35, Sr50, Sr33, Pik1 and Pik2, RGA5 and RGA4, RRS1 and RPS4, RBS1, CsLOB1, PBS1, Xa27, MAPK3K StVIK1, COI1, IPA1, OsHEN1, SNC1, NPR1, CDP-DAG, RBL1. BBS1, HPL3, CSLF6, LIL1, RLIN1, SPL3 (OsEDR1 ACDR1), LMS, OsLOL1, MLO, OsSL, LRD6-6, SPL5, SPL7, SPL11, SPL18, SPL28, ACLA2, ABC1, SPL33, OsSPL35, SPL40, WSP1, X115, OsSSI2, GF14e, NOE1, EBR1, OsCUL3a, NLS1, OsRLR1, SNC1, SSI4, SLH1, CHS3-2D, CHS3-1, CHS2, UNI-1D, BAK1, BKK1, BIR1, SNC4-1D, CERK1-4, SNC2-1D, RIN4, CPR1, SRFR1, CPN1/BON1, MKP1, LSD1, ACD11, CPR5, MEKK1, MPK4, MKK1, MKK2, ACD6, BDA1-17, DND1, DND2/HLM1, CPR22, NPR3 NPR4, SR1/CAMTA3, PUB13, CPR6-1, SSI2, SYP121, SYP122, CAD1, or NSL1, and orthologs thereof.

In some embodiments, translation of the heterologous ORF is inducible. Translation of the heterologous ORF can be induced by a stress response, an immune response, a stress-induced helicase, an immune response-induced helicase, or an antisense oligonucleotide that specifically hybridizes to a sequence in the stem loop structure. The stress-induced helicase or the immune response-induced helicase can comprise: a RH37 helicase or an ortholog thereof, a RH11 helicase or an ortholog thereof, a RH52 helicase or an ortholog thereof, a Ded1p helicase or an ortholog thereof, or a DDX3X helicase or an ortholog thereof.

In some embodiments, any of the described mRNA can be encoded by a DNA molecule. In some embodiments, the DNA molecule further comprises a promoter sequence operably linked to the sequence encoding the mRNA. The promoter can be, but is not limited to, a plant promoter; a plant virus promoter; a promoter from a non-viral plant pathogen; a mammalian cell promoter; or a mammalian virus promoter.

In some embodiments, the DNA molecule further encodes a helicase, The helicase can be, but is not limited to, a RH37 helicase or an ortholog thereof, a RH11 helicase or an ortholog thereof, a RH52 helicase or an ortholog thereof, a Ded1p helicase or an ortholog thereof, or a DDX3X helicase or an ortholog thereof.

In some embodiments, vectors are described that comprise any of the described mRNAs or DNA molecules encoding any of the described mRNAs. The vector can be, but is not limited to, a viral vector, a transposon, a plasmid, or a CRISPR system.

In some embodiments, modified cells comprising any of the described mRNAs or DNA molecules encoding any of the described mRNAs are described. The cell can be, but is not limited to, a mammalian cell or a plant cell. Also described are plant propagation materials comprising one or more of the described modified cells.

Also described are plants comprising the described modified cells. In some embodiments, the plant is a modified or transgenic plant.

In some embodiments, DNA molecules are described that encoding mRNA comprising: (a) an upstream open reading frame (uORF) comprising an upstream start codon, and a sequence that forms a stem loop structure operably linked to the upstream start codon, wherein the stem loop structure is about 1 to about 35 nucleotides downstream of the upstream start codon, and wherein the stem loop structure comprises a stem having about 12 to about 20 base pairs; and (b) a heterologous sequence comprising a synthetic polylinker, a ligation independent cloning sequence, a sequence recognized by one or more restriction enzymes, or a heterologous open reading frame (hORF) encoding a polypeptide, wherein the uORF is 5′ of the heterologous sequence and is operably linked to the heterologous sequence. In some embodiments, the encoded mRNA comprises two or more uORFs and/or upstream start codons. In some embodiments, the stem has a folding energy of about −19.9 kcal mol⁻¹ to about −34.1 kcal mol⁻¹.

In some embodiments, DNA molecules are described that encoding mRNA comprising: (a) an upstream open reading frame (uORF) comprising an upstream start codon, and a sequence that forms a secondary structure operably linked to the upstream start codon, wherein the secondary structure begins about 1 to about 35 nucleotides downstream of the upstream start codon and has a folding energy of about −19.9 kcal mol⁻¹ to about −34.1 kcal mol⁻¹ when calculated for nucleotides +4 to +104 relative to the upstream start codon; and (b) a heterologous sequence comprising a synthetic polylinker, a ligation independent cloning sequence, a sequence recognized by one or more restriction enzymes, or a heterologous open reading frame (hORF) encoding a polypeptide, wherein the uORF is 5′ of the heterologous sequence and is operably linked to the heterologous sequence. In some embodiments, the encoded mRNA comprises two or more uORFs and/or upstream start codons. In some embodiments, the upstream start codon comprises an upstream AUG (uAUG). In some embodiments, the secondary structure comprises a stem loop structure. In some embodiments, the secondary structure comprises two or more stem structures.

In some embodiments, methods for generating a cell comprising an inducibly-expressed polypeptide are described. The methods comprise: introducing any of the described mRNAs, any or the described DNA molecules encoding any of the described mRNAs, or any of the described vectors into the cell.

In some embodiments, methods for generating a cell in which a polypeptide can be inducibly expressed are described. The methods comprise modifying an endogenous gene encoding the polypeptide in the cell to produce a modified gene, wherein the modified gene encodes an mRNA comprising, (a) a heterologous upstream open reading frame (uORF) comprising an upstream start codon, and a sequence that forms a stem loop structure operably linked to the upstream start codon, wherein the stem loop structure is about 1 to about 35 nucleotides downstream of the upstream start codon, and wherein the stem loop structure comprises a stem having about 12 to about 20 base pairs or a sequence that forms a secondary structure operably linked to the upstream start codon, wherein the secondary structure begins about 1 to about 35 nucleotides downstream of the upstream start codon and has a folding energy of about −19.9 kcal mol- to about −34.1 kcal mol⁻¹ when calculated for nucleotides +4 to +104 relative to the upstream start codon; and (b) an open reading frame (ORF) encoding the polypeptide, wherein the heterologous uORF is 5′ of the ORF and is operably linked to the ORF. In some embodiments, translation of the ORF is induced by a stress response, an immune response, stress-induced helicase, an immune response-induced helicase, or an antisense oligonucleotide that specifically hybridizes to a sequence in the stem loop structure. In some embodiments, the stress-induced helicase or the immune response-induced helicase comprises a RH37 helicase or an ortholog thereof, a RH11 helicase or an ortholog thereof, a RH52 helicase or an ortholog thereof, a Ded1p helicase or an ortholog thereof, or a DDX3X helicase or an ortholog thereof. In some embodiments, the methods further comprise expressing a heterologous helicase in the cell. The helicase can be, but is not limited to a RH37 helicase or an ortholog thereof, a RH11 helicase or an ortholog thereof, a RH52 helicase or an ortholog thereof, a Ded1p helicase or an ortholog thereof, or a DDX3X helicase or an ortholog thereof. In some embodiments, the methods further comprise contacting the cell with an antisense oligonucleotide that specifically hybridizes to a sequence in the stem loop structure.

Also described are messenger RNA (mRNAs) comprising: a start codon, and a sequence that forms a stem loop structure operably linked to the start codon, wherein the stem loop structure is about 1 to about 35 nucleotides downstream of the start codon, and wherein the stem loop structure comprises a stem having about 12 to about 20 base pairs.

In some embodiments, the stem loop structure starts at about position +9 to about position +26, about position +13 to about position +17, or about position +15. In some embodiments, the stem loop structure has a folding energy of about −19.9 kcal mol⁻¹ to about −34.1 kcal mol-1. In some embodiments, the percentage GC content of the stem of the stem loop structure is about 46% to about 61%, is at least 50%, or is less than the percentage GC content of the loop of the stem loop structure. In some embodiments, the percentage GC content of the loop of the stem loop structure is about 28% to about 50%, or less than 50%. In some embodiments, the polypeptide comprises a therapeutic protein.

Also described are messenger RNA (mRNAs) comprising: a start codon, and a sequence that forms a secondary structure operably linked to the start codon, wherein the secondary structure begins about 1 to about 35 nucleotides downstream of the start codon and has a folding energy of about −19.9 kcal mol⁻¹ to about −34.1 kcal mol⁻¹ when calculated for nucleotides +4 to +104 relative to the start codon and/or a folding energy of about −19.9 kcal mol⁻¹ to about −34.1 kcal mol⁻¹.

In some embodiments, DNA molecules comprising sequences encoding the mRNAs are provided. In some embodiments, the DNA molecule further comprises a promoter sequence operably linked to the sequence encoding the mRNA. In some embodiments, the promoter is selected from the group consisting of: a plant promoter; a plant virus promoter; a promoter from a non-viral plant pathogen; a mammalian cell promoter; and a mammalian virus promoter.

In some embodiments, vectors are described that comprise the mRNAs or the DNA molecules that encode the mRNAs. The vector can be, but is not limited to, a viral vector, a transposon, a plasmid, or a CRISPR system.

In some embodiments, modified cells are described that comprise the mRNAs or the DNA molecules encoding the mRNAs. The cell can be, but is not limited to, a mammalian cell or a plant cell.

In some embodiments, methods are described for increasing translation of an ORF in a cell. The methods comprise modifying a nucleic acid encoding the ORF to contain a stem loop structure, wherein the stem loop structure is operably linked to the start codon of the ORF, is about 1 to about 35 nucleotides downstream of the start codon, and comprises a stem having about 12 to about 20 base pairs. In some embodiments, modifying the nucleic acid encoding the ORF comprises substituting one or more codons downstream of the ORF thereby forming the stem loop structure. Substituting the one or more codons can be performed without altering the encoded amino acid sequence or by making one or more conservative amino acids changes to the coding sequence. In some embodiments, the stem has a folding energy of about −19.9 kcal mol⁻¹ to about −34.1 kcal mol⁻¹.

In some embodiments, methods are described for increasing translation of an ORF in a cell. The methods comprise modifying a nucleic acid encoding the ORF to contain a sequence that forms a secondary structure operably linked to a start codon of the ORF, wherein the secondary structure begins about 1 to about 35 nucleotides downstream of the start codon and has a folding energy of about −19.9 kcal mol⁻¹ to about −34.1 kcal mol⁻¹ when calculated for nucleotides +4 to +104 relative to the upstream start codon. In some embodiments, modifying the nucleic acid encoding the ORF comprises substituting one or more codons downstream of the ORF thereby forming the secondary structure. Substituting the one or more codons can be performed without altering the encoded amino acid sequence or by making one or more conservative amino acids changes to the coding sequence. In some embodiments, the secondary structure comprises a stem loop structure. In some embodiments, the secondary structure comprises two or more stem structures.

In some embodiments, methods are described for increasing translation of a gene having an upstream open reading frame comprising an upstream start codon and a sequence that forms a stem loop structure operably linked to the upstream start codon, wherein the stem loop structure is about 1 to about 35 nucleotides downstream of the upstream start codon. The methods comprise contacting a cell containing the gene with an antisense oligonucleotide that specifically hybridizes to a sequence in the stem loop structure.

Described are mRNA stem loop structures comprising sequences that form stem loop structures, wherein the stem loop structures comprise a stem having about 12 to about 20 base pairs and/or a folding energy of about −19.9 kcal mol⁻¹ to about −34.1 kcal mol⁻¹. When operably linked to a start codon, and positioned about 1 to about 35 nucleotides downstream of the start codon, the stem loop structures modulate translation initiation at the start codon. In some embodiments, the stem loop structures increase translation from a start codon. In some embodiments, the stem loop structures conditionally modify translation of the start codon. In some embodiments, the stem loop structures increase translation form the start codon in the absence of stress relative to translation from the start codon in the presence of stress. The start codon can be an upstream start codon (e.g., uAUG) or a main start codon (e.g., mAUG).

Also described are mRNAs comprising a sequence that forms a secondary structure operably linked to a start codon, wherein the secondary structure begins about 1 to about 35 nucleotides downstream of the start codon and has a folding energy of about −19.9 kcal mol⁻¹ to about −34.1 kcal mol⁻¹ when calculated for nucleotides +4 to +104 relative to the start codon. The secondary structures modulate translation initiation at the start codon. In some embodiments, the stem loop structures increase translation from a start codon. In some embodiments, the stem loop structures conditionally modify translation of the start codon. In some embodiments, the stem loop structures increase translation form the start codon in the absence of stress relative to translation from the start codon in the presence of stress. The start codon can be an upstream start codon (e.g., uAUG) or a main start codon (e.g., mAUG).

### BRIEF DESCRIPTION OF THE DRAWINGS

The accompanying Figures and Examples are provided by way of illustration and not by way of limitation. The foregoing aspects and other features of the disclosure are explained in the following description, taken in connection with the accompanying example figures (also “FIG.”) relating to one or more embodiments.

FIG. 1. Translational dynamics of uORF-containing transcripts. a, A volcano plot of global translational efficiency (TE) changes during pattern-triggered immunity. TE-up: transcripts with upregulated TE (p value<0.05, log₂ fold change>0.16); TE-nc: transcripts with no changes in TE (p value>0.05); TE-down: transcripts with downregulated TE (p value<0.05, log₂ fold change<−0.16). b, Number and percent of transcripts with translating uAUGs in the TE-up, TE-nc, and TE-down groups. Fisher's Exact Test was used to determine the p value of the difference between groups. c, A box plot of ribosome occupancy (normalized read counts) on translating uAUGs in the TE-up, TE-nc, and TE-down transcripts under the mock condition. Boxes represent the interquartile range (IQR), and whiskers indicate data within 1.5×IQR of the top (Q3) and bottom (Q1) quartiles. P values were calculated by two tailed Mann-Whitney tests. d, Histograms with density curves of log₂ fold change of ribosome occupancy on translating uAUGs in the TE-up, TE-nc, and TE-down transcripts in response to elf18 treatment. μ, averaged log₂ fold change value. P values were calculated by two tailed paired t tests. e, Ribosome occupancy on the uORF(s) in representative TE-up transcripts, namely TBF1, ZIK10, CAF1J, and ZF-MYND (AT1G70160.1), in response to mock and elf18 treatment. P values were calculated by two tailed student's t test. Values are means±SDs. Each dot represents a biological replicate.

FIG. 2. Global SHAPE-MaP and deep learning analysis reveal RNA secondary structures downstream of mAUGs and uAUGs in dictating translation initiation. a, Comparison of the Kozak sequence contexts (AG-content) flanking mAUGs and translating uAUGs. P values were calculated by Chi-Square test. b, Average SHAPE reactivities across all expressed transcripts aligned by start codons of CDS in the mock condition. Line corresponds to average reactivity for every three nucleotides. Ave.=average SHAPE reactivity across all of the nucleotides. Shading corresponds to 100 nt downstream of mAUG. c, Violin plots show the comparisons of SHAPE reactivities of the 50 nt upstream and the 50 nt downstream of mAUGs and uAUGs in different categories of transcripts under mock conditions. d, Boxplots show differences in SHAPE reactivities in the 50 nt upstream and the 50 nt downstream of translating uAUGs in representative TE-up transcripts. For transcripts with two translating uAUGs, the major inhibitory uAUGs (i.e., uAUG2 in TBF1 and ZIK10) are shown. e, Boxplots show the folding energy differences of the RNA secondary structure downstream of predicted initiating and non-initiating AUGs (including mAUGs and internal AUGs, left) and uAUGs (right). f, Distribution of the base-pair numbers in hairpin structures and folding energies of the RNA secondary structures downstream of predicted initiating AUGs. g, Heatmap shows the frequencies of the nucleotides in the loop (left) and the stem (right) of the RNA secondary structures downstream of predicted initiating AUGs. Numbers 1 to 25 show the position of each base pair, which were counted starting from the loop. h, Models of the RNA secondary structures downstream of uAUG2 (uAUG2-ds) of the TBF1 transcript and mAUG (mAUG-ds) of the ERECTA transcript. TBF1 and ERECTA sequences are provided in SEQ ID NOs: 5 ad 6, respectively. i, Boxplot shows the difference in ribosome occupancy on predicted initiating and non-initiating uAUGs. For all the box plots in the figure, boxes represent the interquartile range (IQR), and whiskers indicate data within 1.5×IQR of the top (Q3) and bottom (Q1) quartiles. P values were calculated by two tailed Mann-Whitney tests.

FIG. 3. RNA secondary structures downstream of uAUGs dynamically regulate translation. a, elf18-induced averaged SHAPE reactivity changes across nucleotide downstream of translating uAUGs in TE-up transcripts (upper (darker) line) or TE-nc and TE-down transcripts (lower (lighter) line). b, SHAPE reactivity changes across nucleotide downstream of translating uAUGs of representative TE-up transcripts under mock and elf18-induced conditions. For transcripts with two translating uAUGs, the major inhibitory uAUG (i.e., uAUG2 in TBF1 and ZIK10) are shown. Nucleotides with median to high SHAPE reactivities are marked in darker bars, and the ones with elf18-induced increases in SHAPE reactivities are highlighted with asterisks. c, In vivo SHAPE-MaP probing of TBF1-uAUG2-Δds (left) and the effects of disrupting the base-pairing downstream of uAUG2 (uAUG2-Δds) on translation (right). The mutated regions are not conserved in primary protein sequences. 5′ LS_{TBF1}, 5′ leader sequence of the TBF1 transcript. TBF1-F and TBF1-uAUG2-Δds-F are FLUC fused in-frame with uORF2. P values were calculated by two tailed student's t test. Values are means±SDs. d, Effects of uAUG and double-stranded RNA (dsRNA) structures on translation of the synthetic reporter. The introduction of the dsRNA changed the folding energy of the downstream region (101 nt) of uAUG from −9.8 kcal/mol to −23.6 kcal/mol without changing its length. Data were analyzed by two tailed student's t test. Values are means±SDs. e, Downstream double-stranded structure enhances uAUG inhibition on the mammalian ATF4 translation. 5′ LS_{ATF4}, 5′ leader sequence of the ATF4 transcript. ATF4-m2, uAUG2 mutated to AAG. ATF4-uAUG2-ds, the downstream region of uAUG2 substituted with an artificial hairpin. ATF4-F and ATF4-uAUG2-ds-F are FLUC fused in-frame with uORF2. P values were calculated by two tailed student's t test. Values are means±SDs. f, g, Translation of mammalian BRCA1 is regulated by uAUGs (f) and double-stranded RNA structures (g). 5′ LS_{BRCA1}, 5′ leader sequence of the BRCA1 transcript. P values were calculated by two tailed student's t test. Values are means±SDs. g, In vivo SHAPE-MaP analysis (left) and model (right) of the 50 nt upstream and the 50 nt downstream of uAUG2 (dn2) and uAUG3 (dn3) in the BRCA1 transcript. Boxes represent the interquartile range (IQR), and whiskers indicate data within 1.5×IQR of the top (Q3) and bottom (Q1) quartiles. Sequence shown in g is provided in SEQ ID NO: 7. P values were calculated by two tailed Mann-Whitney tests. For c-f, each dot represents a biological replicate.

FIG. 4. RNA helicases unwind RNA secondary structures downstream of uAUGs to alleviate repression on translation from mAUGs. a, A volcano plot of translational efficiency changes of 54 known Arabidopsis RNA helicases upon elf18 treatment. b, Translational responses of the 5′ leader sequences of RH37 (5′ LS_{RH37}) and RH11 (5′ LS_{RH11}) to elf18 induction. P values were calculated by two tailed student's t test. Values are means±SDs. c, Impact of Dex-induced expression of YFP-tagged RNA helicases (RH37, RH11) (bottom) in regulating translation of the 35S:TBF15′ leader sequence-FLUC 35S:RLUC dual-luciferase reporter (top). HA-tagged RLUC levels were detected as internal controls. P values were calculated by two tailed student's t test. Values are means±SDs. d, Impact of Dex-induced expression of YFP-tagged RH37 in regulating translation of the TUB7 synthetic reporters (top). P values were calculated by two tailed student's t test. Values are means±SDs. For b-d, each dot represents a biological replicate. e, Box plots of in planta SHAPE reactivity changes in the endogenous uAUG-ds regions of representative TE-up and TE-nc transcripts in wild type (WT) and the helicase mutant (rh37 rh52). For transcripts with two translating uAUGs (TBF1, ZIK10, ZIK6, and bZIP1), changes in uAUG2-ds are shown. Boxes represent the interquartile range (IQR), and whiskers indicate data within 1.5×IQR of the top (Q3) and bottom (Q1) quartiles. Data were analyzed by Wilcoxon signed rank tests. f, The elf18-induced protection against Psm ES4326 on WT and helicase mutants (n>12 biological replications). Bacterial growth was measured two days post inoculation and presented as means±s.e.m. P values were calculated by two-way ANOVA. The experiment was repeated twice with similar results. g, A model on RNA secondary structure-mediated translational regulation of uORF-containing transcripts during pattern-triggered immunity.

FIG. 5. Quality and reproducibility of RNA-seq and Ribo-seq data. a, BioAnalyzer profiles showed high quality of the Ribo-seq libraries. Apart from the internal standard sized at 35 bp and 10380 bp, a single peak at ~150 bp was present in all the libraries for mock and elf18 treatment in all three biological replicates (Reps 1-3). b, Length distribution of all reads from the Ribo-seq libraries. c, d, Correlations among the three replicates of RNA-seq (c) and Ribo-seq (d) data from mock- and elf18-treated samples. Data are shown as correlations of log₂(RPKM+1) for all the genes. r, Pearson correlation coefficient. e, Metagene analysis on the average read counts surrounding start and stop codons for reads at different lengths (top). P-site offsets were detected at the length of 13-15 nt surrounding start codons and at the length of 17-19 nt surrounding stop codons (bottom). 5′ LS, 5′ leader sequence. f, Power spectral density of normalized Ribo-seq read counts in the 300 nt window downstream of the start codon shows 3-nt periodicity. g, Total RNA-seq and Ribo-seq read distribution in 5′ LS, CDS, and 3′UTR. Boxes represent the interquartile range (IQR), and whiskers indicate data within 1.5×IQR of the top (Q3) and bottom (Q1) quartiles. Grey circles represent RPKM values for individual outlier transcripts. h, Metagene analysis across normalized transcript for Ribo-seq reads in all the mock and elf18-induced samples with the read length ranging from 24 nt to 35 nt. 5′ LS=5′ leader sequence. 3′ UTR=3′ untranslated region.

FIG. 6. Global analysis of translational dynamics and uAUG-containing transcripts. a, A flowchart of RNA-seq and Ribo-seq data analysis. b, Strategy for identification of translating mAUGs and uAUGs (see Methods for details). c, Dual-luciferase reporter study (top) of translational responses of the 5′ leader sequences of 20 TE-up transcripts to elf18 induction (bottom). FLUC reporter without the inserted test sequence was used as a negative control (Neg Ctl). P values were calculated by two-tailed Student's t-test. Values are mean±s.e.m. (n=5 biological replicates). d/e, Gene ontology (GO) analysis on the 1157 TE-up transcripts (d) and 1150 TE-down transcripts (e). The size of the dot represents the number of genes that fall into each group. The color of the dot represents adjusted p value.

FIG. 7. Quality and reproducibility of global and targeted in planta SHAPE-MaP. a, A flowchart of in planta SHAPE-MaP protocol. b, Comparison of Arabidopsis in vivo 18S rRNA secondary structure detected using the DMS-based method performed in a previous study and the SHAPE-MaP protocol used in this study. Nucleotides 32 to 518 (provided in SEQ ID NO: 8) of the 18S rRNA phylogenetic secondary structure are shown in the model and are color-coded with SHAPE reactivities generated in this study. c, Pearson correlation among the four SHAPE-MaP biological replicates (by transcript) under each treatment condition. Boxes represent the interquartile range (IQR), and whiskers indicate data within 1.5×IQR of the top (Q3) and bottom (Q1) quartiles. Circles represent Pearson correlation values for outliers. d, Cumulative fraction on the mutation rates of every nucleotide under each treatment condition.

FIG. 8. In vivo and in vitro SHAPE-MaP depict RNA structural features. a, Cumulative fraction on the SHAPE reactivities of nucleotides in 5′ leader sequence (5′ LS), CDS, and 3′UTR in mock- and elf18-treated samples. b, Average in vivo and in vitro SHAPE reactivities in the 5′ leader sequence (5′ LS), CDS and 3′ UTR across all expressed transcripts in the mock-treated samples aligned by the start and stop codons of CDS. Brown horizontal line marks the average in vivo SHAPE reactivity across all the nucleotides in mock-treated samples. c, Violin plots show the comparisons of in vivo and in vitro SHAPE reactivities of the 50 nt downstream regions of translating uAUGs in the TE-up transcripts and mAUGs in all expressed transcripts, as well as the 50 nt upstream region of stop codons in all expressed transcripts under the mock condition. d, Boxplot shows the difference in SHAPE reactivities in the 50 nt upstream and the 50 nt downstream of uAUG2s in the TBF1 and ZIK6 transcripts. Boxes represent the interquartile range (IQR), and whiskers indicate data within 1.5×IQR of the top (Q3) and bottom (Q1) quartiles. Circles represent values for outliers. Data were analyzed by two tailed Mann-Whitney tests.

FIG. 9. Deep learning on the SHAPE-MaP data supports a role of downstream double-stranded structures in dictating AUG selection for translation initiation. a, Flowchart of TISnet. The RNA secondary structures downstream of AUGs (AUG-ds) were predicted by RNAfold constrained by SHAPE reactivities. TISnet predicted the probability of initiating AUG by integrating the RNA primary sequence and secondary structure information of AUG-ds. AUGs with probability>0.9 are defined as predicted initiating AUGs; and AUGs with probability<0.9 are defined as predicted non-initiating AUGs. The sequence shown in a is provided in SEQ ID NO: 9. b, The input data and architecture of TISnet. The input data of TISnet include RNA sequences encoded by the one-hot encoding, and secondary structures encoded to 0 or 1. The TISnet architecture includes squeeze-excitation block, residual block (2D), and residual block (1D) adapted by the PrismNet model. c, The receiver operating characteristic (ROC) curves of the TISnet models trained with both the sequence and the structure information (top line), or solely with the sequence information (middle line), or solely with the structure information (bottom line). The AUC (area under the ROC curve) scores of three models are shown. d, Boxplot of the overall probabilities predicted by the TISnet model using downstream regions of mAUGs and internal AUGs as input data (left) or translating and non-translating uAUGs as input data (right). e, f, Examples of RNA structural models of downstream regions of predicted initiating AUGs (e) and non-initiating AUGs (f). The sequences shown in e are provided in, from left to right, SEQ ID NOs: 10, 11, and 12. The sequences shown in f are provided in, from left to right, SEQ ID NOs: 13, 14, and 15.

FIG. 10. Characterization of the class 1 AUG-ds. a, Pie plots show the percentage of different AUG-ds classes located in downstream regions of total predicted initiating AUGs, mAUGs, and translating uAUGs. b, The secondary structure models of mAUG-ds in the LRR1 transcript and uAUG2-ds in the ZF-MYND transcript. The sequences shown in b are provided in, from left to right, SEQ ID NOs: 16 and 17. c, The position weight matrix (PWM) of sequence motif of two stems and loop of the class 1 AUG-ds. d, Distribution of the distance between uAUG and the first nucleotide of the downstream hairpin element. Dashed lines represent the bottom (Q1), middle (Q2) and top (Q3) quartiles.

FIG. 11. uAUG-ds dynamically regulate translation in plants and mammalian cells. a, Overview of in vivo SHAPE reactivities across the 5′ leader sequences of TBF1 (top) and TBF1-uAUG2-Δds (bottom) expressed in N. benthamiana. Mutated uAUG-ds region is shaded. The sequences shown in a are provided in, from top to bottom, SEQ ID NOs: 18 and 19. b, DNA gel electrophoresis showing the 5′ RACE results of TBF1, TUB7 and their mutation variants (corresponding to FIG. 3c,d). c, Effects of different strengths of dsRNA structures on the translation of the synthetic reporter (no uAUG). The dsRNA structures were introduced without changing the length of 5′ leader sequences. Folding energies were calculated for the shaded region 54-153 nt downstream of the 5′ end. 5′ LSTUB7, the 5′ leader sequence of TUB7. Data were analyzed by two tailed Student's t-test. Different letters indicate statistically significant differences (P<0.05). Values are mean±s.d. (n=5 independent biological replicates). d, In-vitro-transcribed RNAs used in transfecting HEK293FT cells (corresponding to FIG. 3e,f and FIG. 11e,f). e, Translational regulatory activity of the Arabidopsis TBF1 5′ leader sequence (5′ LSTBF1) is maintained in HEK293FT cells. Mutagenesis of the 5′ leader sequence of TBF1 showed that, in HEK293FT cells, as in Arabidopsis, the double-stranded structure downstream of uAUG2 is required for inhibiting the reporter translation (top) by enhancing translation initiation from uAUG2 (bottom). TBF1-F and TBF1-uAUG2-Δds-F are FLUC fused in-frame with the first 66 nt of uORF2 (uORF2*). P values were calculated by two-tailed Student's t-test. Values are mean±s.d. (n=4 independent biological replicates). f, Effects of uAUG and RNA double-stranded structures on the synthetic reporter translation in HEK293FT cells. Data were analyzed by two-tailed Student's t-test. Values are mean±s.d. (n=4 independent biological replicates). In c,e,f, each dot represents a biological replicate. g, Violin plot comparisons of SHAPE reactivities in the 50 nt upstream and the 50 nt downstream of mAUGs in the TE-up transcripts in response to elf18 treatment. Boxes represent the interquartile range (IQR), and whiskers indicate data within 1.5×IQR of the top (Q3) and bottom (Q1) quartiles. Circles represent values for outliers. Data were analyzed by two tailed Mann-Whitney tests.

FIG. 12. Structural similarities of Arabidopsis homologous RNA helicases RH11, RH37 and RH52 with yeast Ded1p and mammalian DDX3X. a, Protein sequence alignment of Arabidopsis RH11 (SEQ ID NO: 24), RH37 (SEQ ID NO: 22), and RH52 (SEQ ID NO: 23) with their homologues in five other angiosperm species: Amborella trichopoda (Atrichopoda) (SEQ ID NOs: 36 and 37), Zea mays (Zmays) (SEQ ID NOs: 33, 34, and 35), Oryza sativa (Osativa) (SEQ ID NOs: 30, 31, and 32), Solanum lycopersicum (Slycopersicum) (SEQ ID NOs: 27 and 29), Medicago truncatula (Mtruncatula) (SEQ ID NOs: 25, 26, and 27), together with yeast Ded1p (SEQ ID NO: 21), human DDX3X (SEQ ID NO:20), as well as Arabidopsis eIF4A homologues (SEQ ID NOs: 38, 39, and 40). Numbers followed each name are PACIDs. ESPript 3.0 was used for protein sequence alignment visualization. Human DDX3X structure elements were used as references. Listed sequences from top to bottom are: SEQ ID NOs: 20, 21, 22, 23, 24, 25, 26, 27, 28, 29, 30, 31, 32, 33, 34, 35, 36, 27, 38, 39, and 40. b, Domain conservation of Arabidopsis RH11, RH37, RH52, eIF4A1, eIF4A2, and eIF4A3 with DDX3X/Ded1p regarding the nine sequence motifs (in the boxes and are illustrated from N terminus to C terminus). g, Conserved domains are indicated with asterisks. c, Pairwise alignment of yeast Ded1p with Arabidopsis RH11, RH37, and RH52 (c) and with Arabidopsis eIF4A1 and eIF4A2 (d) shows that RH11, RH37 and RH52, but not eIF4A1 and eIF4A2, are structurally similar to Ded1p. Protein structures were predicted by AlphaFold, and superimposed and visualized by PyMol v1.3.

FIG. 13. Genotyping of the helicase mutants. a-c, Schematics of CRISPR experiments and the Sanger sequencing results from rh37 (SEQ ID NO: 41) and rh52 (SEQ ID NO: 42) (a), rh11 (SEQ ID NOs: 43 and 44) and rh52 (SEQ ID NO: 45) (b), and rh11 (SEQ ID NO: 46) and rh52-2 (SEQ ID NO: 47) (c) double mutants. The short line with a darker end indicates guide RNA with the PAM sequence (darker end). d, Representative morphology of WT, eft, rh37 rh52, rh11 rh52, and rh11 rh52-2 plants prior to the elf18-induced protection assay. Higher order mutants rh37 rh11^{+/-} rh52, rh37^{+/-} rh11 rh52, and rh37^{+/-} rh11 rh52-2 are included in the photo to show their growth defect. e, Western blotting shows that the helicase double mutant (rh37 rh52) specifically compromises the elf18-mediated increases in protein levels from translating uAUG-containing transcripts (ARF2 and CH1), but not from transcripts without translating uAUGs (RBOHD and ICS1). The relative band intensity of the immunoblot (represented by numbers below the blot) was normalized to mock for each background. The experiment was repeated twice with similar results.

FIG. 14. Proposed mechanism for translational regulation of non-uAUG-containing transcripts. a, Percentage comparison of translating uAUG-containing, non-uAUG-containing and all transcripts with increased or decreased translation efficiency after elf18 induction (TE-up or TE-down). TE-up: transcripts with upregulated TE (P value<0.05, log₂-transformed fold change>0.16); TE-down: transcripts with downregulated TE (P value<0.05, log₂-transformed fold change<−0.16). b, GO enrichment analysis on the non-uAUG-containing transcripts. c, A proposed model of mAUG-ds-mediated translational regulation of non-uAUG-containing transcripts during PTI.

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e., at least one) of the grammatical object of the article. By way of example, “an element” means at least one element and can include more than one element.

In general, the term “about” indicates insubstantial variation in a quantity of a component of a composition not having any significant effect on the activity or stability of the composition. When the specification discloses a specific value for a parameter, the specification should be understood as alternatively disclosing the parameter at “about” that value. Also, the use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings. To the extent that any material incorporated by reference is inconsistent with the express content of this disclosure, the express content controls.

The use herein of the terms “including,” “comprising,” or ““having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations were interpreted in the alternative (“or”).

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. All ranges are to be interpreted as encompassing the endpoints in the absence of express exclusions, such as “not including the endpoints”; thus, for example, “within 10-15” includes the values 10 and 15. One skilled in the art will understand that the recited ranges include the end values, whole numbers in between the end values, and where practical, rational numbers within the range (e.g., the range 5-10 includes 5, 6, 7, 8, 9, and 10, and where practical, values such as 6.8, 9.35, etc.). When values are expressed as approximations, by use of the antecedent “about,” it will be understood that the particular value forms a further aspect. For example, if the value “about 10” is disclosed, then “10” is also disclosed.

A “heterologous” sequence is a sequence which is not normally present in a cell, genome, or gene in the genetic context in which the sequence is currently found. A heterologous sequence can be a sequence derived from the same gene (e.g., a different allele) and/or cell type, but introduced into the cell or a similar cell in a different context, such as on an expression vector or in a different chromosomal location or with a different promoter. A heterologous sequence can be a sequence derived from a different gene or species than a reference gene or species. A heterologous sequence can be from a homologous gene from a different species, from a different gene in the same species, or from a different gene from a different species. For example, a ORF sequence may be heterologous to an uORF in that it is not naturally linked to the uORF.

A “promoter” is a DNA regulatory region capable of binding an RNA polymerase in a cell (e.g., directly or through other promoter-bound proteins or substances) and initiating transcription of a coding sequence. A promoter may comprise one or more additional regions or elements that influence transcription initiation rate, including, but not limited to, enhancers. A promoter can be, but is not limited to, a constitutively active promoter, a conditional promoter, an inducible promoter, or a cell-type specific promoter.

“Operable linkage” or being “operably linked” refers to the juxtaposition of two or more components (e.g., a uORF and polypeptide coding sequence or a stem sloop sequence and a start codon) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

“Orthologs” are genes and products thereof in different species that evolved from a common ancestral gene by speciation and retain the same or similar function. An ortholog is a gene that is related by vertical descent and is responsible for substantially the same or identical functions in different organisms. For example, an A. thaliana NPR1 gene and Oryza sativa (rice) NH1 can be considered orthologs. Genes may share sequence similarity of sufficient amount to indicate they are orthologs. Protein may share three-dimensional structure of sufficient amount to indicate the proteins and the genes encoding them are orthologs. Methods of identifying orthologs are known in the art.

Sequence identity can be determined by aligning sequences using algorithms, such as BESTFIT, FASTA, and TFASTA in the Wisconsin Genetics Software Package Release 7.0, Genetics Computer Group, 575 Science Dr., Madison, Wis.), using default gap parameters, or by inspection, and the best alignment (i.e., resulting in the highest percentage of sequence similarity over a comparison window). Percentage of sequence identity is calculated by comparing two optimally aligned sequences over a window of comparison, determining the number of positions at which the identical residues occurs in both sequences to yield the number of matched positions, dividing the number of matched positions by the total number of matched and mismatched positions not counting gaps in the window of comparison (i.e., the window size), and multiplying the result by 100 to yield the percentage of sequence identity. Unless otherwise indicated the window of comparison between two sequences is defined by the entire length of the shorter of the two sequences.

The term “conservative substitution” or “conservative mutation,” refers to an alteration that results in the substitution of an amino acid with another amino acid that can be categorized as having a similar feature. Examples of categories of conservative amino acid groups defined in this manner can include: a “charged/polar group” including Glu (Glutamic acid or E), Asp (Aspartic acid or D), Asn (Asparagine or N), Gln (Glutamine or Q), Lys (Lysine or K), Arg (Arginine or R), and His (Histidine or H); an “aromatic group” including Phe (Phenylalanine or F), Tyr (Tyrosine or Y), Tip (Tryptophan or W), and (Histidine or H); and an “aliphatic group” including Gly (Glycine or G), Ala (Alanine or A), Val (Valine or V), Leu (Leucine or L), He (Isoleucine or I), Met (Methionine or M), Ser (Serine or S), Thr (Threonine or T), and Cys (Cysteine or C). Within each group, subgroups can also be identified. For example, the group of charged or polar amino acids can be sub-divided into sub-groups including: a “positively-charged subgroup” comprising Lys, Arg and His; a “negatively-charged sub-group” comprising Glu and Asp; and a “polar sub-group” comprising Asn and Gln. In another example, the aromatic or cyclic group can be sub-divided into sub-groups including: a “nitrogen ring sub-group” comprising Pro, His and Trp; and a “phenyl sub-group” comprising Phe and Tyr. In another further example, the aliphatic group can be sub-divided into sub-groups, e.g., an “aliphatic non-polar sub-group” comprising Val, Leu, Gly, and Ala; and an “aliphatic slightly-polar sub-group” comprising Met, Ser, Thr, and Cys. Examples of categories of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free —OH can be maintained; and Gln for Asn or vice versa, such that a free —NH2 can be maintained. In some embodiments, hydrophobic amino acids are substituted for naturally occurring hydrophobic amino acids, e.g., in the active site, to preserve hydrophobicity.

The terms “treat,” “treatment,” and the like, mean the methods or steps taken to provide relief from or alleviation of the number, severity, and/or frequency of one or more symptoms of a disease or condition in a subject. Treating generally refers to obtaining a desired pharmacological and/or physiological effect. The effect can be, but does not necessarily have to be, prophylactic in terms of preventing or partially preventing a disease, symptom, or condition thereof. The effect can be therapeutic in terms of a partial or complete cure of a disease, condition, symptom, or adverse effect attributed to the disease, disorder, or condition. The term treatment can include: (a) preventing the disease from occurring in a subject who may be predisposed to the disease but has not yet been diagnosed as having it; (b) inhibiting the disease, i.e., arresting its development; and (c) relieving the disease, i.e., mitigating or ameliorating the disease and/or its symptoms or conditions. Treating can refer to both therapeutic treatment alone, prophylactic treatment alone, or both therapeutic and prophylactic treatment. Those in need of treatment (subjects in need thereof) can include those already with a disease, disorder, or condition or those in which the disease, disorder, or condition is to be prevented. Treating can include inhibiting the disease, disorder, or condition, e.g., impeding its progress; and relieving the disease, disorder, or condition, e.g., causing regression of the disease, disorder, and/or condition. Treating the disease, disorder, or condition can include ameliorating at least one symptom of the particular disease, disorder, or condition, even if the underlying pathophysiology is not affected, e.g., such as treating the symptom without affecting or removing an underlying cause of the symptom.

The term “effective amount” or “therapeutically effective amount” refers to an amount sufficient to effect beneficial or desirable biological and/or clinical results.

The term “plant” includes whole plants, plant organs (e.g., leaves, stems, flowers, roots, reproductive organs, embryos and parts thereof, etc.), seedlings, seeds and plant cells, and progeny thereof. The class of plants which can be used in the method of the invention is generally as broad as the class of higher plants amenable to transformation techniques, including angiosperms (monocotyledonous and dicotyledonous plants), as well as gymnosperms. It includes plants of a variety of ploidy levels, including polyploid, (e.g., diploid, haploid and hemizygous.

A “regenerant” is a plant produced from a plant tissue cell, such as a genetically modified plant tissue cell.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e., living organism, such as a patient). In some embodiments, the subject comprises a human who is undergoing treatment using a system and/or method as prescribed herein.

Unless otherwise defined, all technical terms used herein have the same meaning as commonly understood by one of ordinary skill in the art to which this disclosure belongs.

### I. OVERVIEW

To survive stress, eukaryotes selectively translate stress-related transcripts while inhibiting growth-associated protein production. How this translational reprogramming occurs under biotic stress has not been systematically studied. To identify common features shared by transcripts with stress-upregulated translation efficiency (TE-up), high-resolution ribosome-sequencing was performed in Arabidopsis during pattern-triggered immunity, and it was found that TE-up transcripts are enriched with upstream open reading frames (uORFs). Under non-stress conditions, start codons of these uORFs (uAUGs) have higher-than-background ribosomal association. Upon immune induction, there is an overall downshift in ribosome occupancy at uAUGs, accompanied by enhanced translation of main ORFs (mORFs). Using in planta nucleotide-resolution mRNA structurome probing, it was discovered that this stress-induced switch in translation is mediated by highly structured regions detected downstream of uAUGs in TE-up transcripts.

Without stress, these structures are responsible for uORF-mediated inhibition of mORF translation by slowing progression of the translation preinitiation complex to initiate translation from uAUGs, instead of mAUGs. In response to immune induction, uORF-inhibition is alleviated by three Ded1p/DDX3X homologous RNA helicases which unwind the RNA structures, allowing ribosomes to bypass the inhibitory uORFs and upregulate defense protein production. Conservation of the RNA helicases suggests that mRNA structurome remodeling is a general mechanism for stress-induced translation across kingdoms.

The discovery of the first example showing that eukaryotes had evolved a general feature in the mRNA structure, not in the sequence, in controlling translation initiation, indicates that protein synthesis can be enhanced by introducing a secondary structure downstream of the translation start codon through either in vitro molecular engineering or in vivo gene-editing.

Further details of the systems and methods disclosed herein are provided in the Example below. Another aspect of the present disclosure provides all that is described and illustrated herein.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

### II. INDUCIBLE GENE CONSTRUCTS

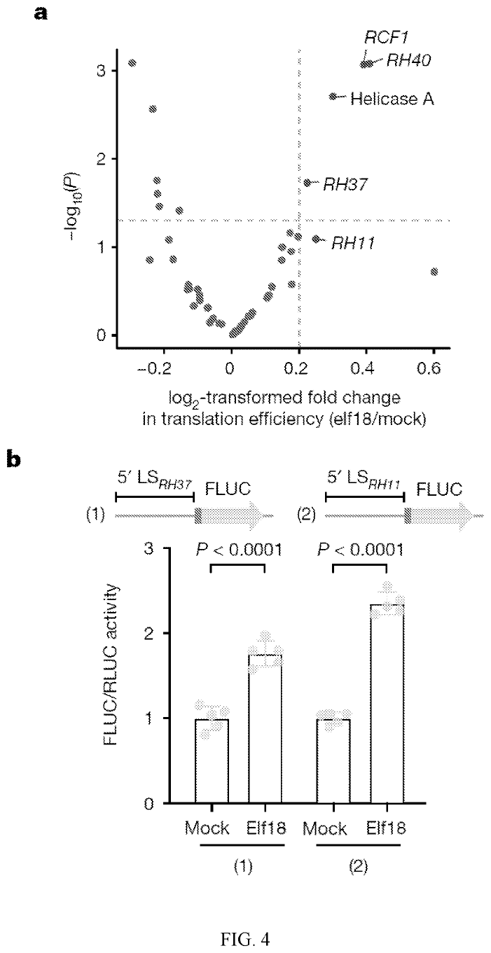

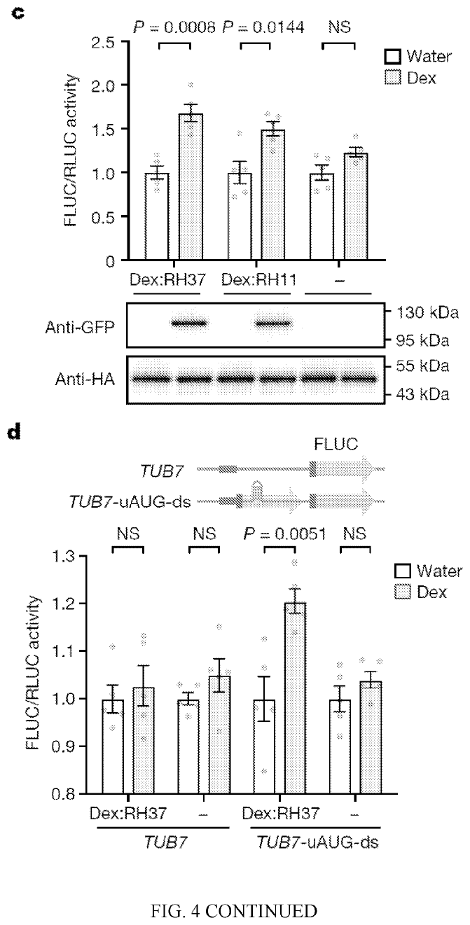

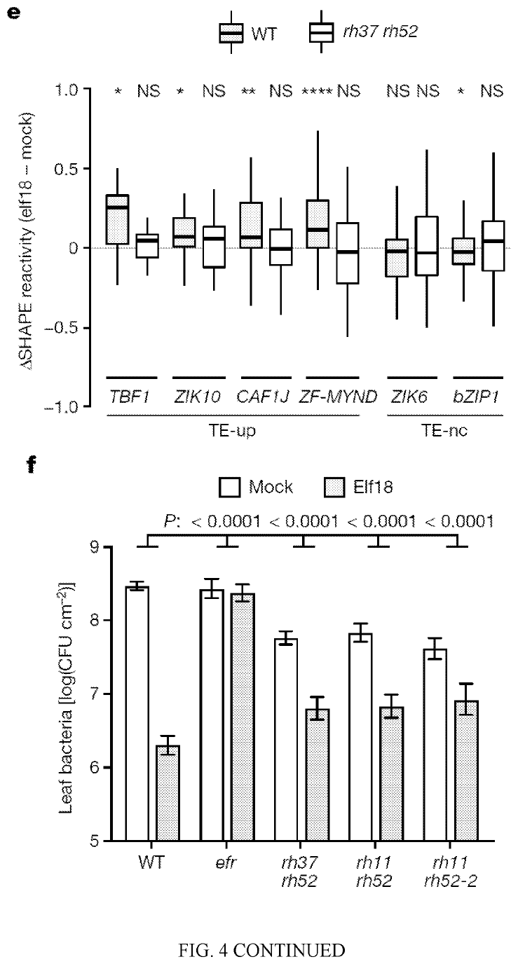

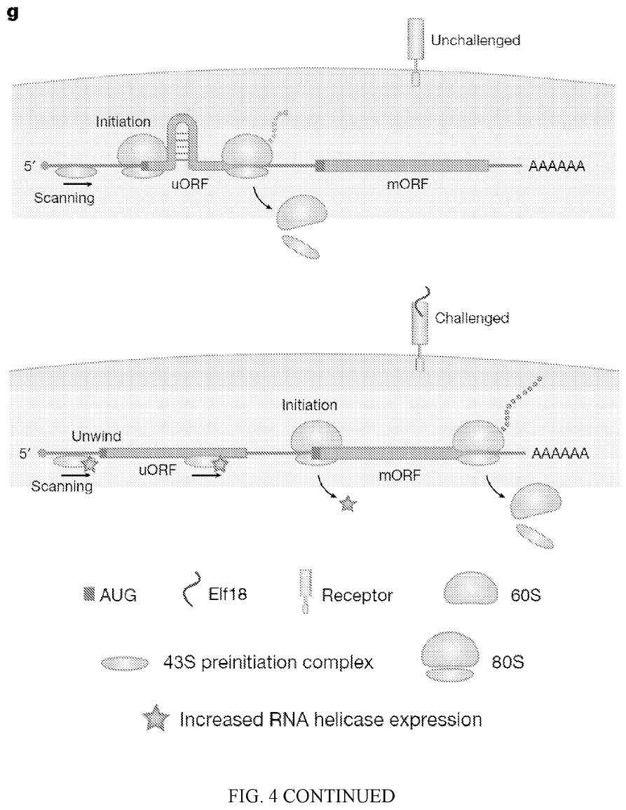

Described are expression constructs that provide for inducible expression of a polypeptide. The expression constructs can comprise: an upstream open reading frame (uORF) and a heterologous open reading frame (ORF) encoding the polypeptide, wherein the uORF comprises an upstream start codon and a sequence that forms a secondary structure operably linked to the upstream start codon. The secondary structure can begin, for example, about 1 to about 35 nucleotides downstream of the upstream start codon. In some embodiments, the secondary structure comprises a stem loop structure. The stem loop structure can comprise, for example, a stem having about 12 to about 20 base pairs (see FIG. 4, panel g, top panel). In some embodiments, the secondary structure comprises two or more base pairing regions (e.g., stems). In some embodiments, the secondary structure has a folding energy of about −19.9 kcal mol⁻¹ to about −34.1 kcal mol⁻¹ when calculated for nucleotides +4 to +104 relative to the upstream start codon. The uORF is 5′ of and operably linked to the ORF and regulates translation of the ORF. The uORF and the ORF are transcribed on a single mRNA. In some embodiments, for the downstream region (101 nt) of each AUG, RNA secondary structures and folding energy are predicted using RNAfold with SHAPE reactivity data used as a soft constraint involving a pseudo-free energy calculation under default parameters (the slope ‘m’ is 1.8 and the intercept is −0.6).

Also described are DNA molecules encoding a uORF and a heterologous sequence, wherein an mRNA encoded by the DNA molecule comprises the uORF and the heterologous sequence, wherein the uORF comprises an upstream start codon and a sequence that forms a secondary structure operably linked to the upstream start codon. The stem loop structure can be, for example, about 1 to about 35 nucleotides downstream of the upstream start codon. In some embodiments, the secondary structure comprises a stem loop structure. The stem loop structure can comprise, for example, a stem having about 12 to about 20 base pairs. In some embodiments, the secondary structure comprises two or more base pairing regions (e.g., stems). In some embodiments, the secondary structure has a folding energy of about −19.9 kcal mol¹ to about −34.1 kcal mol⁻¹ when calculated for nucleotides +4 to +104 relative to the upstream start codon. The uORF is 5′ of the heterologous sequence and is operably linked to the heterologous sequence. The heterologous sequence can be, but is not limited to, a synthetic polylinker, a ligation independent cloning sequence, a sequence recognized by a one or more restriction enzymes, or a heterologous open reading frame (hORF) encoding a polypeptide. A sequence encoding a polypeptide can be inserted into the synthetic polylinker, the ligation independent cloning sequence, or the sequence recognized by the one or more restriction enzymes.

The uORF start codon may be any codon known in the art that can use used as a start codon. In some embodiments, the uORF start codon is AUG (uAUG). In some embodiments, the uORF start codon is a non-uAUG start codon. A uORF non-AUG start codon can be, but is not limited to, CUG, GUG, ACG, UUG, AUU, AUC, AAG, AUA, or AGG.

The uORF start codon may be linked to, or present in the context of, a strong Kozak sequence, an average Kozak sequence, a weak Kozak sequence, or no identifiable Kozak sequence. A strong Kozak sequence indicates the nucleotide at position +4 (one nucleotide downstream of the start codon) is a consensus Kozak sequence nucleotide (e.g., a G) and the nucleotide at position −3 (three nucleotides upstream of the start codon) is a consensus Kozak sequence nucleotide (e.g., an A). An average Kozak sequence indicates that either the nucleotide at position +4 is a consensus Kozak sequence nucleotide or the nucleotide at position −3 is a consensus Kozak sequence nucleotide (e.g., an A), but not both. A weak Kozak sequence indicates that neither the nucleotide at position +4 nor the nucleotide at position and −3 is a consensus Kozak sequence nucleotide. A Kozak sequence can be, but is not limited to, (A/G)cc[start codon]G, wherein the start codon corresponds to positions +1, +2, and +3 (see Table 1).

In some embodiments, the stem loop structure of the uORF comprises a first stem sequence, a loop sequence, and a second stem sequence. The first and second stem sequences can be about 12 to about 24 nucleotides in length. In some embodiments, the stem of the stem loop structure of the uORF comprises about 12 to about 20 base pairs and starts about 1 to about 35 nucleotides downstream of the uORF start codon (i.e., the first nucleotide of the stem is located at about position +4 to about +38), wherein the first nucleotide of the upstream start codon is +1. The about 12 to about 20 base pairs in the stem can be contiguous or discontiguous. If discontiguous, the stem can comprise 12 to 20 base pairs (24 to 40 paired nucleotides) and about 1 to about 10 unpaired nucleotides. In some embodiments, the stem of the stem loop structure of the uORF contains no unpaired or mismatched nucleotides. In some embodiments, the stem of the stem loop structure of the uORF contains at least one unpaired or mismatched nucleotide. In some embodiments, the stem of the stem loop structure of the uORF contains 12, 13, 14, 15, 16, 17, 18, 19, or 20 base pairs. In some embodiments, the stem of the stem loop structure of the uORF contains no more than 10, no more than 9, no more than 8, no more than 7, no more than 6, no more than 5, no more than 4, no more than 3, no more than 2, or no more than 1 unpaired or mismatched nucleotides. In some embodiments, the stem of the stem loop structure of the uORF comprises about 12 to about 20 base pairs and/or has a folding energy of (AG) about −19.9 kcal mol⁻¹ to about −34.1 kcal mol⁻¹. In some embodiments, the stem of the stem loop structure of the uORF comprises about 12 to about 20 base pairs and has a folding energy of −27±7 kcal mol-1. In some embodiments, the stem of the stem loop structure of the uORF comprises about 12 to about 20 base pairs and has a folding energy of −27±6 kcal mol⁻¹. In some embodiments, the stem of the stem loop structure of the uORF comprises about 12 to about 20 base pairs and has a folding energy of about −26.8±5 kcal mol⁻¹. In some embodiments, the stem of the stem loop structure of the uORF comprises about 12 to about 20 base pairs, wherein the percentage GC content of the stem of the stem loop structure is about 46% to about 61%. In some embodiments, the percentage GC content of the stem of the stem loop structure is at least 50%. In some embodiments, the percentage GC content of the stem of the stem loop structure is greater than the percentage GC content of the loop of the stem loop structure. In some embodiments, the percentage GC content of the loop of the stem loop structure is about 28% to about 50%. In some embodiments, the percentage GC content of the loop of the stem loop structure is less than 50%. In some embodiments, the loop of the stem loop structure comprises about 1 to about 10 nucleotides. In some embodiments, the loop of the stem loop structure comprises about 3 to about 7 nucleotides. In some embodiments, the loop of the stem loop structure comprises 1, 2, 3, 4, 5, 6, 7, 8, 9, or 10 nucleotides. In some embodiments, the loop of the stem loop structure comprises the nucleotide sequence UCU or CUG. In some embodiments, the loop of the stem loop structure comprises the nucleotide sequence UCAGAUC. In some embodiments, the loop of the stem loop structure comprises at least 3, at least 4, at least 5, or at least 6 contiguous nucleotides from the sequence UCAGAUC.

ΔG can be calculated using methods available in the art to calculated folding energy of RNA secondary structure. ΔG can be determined experimentally using methods available in the art to measuring folding energy of RNA secondary structure. In some embodiments, for the downstream region (101 nt) of each AUG, RNA secondary structures and folding energy are predicted using RNAfold with SHAPE reactivity data used as a soft constraint involving a pseudo-free energy calculation under default parameters (the slope ‘m’ is 1.8 and the intercept is −0.6).

The stem of the stem loop structure of the uORF can comprise the nucleotide sequence of any of the stem structures disclosure herein, provided the stem contains 12 to 20 base pairs and/or has a folding energy of about −19.9 kcal mol⁻¹ to about −34.1 kcal mol¹. The stem loop structure of the uORF can comprise the nucleotide sequence of any of the stem loop structures disclosure herein, provide the stem contains 12 to 20 base pairs and/or has a folding energy of about −19.9 kcal mol- to about −34.1 kcal mol¹. In some embodiments, the stem loop structure comprises GACGTCGGTTCCGACGTC (SEQ ID NO: 73).

In some embodiments, the first nucleotide of the secondary structure or the stem is located at position +4, +5, +6, +7, +8, +9, +10, +11, +12, +13, +14, +15, +16, +17, +18, +19, +20, +21, +22, +23, +24, +25, +26, +27, +28, +29, +30, +31, +32, +33, +34, +35, +36, +37, or +38 relative to the first nucleotide of the upstream start codon. In some embodiments, the first paired nucleotide of the secondary structure or the stem is at about position +9 to about position +26 relative to the first nucleotide of the upstream start codon. In some embodiments, the first paired nucleotide of the secondary structure or the stem is at a about position +13 to about position +20 relative to the first nucleotide of the upstream start codon. In some embodiments, the first paired nucleotide of the secondary structure or the stem is at about position +13 to about position +17 relative to the first nucleotide of the upstream start codon. In some embodiments, the first paired nucleotide of the secondary structure or the stem is at about position +15 relative to the first nucleotide of the upstream start codon. In some embodiments, the secondary structure of the uORF is positioned such that a ribosome, or ribosome subunit, scanning the 5′ UTR of an mRNA containing the uORF pauses with the P site of the ribosome at or in proximity of the uORF start codon, thereby resulting in increased initiation of translation of the uORF. In some embodiments, the stem loop structure of the uORF is positioned such that a ribosome, or ribosome subunit, scanning the 5′ UTR of an mRNA containing the uORF pauses with the P site of the ribosome at or in proximity of the uORF start codon, thereby resulting in increased initiation of translation of the uORF.

TABLE 1
uORF (with AUG start codon) nucleotide numbering:

| nucl. | . . . | N | N | N | A | U | G | N | N | N | N | N | N | N | N | N | N | N | N | N | . . . |
|---|---|---|---|---|---|---|---|---|---|---|---|---|---|---|---|---|---|---|---|---|---|
| position | . . . | −3 | −2 | −1 | +1 | +2 | +3 | +4 | +5 | +6 | +7 | +8 | +9 | +10 | +11 | +12 | +13 | +14 | +15 | +16 | . . . |

1 nucleotide downstream of the start codon corresponds to position +4

In some embodiments, the expression construct encodes a mRNA comprising: a uORF operably linked 5′ to a heterologous ORF, wherein the uORF comprises a uAUG and a sequence that forms a stem loop structure operably linked to the uAUG. In some embodiments, the stem loop structure begins about 1 to about 35 nucleotides downstream of the upstream start codon. In some embodiments, the stem loop structure comprises a stem having about 12 to about 20 base pairs. In some embodiments, the stem loop structure has a folding energy of about −19.9 kcal mol-1 to about −34.1 kcal mol⁻¹.

In some embodiments, the expression construct encodes a mRNA comprising: a uORF operably linked 5′ to a heterologous ORF, wherein the uORF comprises a uAUG and a sequence that forms a secondary structure operably linked to the uAUG, wherein the secondary structure (a) begins about 1 to about 35 nucleotides downstream of the upstream start codon, (b) has a folding energy of about −19.9 kcal mol⁻¹ to about −34.1 kcal mol⁻¹ when calculated for nucleotides +4 to +104 relative to the upstream start codon. In some embodiments, the secondary structure comprises a stem loop structure. In some embodiments, the secondary structure comprises two or more stem structures pr base pairing regions.

In some embodiments, the expression construct comprises an mRNA. In some embodiments, the described expression constructs comprise a DNA molecule encoding the mRNA. DNA construct can further include a promoter sequence operably linked to the sequence encoding the mRNA. The promoter sequence can be any sequence known in the art for driving expression (i.e., transcription) of mRNA. The promoter can be, but is not limited to, a plant promoter, a plant virus promoter, a promoter from a non-viral plant pathogen, a mammalian cell promoter, a mammalian virus promoter, or an insect promoter.

In some embodiments, the DNA further encodes a helicase. The helicase can be, but is not limited to, a RH37 helicase or an ortholog thereof, a RH11 helicase or an ortholog thereof, a RH52 helicase or an ortholog thereof, a Ded1p helicase or an ortholog thereof, or a DDX3X helicase or an ortholog thereof.

The mRNA or DNA molecule encoding the mRNA can be provided on a vector. The vector can be, but is not limited to, a viral vector, a transposon, or a plasmid. The vector can be any vector known in the art for expressing a nucleic acid sequence in a cell (e.g., a plant cell or a mammalian cell). The vector can encode a CRISPR system or be a component of a CRISPR system.

Also described are cells comprising any of the described expression constructs, mRNAs, DNAs, or vectors. The cell can be, but is not limited to, a plant cell or a mammalian cell. The plant cell can be in a plant, a plant part, or a plant propagation material. The plant can be a transgenic plant.

In some embodiments, the described expression constructs provide for inducible expression of the polypeptide in a cell. In some embodiments, expression of the polypeptide is induced by a stress response in the cell. In some embodiments, translation of the heterologous ORF is increased by a stress response in the cell.

In some embodiments, expression of the polypeptide is induced by an immune response in the cell. In some embodiments, translation of the heterologous ORF is increased by an immune response in the cell.

In some embodiments, expression of the polypeptide is induced by a stress-induced helicase in the cell. In some embodiments, translation of the heterologous ORF is increased by a stress-induced helicase in the cell. The stress-induced helicase can be an endogenous helicase expressed by the cell or a heterologous helicase. The helicase can be, but is not limited to, a RH37 helicase or an ortholog thereof, a RH11 helicase or an ortholog thereof, a RH52 helicase or an ortholog thereof, or a Ded1p helicase or an ortholog thereof.

In some embodiments, expression of the polypeptide is induced by an immune-induced helicase in the cell. In some embodiments, translation of the heterologous ORF is increased by an immune-induced helicase in the cell. The immune-induced helicase can be an endogenous helicase expressed by the cell or a heterologous helicase. The helicase can be, but is not limited to, a DDX3X helicase or an ortholog thereof, or a DEAD-box family helicase or an ortholog thereof.

In some embodiments, expression of the polypeptide is induced by an antisense oligonucleotide (ASO) that specifically hybridizes to a sequence in the secondary (e.g., stem loop) structure. In some embodiments, translation of the heterologous ORF is increased by an ASO that specifically hybridizes to a sequence in the secondary structure. In some embodiments, translation of the heterologous ORF is increased by an ASO that specifically hybridizes to a sequence in the stem loop structure. The ASO can be delivered to the cell to increase expression of the polypeptide (i.e., increase translation of the heterologous ORF). The ASO specifically hybridizes to all or a portion of the first stem sequence, all or a portion of the second stem sequence, or a combination thereof. The ASO hybridizes to the stem loop sequence with sufficient affinity to disrupt formation of the stem loop structure.

Also described are mRNAs, or DNAs encoding the mRNAs, for expression a polypeptide in a cell, the mRNA comprising a start codon and a sequence that forms a stem loop structure operably linked to the start codon. In some embodiments, the stem loop structure is about 1 to about 35 nucleotides downstream of the start codon. In some embodiments, the stem loop structure comprises a stem having about 12 to about 20 base pairs.

Also described are mRNAs, or DNAs encoding the mRNAs, for expression a polypeptide in a cell, the mRNA comprising a start codon and a sequence that forms a secondary structure operably linked to the start codon. In some embodiments, the secondary structure begins is about 1 to about 35 nucleotides downstream of the start codon. In some embodiments, the secondary structure has a folding energy of about −19.9 kcal mol⁻¹ to about −34.1 kcal mol⁻¹ when calculated for nucleotides +4 to +104 relative to the start codon.

In some embodiments, the stem loop structure comprises a first stem sequence, a loop sequence, and a second stem sequence. The first and second stem sequences can be, for example, about 12 to about 24 nucleotides in length. In some embodiments, the stem of the stem loop structure comprises about 12 to about 20 base pairs and starts at about 1 to about 35 nucleotides downstream of the start codon (i.e., the first nucleotide of the stem is located at about position +4 to about +38), wherein the first nucleotide of the start codon is +1. The about 12 to about 20 base pairs in the stem can be contiguous or discontiguous. If discontiguous, the stem can comprise 12 to 20 base pairs (24 to 40 paired nucleotides) and about 1 to about 10 unpaired nucleotides. In some embodiments, the stem of the stem loop structure contains no unpaired or mismatched nucleotides. In some embodiments, the stem of the stem loop structure contains at least one unpaired or mismatched nucleotide. In some embodiments, the stem of the stem loop structure contains 12, 13, 14, 15, 16, 17, 18, 19, or 20 base pairs. In some embodiments, the stem of the stem loop structure contains no more than 10, no more than 9, no more than 8, no more than 7, no more than 6, no more than 5, no more than 4, no more than 3, no more than 2, or no more than 1 unpaired or mismatched nucleotides. In some embodiments, the stem of the stem loop structure comprises about 12 to about 20 base pairs and/or has a folding energy of (ΔG) about −19.9 kcal mol⁻¹ to about −34.1 kcal mol⁻¹. In some embodiments, the stem of the stem loop structure comprises about 12 to about 20 base pairs and has a folding energy of −27±7 kcal mol⁻¹. In some embodiments, the stem of the stem loop structure comprises about 12 to about 20 base pairs and has a folding energy of −27±6 kcal mol⁻¹. In some embodiments, the stem of the stem loop structure comprises about 12 to about 20 base pairs and has a folding energy of about −26.8±5 kcal mol-1. In some embodiments, the stem of the stem loop structure comprises about 12 to about 20 base pairs, wherein the percentage GC content of the stem of the stem loop structure is about 46% to about 61%. In some embodiments, the percentage GC content of the stem of the stem loop structure is at least 50%. In some embodiments, the percentage GC content of the stem of the stem loop structure is greater than the percentage GC content of the loop of the stem loop structure. In some embodiments, the percentage GC content of the loop of the stem loop structure is about 28% to about 50%. In some embodiments, the percentage GC content of the loop of the stem loop structure is less than 50%. In some embodiments, the loop of the stem loop structure comprises about 1 to about 10 nucleotides. In some embodiments, the loop of the stem loop structure comprises about 3 to about 7 nucleotides. In some embodiments, the loop of the stem loop structure comprises 1, 2, 3, 4, 5, 6, 7, 8, 9, or 10 nucleotides. In some embodiments, the loop of the stem loop structure comprises the nucleotide sequence UCU or CUG. In some embodiments, the loop of the stem loop structure comprises the nucleotide sequence UCAGAUC. In some embodiments, the loop of the stem loop structure comprises at least 3, at least 4, at least 5, or at least 6 contiguous nucleotides from the sequence UCAGAUC.

ΔG can be calculated using methods available in the art to calculated folding energy of RNA secondary structure. ΔG can be determined experimentally using methods available in the art to measuring folding energy of RNA secondary structure.

In some embodiments, the first nucleotide of the secondary structure or stem is located at position +4, +5, +6, +7, +8, +9, +10, +11, +12, +13, +14, +15, +16, +17, +18, +19, +20, +21, +22, +23, +24, +25, +26, +27, +28, +29, +30, +31, +32, +33, +34, +35, +36, +37, or +38 relative to the first nucleotide of the start codon. In some embodiments, the first paired nucleotide of the secondary structure or stem is at about position +9 to about position +26 relative to the first nucleotide of the start codon. In some embodiments, the first paired nucleotide of the secondary structure or stem is at about position +13 to about position +20 relative to the first nucleotide of the start codon. In some embodiments, the first paired nucleotide of the secondary structure or stem is at about position +13 to about position +17 relative to the first nucleotide of the start codon. In some embodiments, the first paired nucleotide of the secondary structure or stem is at about position +15 relative to the first nucleotide of the start codon.

In some embodiments, the polypeptide comprises a therapeutic protein.

A DNA molecule encoding the described mRNA can further comprises promoter sequence operably linked to the sequence encoding the mRNA. The promoter can be, but is not limited to, a plant promoter, a plant virus promoter, a promoter from a non-viral plant pathogen, a mammalian cell promoter, or a mammalian virus promoter.

The mRNA or DNA molecule encoding the mRNA can be provided on a vector. The vector can be, but is not limited to, a viral vector, a transposon, a plasmid, or a CRISPR system. The vector can be any vector known in the art for expressing a nucleic acid sequence in a cell (e.g., a plant cell or a mammalian cell).

### III. POLYPEPTIDE/HETEROLOGOUS ORF

The heterologous (ORF) encoding the polypeptide operably linked to the described uORF can encode any polypeptide. In some embodiments, the polypeptide is a plant polypeptide. In some embodiments, the polypeptide is a mammalian polypeptide. In some embodiments, the polypeptide is an engineered or recombinant polypeptide.

In some embodiments, the heterologous gene comprises a gene whose expression increases resistance to plant disease, an infection, or a stress condition. The infection can be, but is not limited to, a viral infection, a bacterial infection, a fungal infection, an oomycete infection, a phytoplasma infection, or a nematode infection. The stress condition can be a biotic or an abiotic stress condition. The biotic stress condition can be, but is not limited to, infection or insect stress. The abiotic stress condition can be, but is not limited to, drought stress, heat stress, temperature stress (cold or heat), wind stress, pH stress, high salt stress, or nutrient deficiency.

In some embodiments, the heterologous gene comprises: MLO (e.g., TaMLO-B1), EDR1 (e.g., TaEDR1, OsEDR1), Pi21, OsSWEET11, OsSWEET13, OsSWEET14, eIF4E, DMR6 (SIDMR6-1), Sr35, Sr50, Sr33, Pik1 and Pik2, RGA5 and RGA4, RRS1 and RPS4, RBS1, CsLOB1, PBS1, Xa27, MAPK3K StVIK1, or COI1, including orthologs thereof in other plants (e.g., crop plants).

In some embodiments, the heterologous gene comprises a defense signaling and/or pathogenesis-related gene. Defense signaling and/or pathogenesis-related genes include, but are not limited to, IPA1, OsHEN1, SNC1, and NPR1, including orthologs thereof in other plants (e.g., crop plants).

In some embodiments, the heterologous gene comprises a Lesion mimic mutant (LMM) gene, a wild-type (non-mutant) allele of a LMM gene, or a suppressor of a LMM gene. LMM genes, wild-type alleles of LMM genes, and suppressors of LMM genes include, but are not limited to: cytidine diphosphate diacylglycerol (CDP-DAG) synthase encoding gene and RBL1. LMM genes in rice include, but are not limited to, BBS1, HPL3, CSLF6, LIL1, RLIN1, SPL3 (OsEDR1 ACDR1), LMS, OsLOL1, MLO, OsSL, LRD6-6, SPL5, SPL7, SPL11, SPL18, SPL28, ACLA2, ABC1, SPL33, OsSPL35, SPL40, WSP1, XB15, OsSSI2, GF14e, NOE1, EBR1, OsCUL3a, NLS1, or OsRLR1. LMM genes in Arabidopsis thaliana include, but are not limited to, SNC1, SSI4, SLH1, CHS3-2D, CHS3-1, CHS2, UNI-1D, BAK1, BKK1, BIR1, SNC4-1D, CERK1-4, SNC2-1D, RIN4, CPR1, SRFR1, CPN1/BON1, MKP1, LSD1, ACD11, CPR5, MEKK1, MPK4, MKK1, MKK2, ACD6, BDA1-17, DND1, DND2/HLM1, CPR22, NPR3 NPR4, SR1 CAMTA3, PUB13, CPR6-1, SSI2, SYP121, SYP122, CAD1, or NSL1. The heterologous gene can also be an ortholog of any of the above listed rice or Arabidopsis thaliana genes. In some embodiments, the ortholog is an ortholog in a commercial or crop plant.

In some embodiments, the polypeptide comprises a transcription factor, a reporter polypeptide, a polypeptide that confers resistance to drugs or agrichemicals, or a polypeptide involved in the growth or development of plants.

Any of the above disclosed heterologous genes can also be targets for in vivo genetic modification (see below).

### IV. METHODS

Described are methods for generating a cell comprising an inducibly-expressed polypeptide, the methods comprising introducing into the cell, any of the described expression constructs, mRNAs, DNAs, or vectors. In some embodiments, expression of the polypeptide is induced by a stress response in the cell. In some embodiments, expression of the polypeptide is induced by an immune response in the cell.

In some embodiments, expression of the polypeptide is induced by a stress-induced helicase in the cell. The stress-induced helicase can be an endogenous helicase expressed by the cell or a heterologous helicase. The helicase can be, but is not limited to, a RH37 helicase or an ortholog thereof, a RH11 helicase or an ortholog thereof, a RH52 helicase or an ortholog thereof, or a Ded1p helicase or an ortholog thereof.

In some embodiments, expression of the polypeptide is induced by an immune-induced helicase in the cell. The immune-induced helicase can be an endogenous helicase expressed by the cell or a heterologous helicase. The helicase can be, but is not limited to, a DDX3X helicase or an ortholog thereof, or a DEAD-box family helicase or an ortholog thereof.

In some embodiments, the methods further comprise expressing a heterologous helicase in the cell or contacting the cell with an ASO that specifically hybridizes to a sequence in the secondary structure or stem loop structure. The helicase can be, but is not limited to, stress-induced helicase, or an immune response-induced helicase. The ASO specifically hybridizes to all or a portion of the first stem sequence, all or a portion of the second stem sequence, or a combination thereof. The ASO hybridizes to the secondary structure or stem loop sequence with sufficient affinity to disrupt formation of the secondary structure or stem loop structure.

Various methods for introducing an expression construct, vector, or CRISPR/Cas system into a plant or plant cell are well known to those skilled in the art. Nucleic acids may be introduced (transformed) into plants and plants cells using a number of methods known in the art, including, but not limited to, electroporation (U.S. Pat. No. 5,384,253, incorporated herein by reference), microprojectile bombardment or biolistic approaches (U.S. Pat. Nos. 5,550,318, 5,538,877, 5,538,880, 5,610,042, and PCT Application WO 94/09699; each incorporated herein by reference), various DNA-based vectors such as Agrobacterium tumefaciens vectors (U.S. Pat. Nos. 5,591,616 and 5,563,055; each incorporated herein by reference), and silicon carbide fiber transformation. In some embodiments, embryogenic callus, leaf whorls, whole plants, plant tissue culture cells, immature embryo, or friable tissue are transformed using one of the above methods. Additional methods include, but are not limited to, protoplast transformation of naked DNA by calcium, polyethylene glycol (PEG), or electroporation. Once a plant cell has been successfully transformed, it may be cultivated to regenerate a transgenic plant (regenerant). The plants may be reproduced, either sexually or asexually, using methods well known in the art, to produce successive generations of transformed plants.

Similarly, various methods for introducing an expression construct, vector, or CRISPR/Cas system into a mammal or mammalian cell are well known to those skilled in the art. Nucleic acids may be introduced (transformed) into mammals or mammalian cells using a number of methods known in the art.

Also described are methods for generating a cell in which a polypeptide is inducibly expressed or altering expression of an endogenous gene in a cell. The methods comprise modifying an endogenous gene encoding the polypeptide to produce a modified gene, wherein the modified gene encodes an mRNA comprising (a) a heterologous upstream open reading frame (uORF) comprising an upstream start codon and a sequence that forms a stem loop structure operably linked to the upstream start codon, wherein the stem loop structure is about 1 to about 35 nucleotides downstream of the upstream start codon, and wherein the stem loop structure comprises a stem having about 12 to about 20 base pairs; and (b) an open reading frame (ORF) encoding the polypeptide, wherein the heterologous uORF is 5′ of the ORF and is operably linked to the ORF. In some embodiments, the cell is a plant cell. In some embodiments, the cell is a mammalian cell.

In some embodiments, the methods comprise modifying an endogenous gene encoding the polypeptide to produce a modified gene, wherein the modified gene encodes an mRNA comprising (a) a heterologous upstream open reading frame (uORF) comprising an upstream start codon and a sequence that forms a secondary structure operably linked to the upstream start codon, wherein the secondary structure begins about 1 to about 35 nucleotides downstream of the upstream start codon and has a folding energy of about −19.9 kcal mol⁻¹ to about −34.1 kcal mol⁻¹ when calculated for nucleotides +4 to +104 relative to the upstream start codon; and (b) an open reading frame (ORF) encoding the polypeptide, wherein the heterologous uORF is 5′ of the ORF and is operably linked to the ORF. In some embodiments, the cell is a plant cell. In some embodiments, the cell is a mammalian cell.

The uORF start codon may be any codon known in the art that can use used as a start codon. In some embodiments, the uORF start codon is AUG (uAUG). In some embodiments, the uORF start codon is a non-uAUG start codon. A uORF non-AUG start codon can be, but is not limited to, CUG, GUG, ACG, UUG, AUU, AUC, AAG, AUA, or ΔGG.

The uORF start codon may be linked to, or present in the context of, a strong Kozak sequence, an average Kozak sequence, a weak Kozak sequence, or no identifiable Kozak sequence. A strong Kozak sequence indicates the nucleotide at position +4 (one nucleotide downstream of the start codon) is a consensus Kozak sequence nucleotide (e.g., a G) and the nucleotide at position −3 (three nucleotides upstream of the start codon) is a consensus Kozak sequence nucleotide (e.g., an A). An average Kozak sequence indicates that either the nucleotide at position +4 is a consensus Kozak sequence nucleotide or the nucleotide at position −3 is a consensus Kozak sequence nucleotide (e.g., an A), but not both. A weak Kozak sequence indicates that neither the nucleotide at position +4 nor the nucleotide at position and −3 is a consensus Kozak sequence nucleotide. A Kozak sequence can be, but is not limited to, (A/G)cc[start codon]G, wherein the start codon corresponds to positions +1, +2, and +3 (see Table 1).

In some embodiments, the stem loop structure of the uORF comprises a first stem sequence, a loop sequence, and a second stem sequence. The first and second stem sequences can be about 12 to about 24 nucleotides in length. In some embodiments, the stem of the stem loop structure of the uORF comprises about 12 to about 20 base pairs and starts about 1 to about 35 nucleotides downstream of the uORF start codon (i.e., the first nucleotide of the stem is located at about position +4 to about +38), wherein the first nucleotide of the upstream start codon is +1. The about 12 to about 20 base pairs in the stem can be contiguous or discontiguous. If discontiguous, the stem can comprise 12 to 20 base pairs (24 to 40 paired nucleotides) and about 1 to about 10 unpaired nucleotides. In some embodiments, the stem of the stem loop structure of the uORF contains no unpaired or mismatched nucleotides. In some embodiments, the stem of the stem loop structure of the uORF contains at least one unpaired or mismatched nucleotide. In some embodiments, the stem of the stem loop structure of the uORF contains 12, 13, 14, 15, 16, 17, 18, 19, or 20 base pairs. In some embodiments, the stem of the stem loop structure of the uORF contains no more than 10, no more than 9, no more than 8, no more than 7, no more than 6, no more than 5, no more than 4, no more than 3, no more than 2, or no more than 1 unpaired or mismatched nucleotides. In some embodiments, the stem of the stem loop structure of the uORF comprises about 12 to about 20 base pairs and/or has a folding energy of (ΔG) about −19.9 kcal mol⁻¹ to about −34.1 kcal mol⁻¹. In some embodiments, the stem of the stem loop structure of the uORF comprises about 12 to about 20 base pairs and has a folding energy of −27±7 kcal mol-1. In some embodiments, the stem of the stem loop structure of the uORF comprises about 12 to about 20 base pairs and has a folding energy of −27±6 kcal mol⁻¹. In some embodiments, the stem of the stem loop structure of the uORF comprises about 12 to about 20 base pairs and has a folding energy of about −26.8±5 kcal mol⁻¹. In some embodiments, the stem of the stem loop structure of the uORF comprises about 12 to about 20 base pairs, wherein the percentage GC content of the stem of the stem loop structure is about 46% to about 61%. In some embodiments, the percentage GC content of the stem of the stem loop structure is at least 50%. In some embodiments, the percentage GC content of the stem of the stem loop structure is greater than the percentage GC content of the loop of the stem loop structure. In some embodiments, the percentage GC content of the loop of the stem loop structure is about 28% to about 50%. In some embodiments, the percentage GC content of the loop of the stem loop structure is less than 50%. In some embodiments, the loop of the stem loop structure comprises about 1 to about 10 nucleotides. In some embodiments, the loop of the stem loop structure comprises about 3 to about 7 nucleotides. In some embodiments, the loop of the stem loop structure comprises 1, 2, 3, 4, 5, 6, 7, 8, 9, or 10 nucleotides. In some embodiments, the loop of the stem loop structure comprises the nucleotide sequence UCU or CUG. In some embodiments, the loop of the stem loop structure comprises the nucleotide sequence UCAGAUC. In some embodiments, the loop of the stem loop structure comprises at least 3, at least 4, at least 5, or at least 6 contiguous nucleotides from the sequence UCAGAUC.

ΔG can be calculated using methods available in the art to calculated folding energy of RNA secondary structure. ΔG can be determined experimentally using methods available in the art to measuring folding energy of RNA secondary structure.

The stem of the stem loop structure of the uORF can comprise the nucleotide sequence of any of the stem structures disclosure herein, provided the stem contains 12 to 20 base pairs and/or has a folding energy of about −19.9 kcal mol⁻¹ to about −34.1 kcal mol⁻¹. The stem loop structure of the uORF can comprise the nucleotide sequence of any of the stem loop structures disclosure herein, provide the stem contains 12 to 20 base pairs and/or has a folding energy of about −19.9 kcal mol⁻¹ to about −34.1 kcal mol⁻¹. In some embodiments, the stem loop structure comprises GACGTCGGTTCCGACGTC (SEQ ID NO: 73).

In some embodiments, the first nucleotide of the secondary structure or stem is located at position +4, +5, +6, +7, +8, +9, +10, +11, +12, +13, +14, +15, +16, +17, +18, +19, +20, +21, +22, +23, +24, +25, +26, +27, +28, +29, +30, +31, +32, +33, +34, +35, +36, +37, or +38 relative to the first nucleotide of the upstream start codon. In some embodiments, the first paired nucleotide of the secondary structure or stem is at about position +9 to about position +26 relative to the first nucleotide of the upstream start codon. In some embodiments, the first paired nucleotide of the secondary structure or stem is at about position +13 to about position +20 relative to the first nucleotide of the upstream start codon. In some embodiments, the first paired nucleotide of the secondary structure or stem is at about position +13 to about position +17 relative to the first nucleotide of the upstream start codon. In some embodiments, the first paired nucleotide of the secondary structure or stem is at about position +15 relative to the first nucleotide of the upstream start codon. In some embodiments, the secondary structure or stem loop structure of the uORF is positioned such that a ribosome, or ribosome subunit, scanning the 5′ UTR of an mRNA containing the uORF pauses with the P site of the ribosome at or in proximity of the uORF start codon, thereby resulting in increased initiation of translation of the uORF.

In some embodiments, expression of the polypeptide is induced by a stress response in the cell. In some embodiments, translation of the ORF is increased by a stress response in the cell.

In some embodiments, expression of the polypeptide is induced by an immune response in the cell. In some embodiments, translation of the ORF is increased by an immune response in the cell.

In some embodiments, expression of the polypeptide is induced by a stress-induced helicase in the cell. In some embodiments, translation of the ORF is increased by a stress-induced helicase in the cell. The stress-induced helicase can be an endogenous helicase expressed by the cell or a heterologous helicase. The helicase can be, but is not limited to, a RH37 helicase or an ortholog thereof, a RH11 helicase or an ortholog thereof, a RH52 helicase or an ortholog thereof, or a Ded1p helicase or an ortholog thereof. In some embodiments, the method further comprises expressing a heterologous stress-induced helicase in the cell.

In some embodiments, expression of the polypeptide is induced by an immune-induced helicase in the cell. In some embodiments, translation of the ORF is increased by an immune-induced helicase in the cell. The immune-induced helicase can be an endogenous helicase expressed by the cell or a heterologous helicase. The helicase can be, but is not limited to, a DDX3X helicase or an ortholog thereof, or a DEAD-box family helicase or an ortholog thereof. In some embodiments, the method further comprises expressing a heterologous immune-induced helicase in the cell.

In some embodiments, expression of the polypeptide is induced by an antisense oligonucleotide (ASO) that specifically hybridizes to a sequence in the secondary structure or stem loop structure. In some embodiments, translation of the heterologous ORF is increased by an ASO that specifically hybridizes to a sequence in the secondary structure. In some embodiments, translation of the heterologous ORF is increased by an ASO that specifically hybridizes to a sequence in the stem loop structure. The ASO can be delivered to the cell to increase expression of the polypeptide (i.e., increase translation of the heterologous ORF). The ASO can specifically hybridize to all or a portion of the first stem sequence, all or a portion of the second stem sequence, or a combination thereof. The ASO hybridizes to the stem loop sequence with sufficient affinity to disrupt formation of the stem loop structure. In some embodiments, the method further comprises contacting the cell with a ASO that specifically hybridizes to a sequence in the stem loop structure.

The gene can be, but is not limited to, any of the polypeptide/heterologous ORFs described above.

Modifying an endogenous gene in a cell may be done using any method available in the art for modifying an endogenous gene. Such methods include, but are not limited to CRISPR-mediated methods (e.g., CRISPR/Cas). For example, an endogenous gene can be modified by recombination with or insertion of exogenous DNA molecule. Repair in response to double-strand breaks (DSBs) occurs principally through two conserved DNA repair pathways: homologous recombination (HR) and non-homologous end joining (NHEJ). Homology directed repair (HDR) or homologous recombination (HR) include a form of nucleic acid repair that can require nucleotide sequence homology, uses a “donor” molecule as a template for repair of a “target” molecule, and leads to transfer of genetic information from the donor to target. Non-homologous end joining (NHEJ) includes the repair of double-strand breaks in a nucleic acid by direct ligation of the break ends to one another or to an exogenous sequence without the need for a homologous template. Ligation of non-contiguous sequences by NHEJ can often result in deletions, insertions, or translocations near the site of the double-strand break. For example, NHEJ can also result in the targeted integration of an exogenous donor nucleic acid through direct ligation of the break ends with the ends of the exogenous donor nucleic acid (i.e., NHEJ-based capture). Such NHEJ-mediated targeted integration can be preferred for insertion of an exogenous donor nucleic acid when homology directed repair (HDR) pathways are not readily usable (e.g., in non-dividing cells, primary cells, and cells which perform homology-based DNA repair poorly).

Also described are methods for increasing translation of a gene having an upstream open reading frame comprising an upstream start codon and a sequence that forms a secondary structure or stem loop structure operably linked to the upstream start codon, wherein the secondary structure or stem loop structure is about 1 to about 35 nucleotides downstream of the upstream start codon, the methods comprising contacting a cell containing the gene with an antisense oligonucleotide that specifically hybridizes to a sequence in the secondary structure or stem loop structure. The ASO can specifically hybridize to all or a portion of a first stem sequence of the stem loop structure, all or a portion of a second stem sequence of the stem loop structure, or a combination thereof. The ASO hybridizes to the secondary structure or stem loop sequence with sufficient affinity to disrupt formation of the secondary structure or stem loop structure. The gene can be an endogenous gene or a heterologous gene.

Also described are methods for increasing translation of an ORF in a cell, the method comprising: modifying a nucleic acid encoding the ORF to contain a stem loop structure, wherein the stem loop structure is operably linked to the start codon of the ORF, is about 1 to about 35 nucleotides downstream of the start codon, and comprises a stem having about 12 to about 20 base pairs. In some embodiments, the method comprises substituting one or more codons downstream of the ORF thereby forming the stem loop structure. In some embodiments, substituting the one or more codons does not change the encoded amino acid sequence or results in one or more conservative amino acids changes to the coding sequence.

Also described are methods for increasing translation of an ORF in a cell, the method comprising: modifying a nucleic acid encoding the ORF to contain a secondary structure, wherein the secondary structure is operably linked to the start codon of the ORF, begins about 1 to about 35 nucleotides downstream of the start codon, and has a folding energy of about −19.9 kcal mol⁻¹ to about −34.1 kcal mol⁻¹ when calculated for nucleotides +4 to +104 relative to the start codon. In some embodiments, the method comprises substituting one or more codons downstream of the ORF thereby forming the secondary structure. In some embodiments, substituting the one or more codons does not change the encoded amino acid sequence or results in one or more conservative amino acids changes to the coding sequence.

In some embodiments, methods of producing genetically modified plants having a protein whose expression is increased in response to stress are described, the methods comprising, modifying an endogenous gene to contain an uORF, wherein the uORF comprises an upstream start codon, and a sequence that forms a stem loop structure operably linked to the upstream start codon, wherein the endogenous gene encodes the protein. The stem loop structure can be about 1 to about 35 nucleotides downstream of the upstream start codon. The stem loop structure can comprise a stem having about 12 to about 20 base pairs and/or a folding energy (ΔG) of about −19.9 kcal mol⁻¹ to about −34.1 kcal mol⁻¹.

In some embodiments, methods of producing genetically modified plants having a protein whose expression is increased in response to stress are described, the methods comprising, modifying an endogenous gene to contain an uORF, wherein the uORF comprises an upstream start codon, and a sequence that forms a secondary structure operably linked to the upstream start codon, wherein the endogenous gene encodes the protein. The secondary structure begins about 1 to about 35 nucleotides downstream of the upstream start codon and has a folding energy of about −19.9 kcal mol⁻¹ to about −34.1 kcal mol⁻¹ when calculated for nucleotides +4 to +104 relative to the upstream start codon.

In some embodiments, methods of producing genetically modified plants having a protein whose expression is increased in response to stress are described, the methods comprising, (a) forming a DNA molecule the encoding an mRNA, wherein the mRNA comprises (i) an upstream uORF comprising an upstream start codon, and a sequence that forms a stem loop structure operably linked to the upstream start codon; and (ii) a heterologous ORF encoding a protein, wherein the uORF is 5′ of the heterologous ORF and is operably linked to the heterologous ORF; and (b) introducing the DNA molecule into a plant or plant cell or plant tissue. The stem loop structure can be about 1 to about 35 nucleotides downstream of the upstream start codon. The stem loop structure can comprise a stem having about 12 to about 20 base pairs and/or a folding energy (ΔG) of about −19.9 kcal mol⁻¹ to about −34.1 kcal mol-.

In some embodiments, methods of producing genetically modified plants having a protein whose expression is increased in response to stress are described, the methods comprising, (a) forming a DNA molecule the encoding an mRNA, wherein the mRNA comprises (i) an upstream uORF comprising an upstream start codon, and a sequence that forms a secondary structure operably linked to the upstream start codon; and (ii) a heterologous ORF encoding a protein, wherein the uORF is 5′ of the heterologous ORF and is operably linked to the heterologous ORF; and (b) introducing the DNA molecule into a plant or plant cell or plant tissue. In some embodiments, the secondary structure can begin about 1 to about 35 nucleotides downstream of the upstream start codon and has a folding energy (ΔG) of about −19.9 kcal mol⁻¹ to about −34.1 kcal mol⁻¹.

In some embodiments, translation initiation from the upstream start codon is higher in the absence of stress relative to translation initiation from the upstream start codon in the presence of stress, resulting in decreased translation of the protein in the absence of stress relative to the translation of the protein in the presence of stress. In some embodiments, destabilization of the secondary structure or stem loop structure in the presence of stress—e.g., due to expression of a stress-induced helicase-decreases translation initiation from the upstream start codon, thereby increasing translation of the protein. In some embodiments, the protein comprises a defense (i.e., immune) or defense-related protein. In some embodiments, the stress comprises a pathogen challenge.

In some embodiments, methods of producing genetically modified plants having a protein whose expression decreased in response to stress are described, the methods comprising, genetically modifying an endogenous gene in the plant to contain a sequence that forms a stem loop structure operably linked to the start codon, wherein the endogenous gene encodes the protein. The stem loop structure can be about 1 to about 35 nucleotides downstream of the upstream start codon. The stem loop structure can comprise a stem having about 12 to about 20 base pairs and/or a folding energy (ΔG) of about −19.9 kcal mol⁻¹ to about −34.1 kcal mol⁻¹.

In some embodiments, methods of producing genetically modified plants having a protein whose expression decreased in response to stress are described, the methods comprising, genetically modifying an endogenous gene in the plant to contain a sequence that forms a secondary structure operably linked to the start codon, wherein the endogenous gene encodes the protein. In some embodiments, the secondary structure begins about 1 to about 35 nucleotides downstream of the upstream start codon and has a folding energy (ΔG) of about −19.9 kcal mol¹ to about −34.1 kcal mol-1.

In some embodiments, methods of producing genetically modified plants having a protein whose expression decreased in response to stress are described, the methods comprising, (a) forming a DNA molecule the encoding an mRNA, wherein the mRNA comprises a sequence that forms a stem loop structure operably linked to a start codon for an ORF encoding the protein; and (b) introducing the DNA molecule into a plant or plant cell or plant tissue. The stem loop structure can be about 1 to about 35 nucleotides downstream of the upstream start codon. The stem loop structure can comprise a stem having about 12 to about 20 base pairs and/or a folding energy (ΔG) of about −19.9 kcal mol⁻¹ to about −34.1 kcal mol-1.

In some embodiments, methods of producing genetically modified plants having a protein whose expression decreased in response to stress are described, the methods comprising, (a) forming a DNA molecule the encoding an mRNA, wherein the mRNA comprises a sequence that forms a secondary structure operably linked to a start codon for an ORF encoding the protein; and (b) introducing the DNA molecule into a plant or plant cell or plant tissue. In some embodiments, the secondary structure begins about 1 to about 35 nucleotides downstream of the upstream start codon and has a folding energy (ΔG) of about −19.9 kcal mol⁻¹ to about −34.1 kcal mol-1.

In some embodiments, translation initiation from the start codon is higher in the absence of stress relative to translation initiation from the start codon in the presence of stress, resulting in increased translation of the protein in the absence of stress relative to the translation of the polypeptide in the presence of stress. In some embodiments, destabilization of the secondary structure or the stem loop structure in the presence of stress, e.g., due to expression of a stress-induced helicase, decreases translation initiation from the start codon, thereby decreasing translation of the protein. In some embodiments, the polypeptide comprises a plant growth or plant growth-related protein. The ORF encoding the protein may contain start codon that is linked to, or present in the context of, a strong Kozak sequence, an average Kozak sequence, a weak Kozak sequence, or no identifiable Kozak sequence. In some embodiments, the stress comprises a pathogen challenge.

The methods described for producing genetically modified plants can also be used to produce genetically modified plant cells, genetically modified plant tissue, genetically modified mammalian cells, genetically modified mammalian tissue, or genetically modified mammals.

### V. MODIFIED CELLS

The described expression constructs, DNA molecules, vectors, and cells can be used to generate a genetically modified cell, plant, or mammal. The genetically modified cell can be a plant cell or a mammalian cell. The plant cell can be in a plant. The genetically modified plant cell can be used to generate a genetically modified plant. The mammalian cell can be in a mammal. The genetically modified mammalian cell can be used to generate a genetically modified mammal.

In some embodiments, modified cells are provided, wherein the modified cells comprise any of the described expression constructs, mRNAs, DNA, vectors, or modified endogenous genes. The modified cell can be a plant cell or a mammalian cell. The modified cell can be in a plant or a mammal.

In some embodiments, plant propagation materials are provided, wherein the plant propagation materials comprising one or more plant cells comprising any of the described expression constructs, mRNAs, DNA, vectors, or modified endogenous genes.

In some embodiments, a modified or transgenic plant is provided, wherein the modified or transgenic plant comprises any of the described expression constructs, mRNAs, DNA, vectors, or modified endogenous genes, one or more cells comprising any of the described expression constructs, mRNAs, DNA, vectors, or modified endogenous genes. In some embodiments, a modified or transgenic plant is provided, wherein the modified or transgenic plant comprises a plant generated from one or more cells or plant propagation materials, wherein the one or more cells or plant propagation materials have been modified to comprise any of the described expression constructs, mRNAs, DNA, vectors, or modified endogenous genes.

Described below, in Tables 2-5, are exemplary downstream sequences (corresponding to positions +4 to +X of a uORF or mORF) in mRNAs that form suitable secondary structures. In some embodiments, the mRNAs described herein can have a uORF comprising any of SEQ ID NOs:66-72 and 74-93. In some embodiments, any of the DNAs described herein can encode an mRNA having a uORF comprising any of SEQ ID NOs: 66-72 and 74-93. In some embodiments, the mRNAs described herein can have a mORF comprising any of SEQ ID NOs:66-72 and 74-93. In some embodiments, any of the DNAs described herein can encode an mRNA having a mORF comprising any of SEQ ID NOs: 66-72 and 74-93.

TABLE 2
Exemplary downstream sequences (positions +4 to +X) having sequences that form
secondary structures.

| SEQ ID | |
|---|---|
| NO: | sequence |
| 74 | GAGAUAGAAGCAAGCAGACAACAAACCACCGUACCGGUUUCAGUCGGCGGUGGGAAUUUUCCGGUU |
| | GGUGGGUUAAGUCCGUUGAGUGAAGCUAUAUGGAG |
| 75 | AAAUGGGAGAAAUGGAGAUCGAAGAAAUCGAAGCUGUUCUUGAGAAAAUCUGGGAUCUACAUGACA |
| | AGCUUAGCGAUGAGAUUCACUUGAUUUCGAAGUCU |
| 76 | AGUUCUUCAGAGAGUGUGGAAAACGAGUGCAUGUGUUGGGCUGCAAGAGAUCCAUCUGGUCUUCUU |
| | UCUCCUCAUACUAUCACUCGCAGGUCUGUUACAAC |
| 77 | UGUUAGAGAUAUCCGGAGAUCUCAACCGUUGGAUUCUUUCUCCUUCAAAUCAAAUUAUAAAUCCCA |
| | UCAAACAGAGAGAGAGAGAGAAGAGGAGGAGAAUU |
| 78 | GAGAGUAUCUGGCGAAUCGCGACGGGACAAGAUCCGAGCCGUGAAGAUUACGAAGGGAUCGAGUUC |
| | UGGUCAAACCCUGAGCGUUCUGGUUGGCUCACAAA |
| 79 | CUAAAUUGAGUUUCGUCGUCUUCUGCUUCUGCUUAAGCUUCUUCAUCAUUUAGCUCCAGAGGCGGA |
| | GCUUUCUUCUCUCUCUGUGGACGACCAUGGCGGCG |
| 80 | CAAAGCUCAAUGACAAUGGAACUACGACCAUCAGGUGAUUCCGGUUCAUCUGACGUCGAUGCUGAG |
| | AUCAGCGAUGGCUUUUCACCGCUCGAUACUUCUCA |
| 81 | GCGUUGCUGAAGUCUUUCAUCGAUGUUGGCUCAGACUCGCACUUCCCUAUCCAGAAUCUCCCUUAU |
| | GGUGUCUUCAAACCGGAAUCGAACUCAACUCCUCG |
| 82 | CAGCUUCAGGAAAUCCAAGAUAACAUAAGGAGUAGACGCAAUAAGAUAUUUCUCCUCAUGGAGGAA |
| | GUGAGGAGGUUACGUGUGCAGCAACGUAUCAAGAG |
| 83 | UAUCCUUCUCUCGACGAUGAUUUCGUCUCUGAUUUGUUUUGCUUCGAUCAAAGCAAUGGAGCAGAA |
| | CUUGAUGAUUACACACAGUUUGGUGUAAAUUUGCA |
| 84 | AAAUUGGAUUACGAUGGCUGGAUAUCCUACUAAUGGAUCAGUCUACGUUUCAAAUCUCCCCUUAGG |
| | AACUGACGAGAAUAUGUUGGCUGACUAUUUUGGGA |
| 85 | GAGCGUCUAACAUCUCCUCCUCGUUUGAUGAUUGUCUCUGAUCUUGAUCAUACUAUGGUUGAUCAU |
| | CAUGAUCCUGAGAAUCUAUCUCUGCUGAGAUUCAA |
| 86 | UGAGAAGAAAAUGUCAGGAUCUGAGACGGGUUUAAUGGCGGCGACCAGAGAAUCAAUGCAAUUUAC |
| | AAUGGCUCUCCACCAGCAGCAGCAACACAGUCAAG |
| 87 | UCGACGGUGUACGUGCUAGAGCCGCCGACAAAGGGAAAGGUCAUUGUAAAUACUACUCAUGGUCCA |
| | AUCGACGUCGAGCUUUGGCCUAAGGAAGCGCCCAA |
| 88 | UCGGCGGAGAGUUGUUUCGGAAGCUCGGGUGAUCAAAGCAGCAGCAAAGGAGUGGCUACUCAUGGU |
| | GGUAGCUAUGUUCAGUAUAAUGUCUAUGGCAAUCU |
| 89 | GAAAGCUUGGACACUAAUUUUCCUGUGCGCCAUAGAAAGGUCUCGUUUGAAAGUAAGGGAAACAAG |
| | ACAGAGAUUGUGAUCUGCAGCUAUGAAGAUCAUAU |
| 90 | GGUUUCUUCUCUUUUCUUGGAAGAGUUCUCUUCGCUUCUUUAUUCAUCCUCUCCGCUUGGCAAAUG |
| | UUCAAUGACUUUGGAACUGACGGUGGUCCAGCAGC |
| 91 | AAUACUGGUGGUCGGCUCAUCGCUGGUUCUCACAAUAGGAAUGAGUUCGUGUUGAUUAAUGCAGAC |
| | GAGAGUGCCAGAAUUAGAUCAGUGGAAGAACUAAG |
| 92 | GCUUCUGUUAUCUCUUCCUCUCCUUUUCUAUGCAAAUCAUCCUCCAAGAGUGAUUUGGGGAUUUCU |
| | UCGUUUCCUAAAUCUUCUCAGAUUUCGAUUCAUCG |
| 93 | AGUCUCUUCAACACUGAAAACACAUGGGCCUUUGUCUUUGGCUUGCUCGGCAACCUUAUCUCCUUU |
| | GCCGUGUUCCUAUCUCCUGUGCCAACGUUCUAUAG |

TABLE 3
Base paring for exemplary downstream sequences

| SEQ ID | Base paring |
|---|---|
| NO: | · = unpaired nucleotide \| ( and ) = paired nucleotides |
| 74 | ..........................((((..(((((((.(((((((..........((....))..........))))))).))))))).))))... |
| 75 | ...............((((....((((((..((...(((....)))...)).((((((.(((..(.......)..)))))))))....))))))...)))) |
| 76 | .(((.(((.........))).)))(((((...((((.(((..(.(((((..((....))..))))).).))).))))...)))))................ |
| 77 | .(((.(((((........))))))))(.((...(((((((((((((...........................)).)).)))))))))...)).)...... |
| 78 | .(((...))).(((...)))(((.((((..(((((.....((((.....))))...)))))..))))..))).......(((((..........))))).... |
| 79 | ..............(((((((.(((((....))...................((((((......))))))...............))).)))...))))... |
| 80 | ........................((((..((((((((.......)))))))).))))..(((((((...((....)).)))))..))............. |
| 81 | (((...((((.(((...((....))..)))))))...))).............(((....((....((....))....))).....(((............)))... |
| 82 | ...(((..((....))))).((((...............((((...)))).((((((((.......))))))))))))...((((......))))...... |
| 83 | ............(((((.....)))))((......((((((((((............)))))))))).....)).(((((........)))))........ |
| 84 | .......(((((((..(((((...((((.......)))))))))..)))....)))).(((..(((..((((((........))))).)..)))....))). |
| 85 | (((.((...))..)))........((((.((((.((...((((..(((((..(...)..))))).....))))..)).)))).(((((....))))))))) |
| 86 | .............((((...))))(((.((((.....((((.(....(((..(((.((.......)).)))..)))).)).))......))).).)))... |
| 87 | .....(((((...(.((((.(((((((((.....((..((((((.(((.....)))..)))).))..))...)))).))...)))))).).).)))))... |
| 88 | .......(((..((((..(((.((((.(.(......).).).)))...(((.((((((((......))))))))..))).........)))..)))).))) |
| 89 | .....(((((.(((..........)))..))).))....((((.((((...........)))).)))).........(((((.((....)).))))).... |
| 90 | ...................(((((....)))))(((.((....(((.((...((....)).....((((..........))))....)))))...)).))) |
| 91 | ....(((((.....((((.(((((((((((.......)))))...................))).))))))))))))........................ |
| 92 | ..................................((((....((..((((.(((((((((((......)))))).)))))))))..))....))))..... |
| 93 | ..................(((...(((..(.........((...((.((((((...........)))))).)).))........)..)))..)))...... |

TABLE 4
Properties of exemplary downstream sequences.

| | | secondary | base pairs | | |
|---|---|---|---|---|---|
| | folding | structure | in | % GC | % GC |
| SEQ ID | energy | starting | secondary | content | content |
| NO: | (kcal/mol) | position | structure | in stem | in loop |
| 74 | −18.67 | +33 | 20 | 57.5% | 50.0% |
| 75 | −27.52 | +19 | 20 | 40.0% | 28.6% |
| 76 | −34.15 | +28 | 20 | 57.5% | 25.0% |
| 77 | −19.89 | +30 | 16 | 43.8% | 22.2% |
| 78 | −27.02 | +24 | 16 | 62.5% | 20.0% |
| 79 | −34.23 | +17 | 16 | 68.8% | 66.7% |
| 80 | −22.23 | +28 | 12 | 54.2% | 57.1% |
| 81 | −26.99 | +4 | 12 | 70.8% | 50.0% |
| 82 | −32.52 | +24 | 12 | 45.8% | 57.1% |
| 83 | −35.52 | +31 | 12 | 50.0% | 33.3% |
| 84 | −33.09 | +10 | 16 | 53.1% | 14.3% |
| 85 | −33.91 | +28 | 20 | 40.0% | 0.0% |
| 86 | −25.07 | +28 | 20 | 67.5% | 14.3% |
| 87 | −26.56 | +9 | 30 | 65.0% | 20.0% |
| 88 | −27.04 | +11 | 21 | 47.6% | 50.0% |
| 89 | −19.82 | +9 | 8 | 62.5% | 20.0% |
| 90 | −19.64 | +37 | 14 | 60.7% | 30.0% |
| 91 | −19.72 | +8 | 20 | 60.0% | 28.6% |
| 92 | −31.33 | +38 | 21 | 38.1% | 33.3% |
| 93 | −21.46 | +22 | 17 | 67.6% | 45.5% |

TABLE 5
uORF having stem loop structures of difference strengths (ds region underlined).

| SEQ ID | | stem loop |
|---|---|---|
| NO. | SEQUENCE | folding energy |
| 66 | CTCCTCTTCTTCTCTCTCTCATAAAACAAAAGATCCTCTCTGCTTCTCCTTTC | −9.8 kcal/mol |
| | CCTTTTTCAAAATCTCTCTCTCTCTCTCTCTTTTTAGATCCAGTTTTAGGGTT | |
| | TTCTTCTCTGTGAGCGAAG | |
| 67 | CTCCTCTTCTTCTCTCTCTCATAAAACAAAAGATCCTCTCTGCTTCTCCTTTC | −16.9 kcal/mol |
| | GGTTCCGAAAAATCTCTCTCTCTCTCTCTCTTTTTAGATCCAGTTTTAGGGT | |
| | TTTCTTCTCTGTGAGCGAAG | |
| 68 | CTCCTCTTCTTCTCTCTCTCATAAAACAAAAGATCCTCTCTGCTTCTGACGT | −23.6 kcal/mol |
| | CGGTTCCGACGTCTCTCTCTCTCTCTCTCTCTTTTTAGATCCAGTTTTAGGGT | |
| | TTTCTTCTCTGTGAGCGAAG | |
| 69 | CTCCTCTTCTTCTCTCTCTCATAAAACAAAAGATCCTCTCTGCGTGGGACGT | −33.2 kcal/mol |
| | CGGTTCCGACGTCCCACTCTCTCTCTCTCTCTTTTTAGATCCAGTTTTAGGG | |
| | TTTTCTTCTCTGTGAGCGAAG | |
| 70 | CTCCTCTTCTTCTCTCTCTCATAAAACAAAAGATCCTCTGAGCGTGGGACGT | −41.8 kcal/mol |
| | CGGTTCCGACGTCCCACGCTCTCTCTCTCTCTTTTTAGATCCAGTTTTAGGG | |
| | TTTTCTTCTCTGTGAGCGAAG | |
| 71 | CTCCTCTTCTTCTCTCTCTCATAAAACAAAAGATCCTCCGAGCGTGGGACGT | −52.1 kcal/mol |
| | CGGTTCCGACGTCCCACGCTCGGAGTCTCTCTTTTTAGATCCAGTTTTAGGG | |
| | TTTTCTTCTCTGTGAGCGAAG | |
| 72 | CTCCTCTTCTTCTCTCTCTCATAAAACAAAAGACCCTCCGAGCGTGGGACG | −62.4 kcal/mol |
| | TCGGTTCCGACGTCCCACGCTCGGAGGGTCTCTTTTTAGATCCAGTTTTAGG | |
| | GTTTTCTTCTCTGTGAGCGAAG | |

The following examples are provided to illustrate certain particular features and/or embodiments. These examples should not be construed to limit the disclosure to the particular features or embodiments described.

### EXAMPLES

Translation of eukaryotic genes is regulated by multiple features in the mRNAs. Among them, upstream start codons (uAUGs) and associated open reading frames (uORFs) are widely present in the 5′ leader sequences (~64% in human, ~60% in mouse, ~55% in Drosophila, ~54% in Arabidopsis and −31% in rice). Most eukaryotic mRNAs are translated in a cap-dependent manner, in which the 43S preinitiation complex scans the mRNA from the 5′ cap and initiates translation at a start codon by recruiting the 60S ribosomal subunit. The presence of uAUGs provides potential alternative sites for the preinitiation complex to start translation before it reaches the main AUG (mAUG); and if translation initiates from uAUGs, it would typically inhibit translation from the downstream mAUGs. This inhibitory role of uAUGs is critical for controlling the production of certain proteins under normal conditions, such as those related to stress response or cell death. For example, constitutive translation of the key plant immune transcription factor TL1-binding factor (TBF1) without the two uAUGs/uORFs in its 5′ leader sequence causes lethality. Interestingly, the majority of uORFs do not have conserved sequences despite undergoing positive Darwinian selection, suggesting that they inhibit main ORF (mORF) translation mostly through competition for the ribosome rather than through their translational products.

Importantly, uORF-mediated inhibition can be alleviated under a variety of conditions, permitting translation of downstream mORF. The well-studied mechanism underlying such a translational switch from uORF to mORF occurs in a few transcription factors, such as the yeast GCN4 and the mammalian ATF4, involving stress-induced phosphorylation and inactivation of eukaryotic translation initiation factor 2α (eIF2α). However, inactivation of eIF2a would lead to a global translational shutdown, which, while critical for some stress responses (for example, nutrient deprivation), is deleterious and absent during most eukaryotic developmental stages and under abiotic and biotic stress conditions (for example, immune responses in plants). This raises the fundamental question of what mRNA features, working in conjunction with the translational machinery, dynamically dictate which AUGs are chosen to initiate translation and thereby control protein production under different conditions.

### Example 1. Ribosome Occupancy on uORFs and mORFs is Dynamically Regulated During Pattern-Triggered Immunity

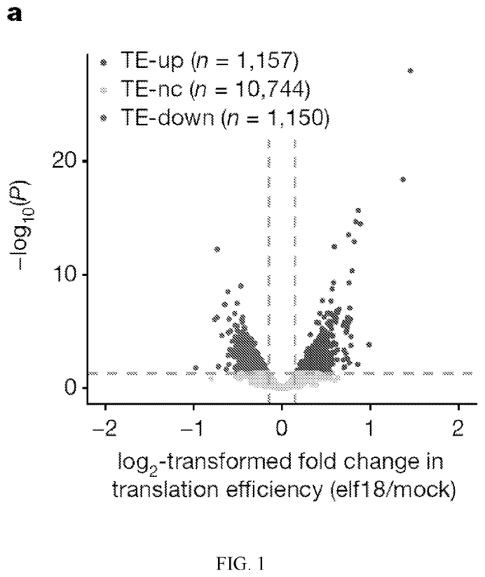

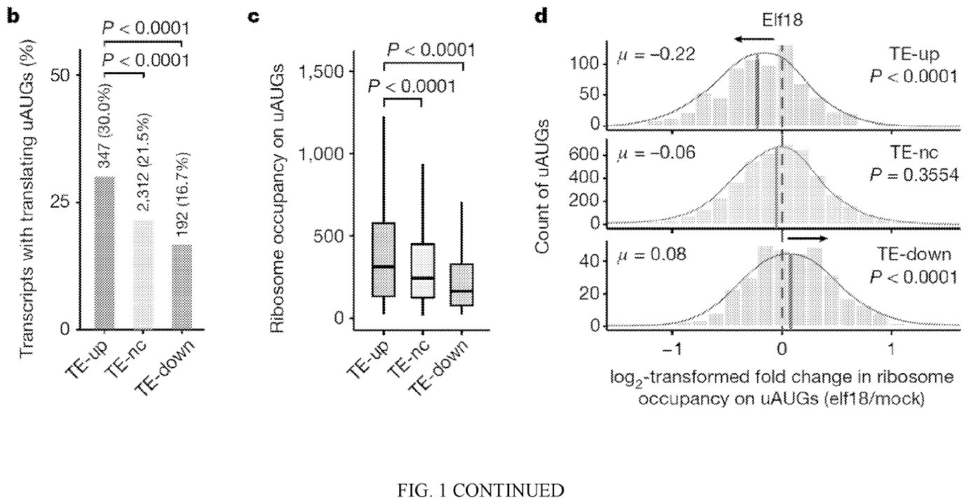

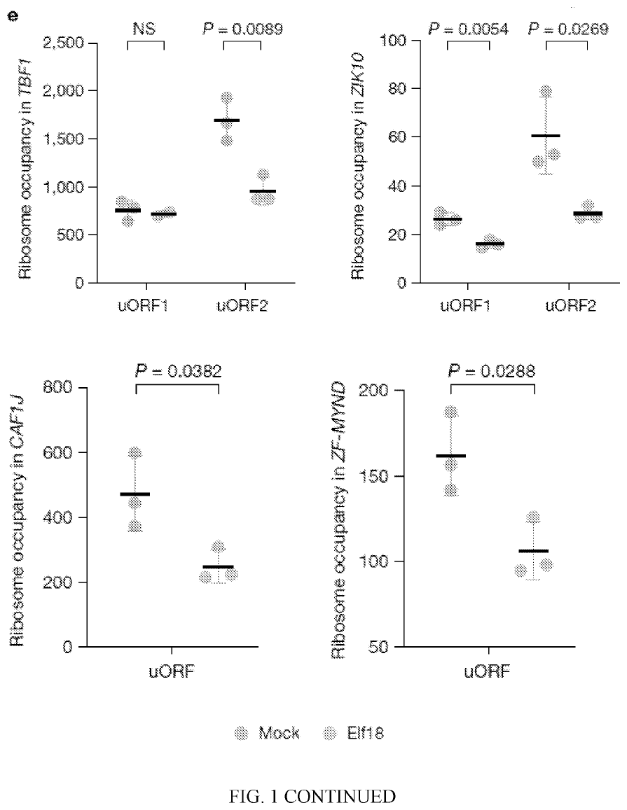

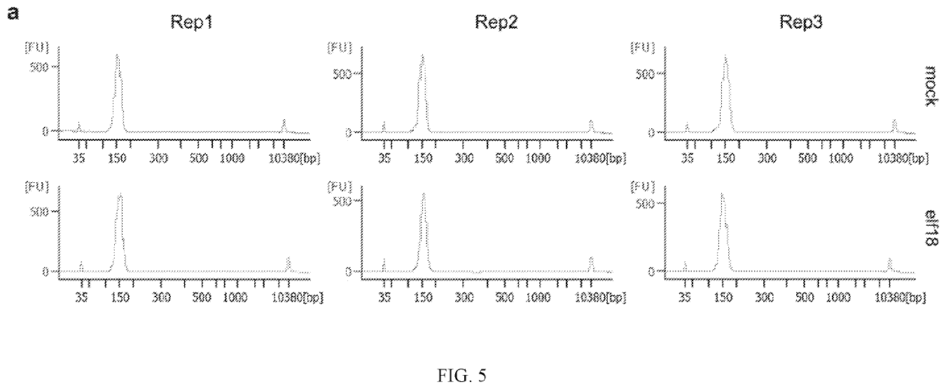

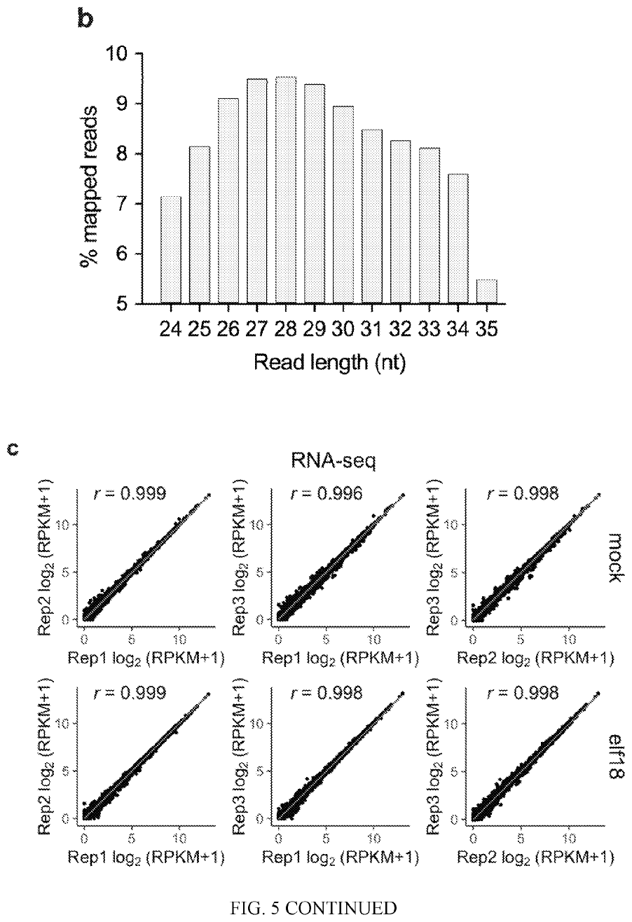

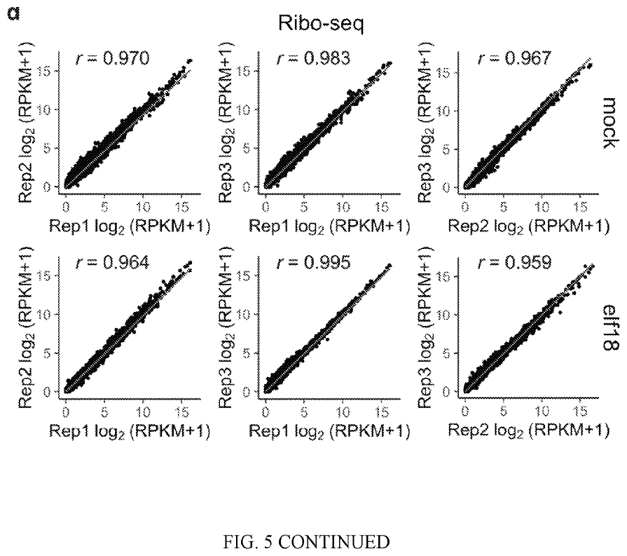

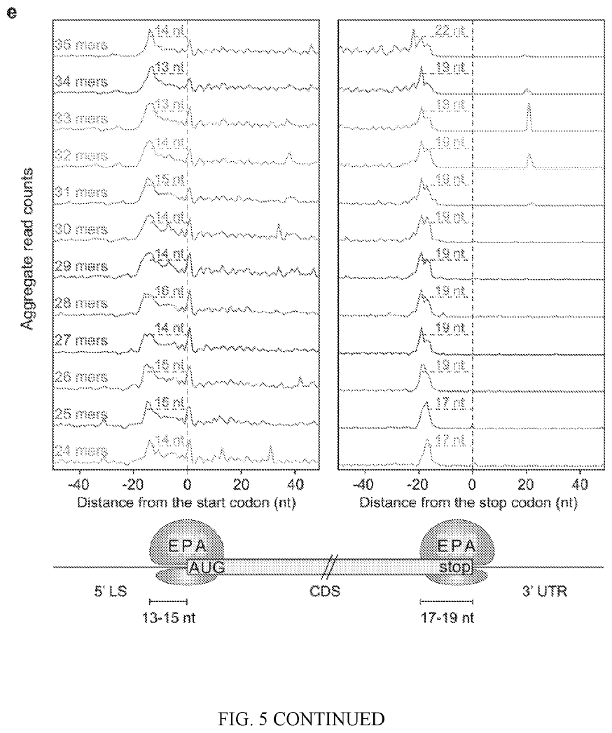

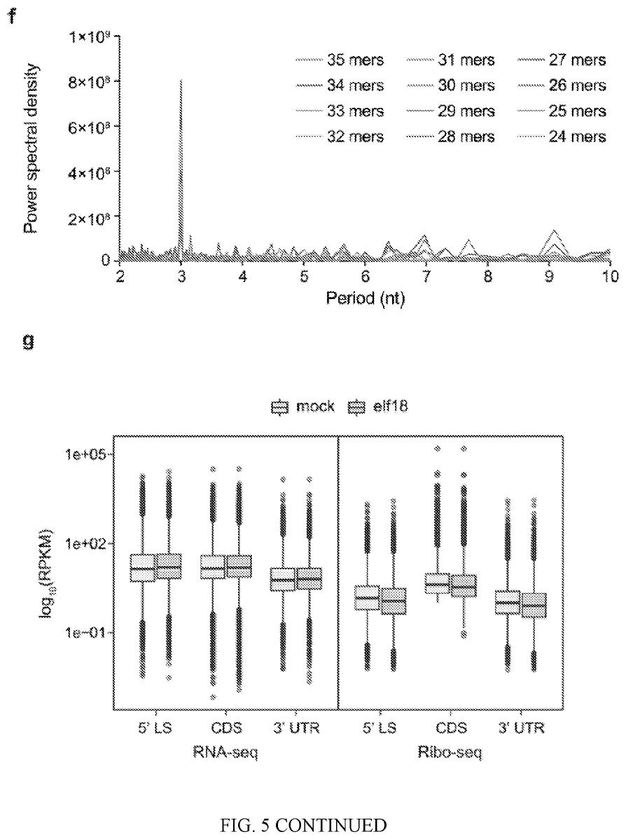

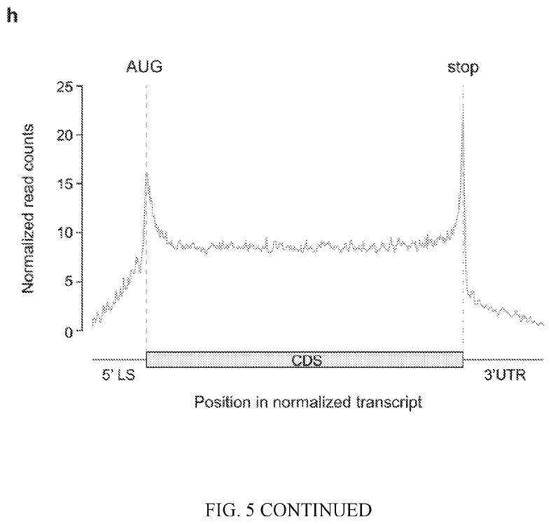

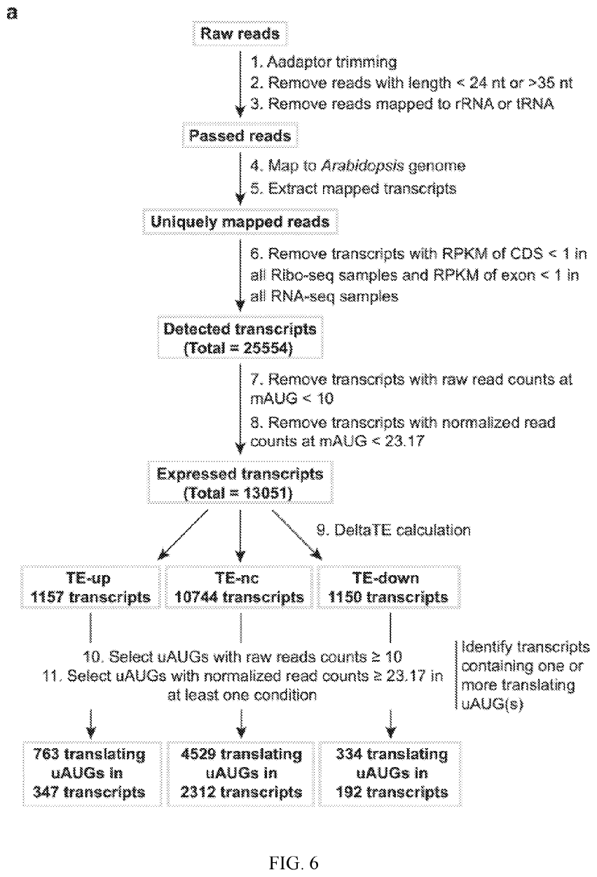

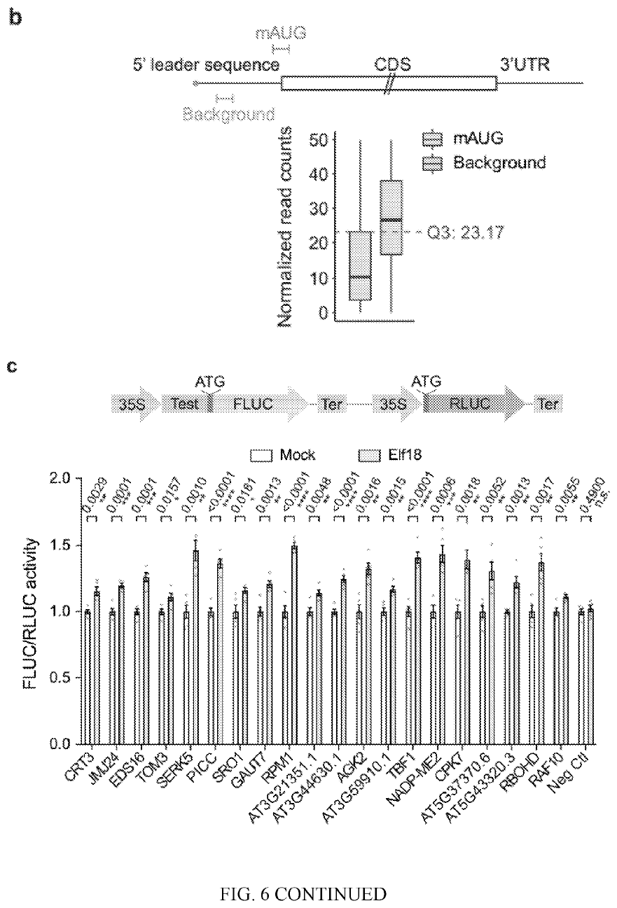

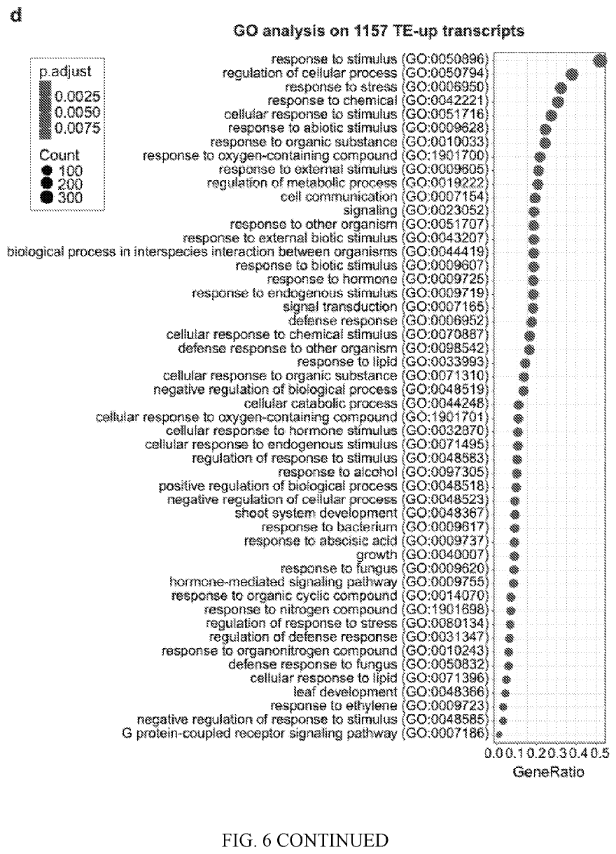

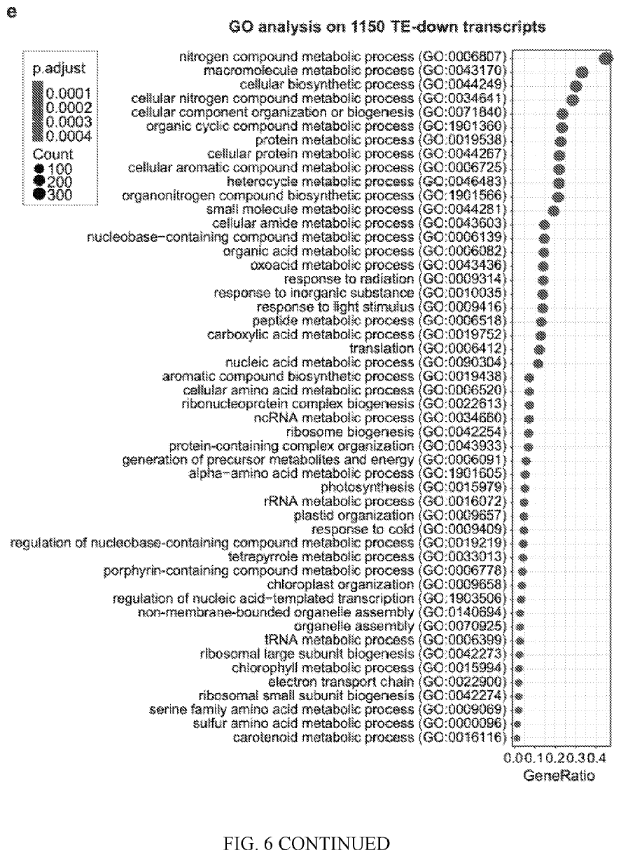

To identify novel mechanisms involved in uAUG-mediated translation regulation, global ribosome-sequencing (Ribo-seq, sequencing of ribosome-protected RNA fragments) was initially performed on Arabidopsis seedlings in response to the induction of pattern-triggered immunity by elf18 (N-terminal epitope of the bacterial elongation factor Tu). The optimized Ribo-seq pipeline had a sufficiently high resolution to examine the translational activities in 5′ leader sequences (see FIG. 5 and Methods for details). Comparing the elf18-treated samples to mock-treated controls, the inventors identified, among the 13051 expressed transcripts, 1157 with increased translational efficiency (TE-up), 1150 with decreased translational efficiency (TE-down), and the rest with no significant changes in translational efficiency (TE-nc) (FIG. 1, panel a, and FIG. 6, panels a, b). We selected 20 TE-up transcripts and used their 5′ leader sequences to drive the translation of the constitutively transcribed firefly luciferase (FLUC) reporter. Using the constitutively expressed Renilla luciferase (RLUC) as a control, this ‘dual luciferase’ assay22 confirmed the elf18-induced translation (FIG. 6, panel c) observed in the Ribo-seq results. Gene ontology (GO) analysis of the TE-up genes revealed an enrichment of biological processes in response to a variety of environmental stresses, such as biotic stimuli, abiotic stimuli, and chemicals, whereas GO terms for the TE-down genes are mostly growth-related metabolic processes (FIG. 6, panels c, d).

To systematically identify uAUGs that can be recognized by the preinitiation complex and initiate translation (“translating uAUGs”), the inventors focused on those uAUGs with ribosomal associations above the background levels (FIG. 6, panels a, b). 5626 translating uAUGs were identified across all the 13051 expressed transcripts, with some transcripts possessing multiple translating uAUGs. It was discovered that translating uAUGs were significantly enriched in the TE-up transcripts (30.0%), compared to the TE-nc (21.5%) and TE-down mRNAs (16.7%; FIG. 1, panel b). This finding suggests that translation initiation from uAUGs may have a general role in regulating immune-associated translation.

Next, global translational dynamics on the translating uAUGs were examined. Strikingly, under the mock condition, translating uAUGs in the TE-up transcripts had significantly higher ribosomal associations compared to those in the TE-nc and TE-down transcripts (FIG. 1, panel c), suggesting higher translation initiation rates from the uAUGs in the TE-up transcripts without immune induction (mock). In response to elf18 treatment, there was a significant decrease in ribosomal association with the translating uAUGs in the TE-up transcripts, whereas this reduction was not detected in the TE-nc and TE-down transcripts (FIG. 1, panel d). Closer examination of the Ribo-seq data on a few representative TE-up transcripts, including those from the marker gene TBF1, showed a significant reduction in ribosome occupancy on the inhibitory uORFs (uORF2 for TBF1 and ZIK10) in response to elf18 treatment (FIG. 1, panel e). As translation initiation from uAUGs typically inhibits the translation of downstream mORFs, this elf18-triggered reduction in uAUGs translation suggests an immune-induced release of uAUG-mediated inhibition on downstream mORF translation. Collectively, both the global characterization (FIG. 1, panels c, d) and direct analysis of marker genes (FIG. 1, panel e) revealed the common regulatory dynamics of translating uAUGs in the TE-up transcripts: they are preferentially recognized and translated under the mock condition, but are bypassed to permit translation initiation from mAUGs in response to immune induction.

### Example 2. Double-Stranded RNA Structures Downstream of AUGs Dictate their Selection for Translation Initiation

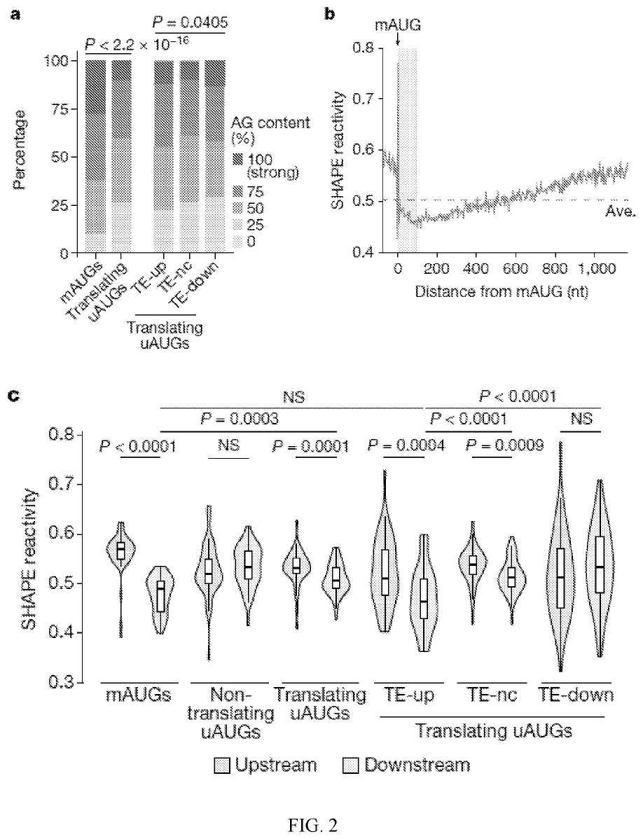

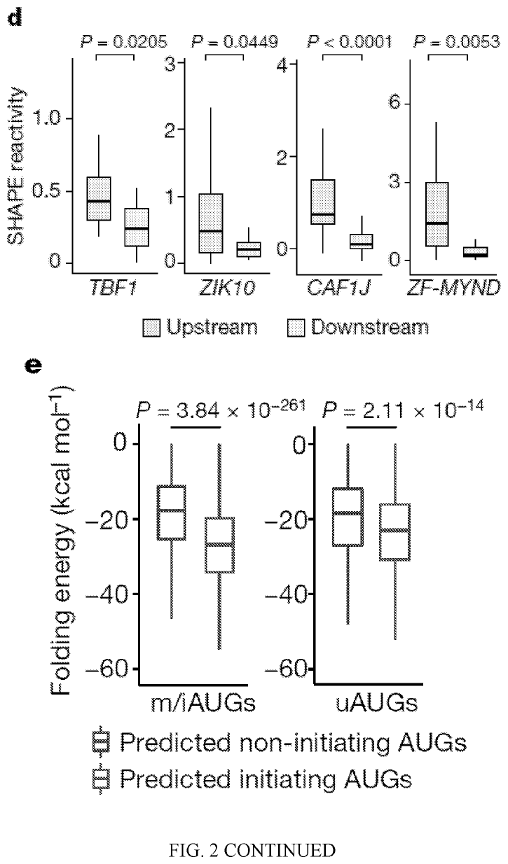

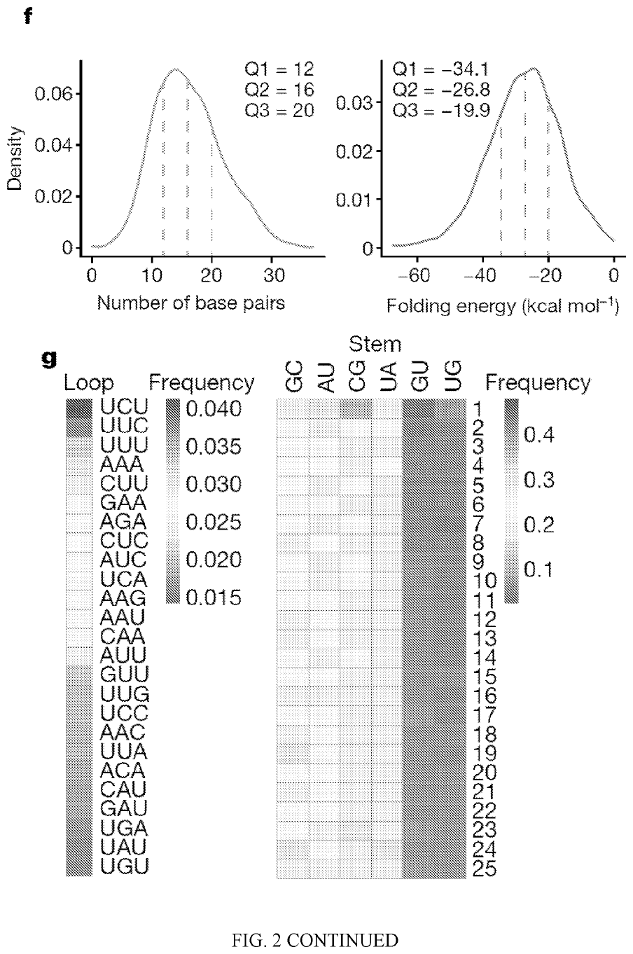

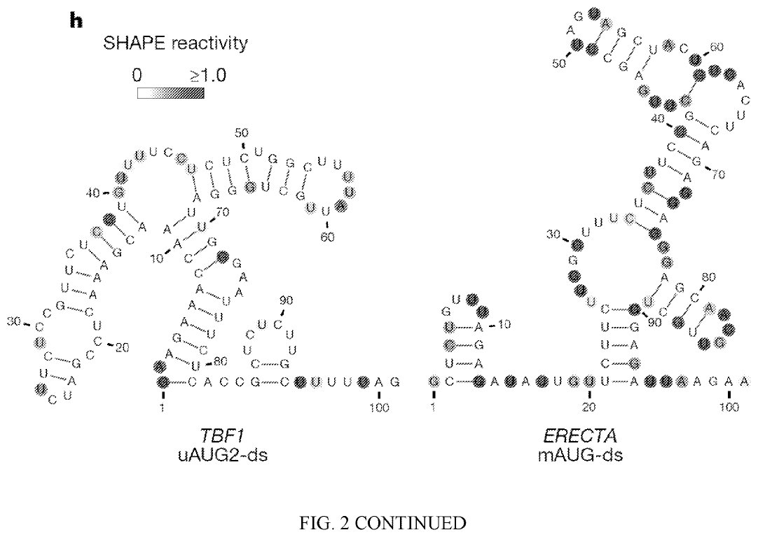

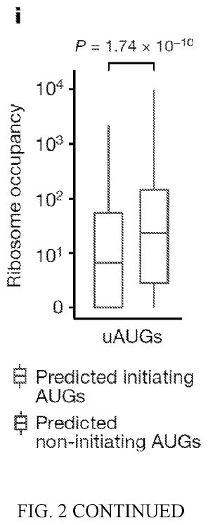

To address the question how start codons are dynamically selected to initiate translation under different conditions, the Kozak sequence context flanking the AUGs was first assessed, because this −3 to +4 region (with A in AUG being +1) could impact the start codon recognition by the translation preinitiation complex. Although the Kozak sequence context in plants has not been comprehensively defined, a previous analysis suggested that a higher adenine and guanine (ΔG)-content is associated with higher translational activity. Using this criterion, the Kozak contexts was assessed for the uAUGs and mAUGs in all the expressed transcripts. It was found that mAUGs have remarkably higher ΔG-contents than translating uAUGs (FIG. 2, panel a), in agreement with recent findings in animals that uAUGs tend to have less preferable Kozak sequence contexts compared to mAUGs. The Kozak contexts for translating uAUGs among the TE-up, TE-nc, and TE-down transcripts was next compared, and it was found that they have similar ΔG-contents (FIG. 2, panel a). This suggests that while the Kozak sequence context is important for start codon recognition under static conditions, it is unlikely to be responsible for the elf18-mediated switch from uAUG to mAUG translation in the TE-up transcripts.

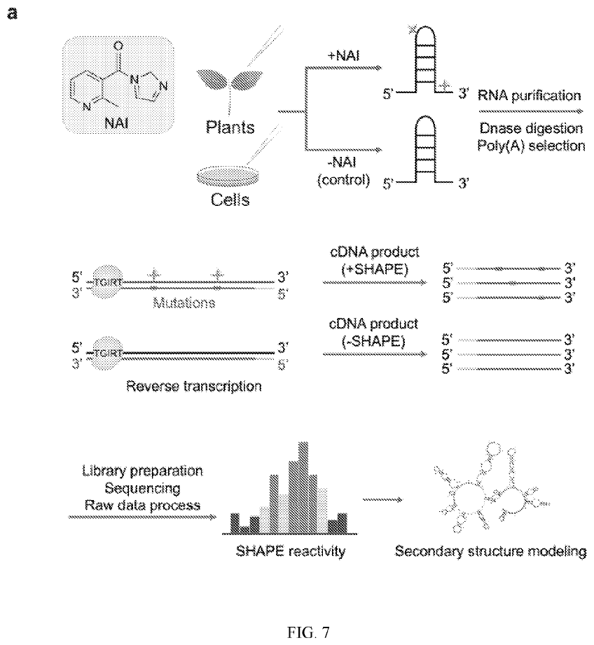

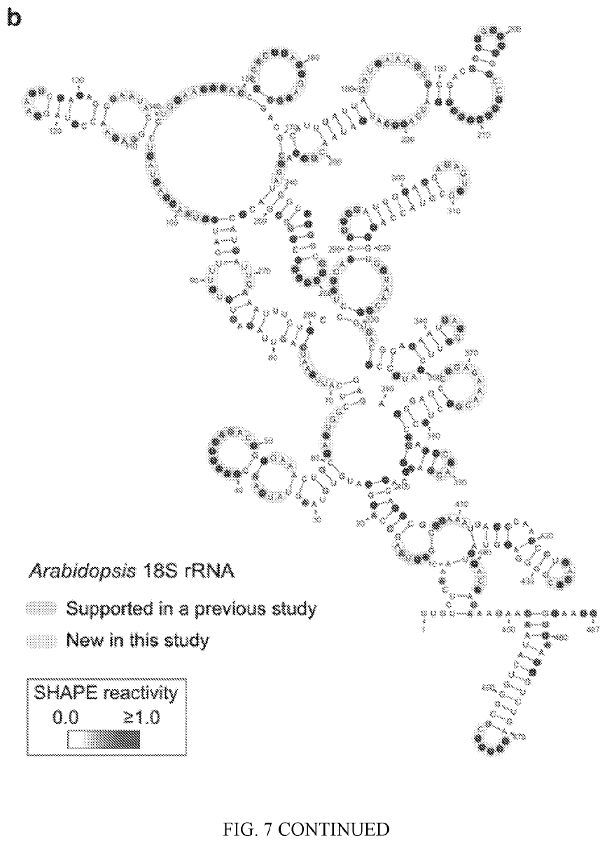

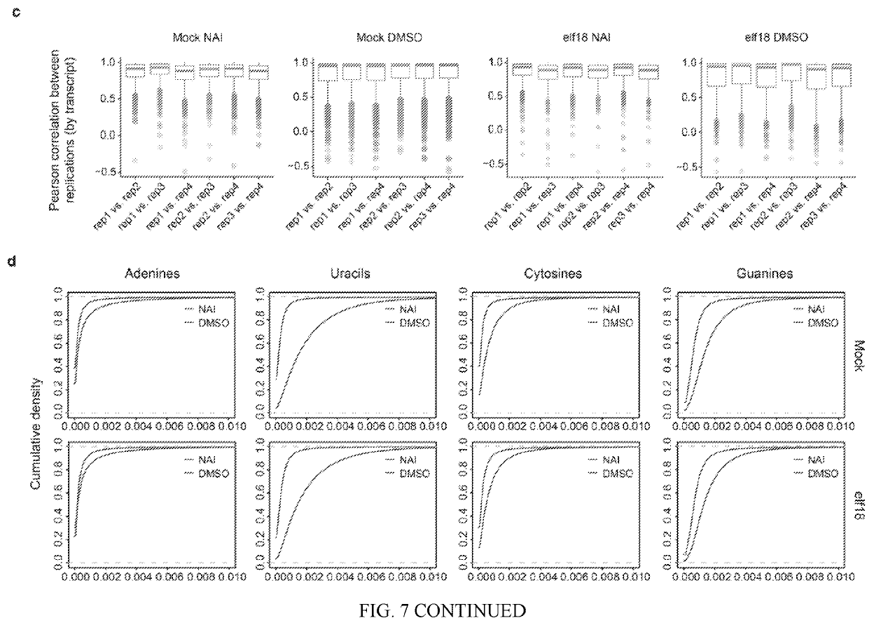

Beyond the primary sequences, a possible involvement of RNA secondary structures in this dynamic selection of translation start codons was next considered. To probe the in vivo RNA secondary structural dynamics, selective 2′-hydroxyl acylation and primer extension based on the mutational profile (SHAPE-MaP) was adapted to detect global in planta RNA secondary structural changes at nucleotide resolution with and without immune induction. This strategy relies on SHAPE reagents (here, 2-methylnicotinic acid imidazolide, NAI), a group of hydroxyl-selective electrophiles that react with the 2′-hydroxyl position of unpaired residues of RNA regardless of whether they are associated with RNA-binding proteins. The resulting 2′-O-adducts cause mutations in the cDNA during reverse transcription, which are detected through sequencing to create SHAPE reactivity profiles, yielding quantitative measurements of RNA structures (FIG. 7, panel a). Regions with lower SHAPE reactivity are those with higher levels of double-stranded RNA structures. To first validate the protocol, targeted in planta SHAPE-MaP of the Arabidopsis 18S rRNA was performed, and the signal obtained is not only consistent, but also significantly improved from that reported previously (FIG. 7, panel b).

The global in planta SHAPE-MaP analysis of mRNAs in Arabidopsis seedlings in response to mock or elf18 treatment was then performed. The quality of the data was supported by a strong correlation among the data obtained from all four replicates under each of the conditions (FIG. 7, panel c) and an overall higher mutation rate in all four nucleotides in the NAI-modified samples than that in the unmodified control samples (FIG. 7, panel d). For subsequent analyses, only data that passed the stringent cut-offs for read depth and completeness were used to enable accurate structure modelling (see Methods for details).

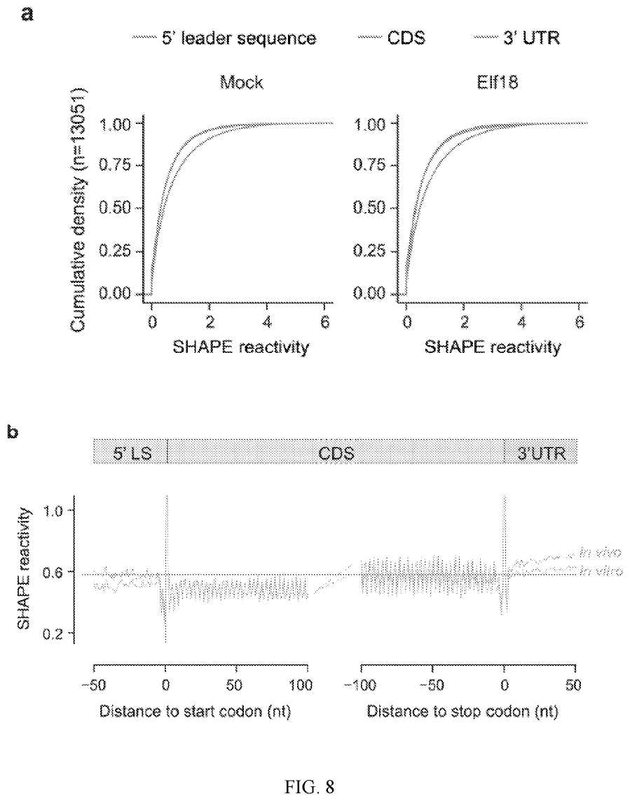

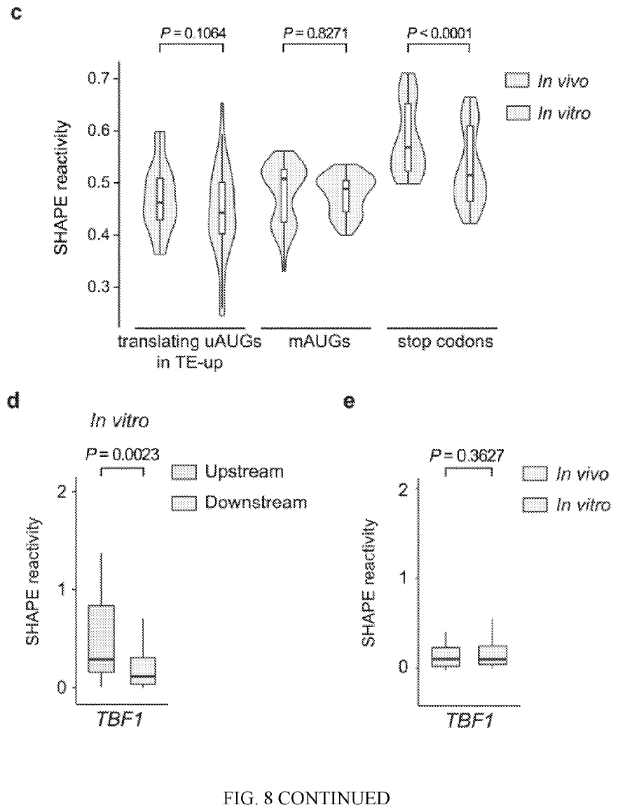

From the global SHAPE-MaP data, it was found that 5′ leader sequences and coding sequences (CDSs) had higher levels of double-stranded structures than 3′ untranslated regions (3′ UTRs) across all expressed transcripts, depicted by their lower cumulative SHAPE reactivities (FIG. 8, panel a). Similar to the observations in other RNA structuromic studies, there was an extraordinarily high average SHAPE reactivity at the mAUGs and the stop codons of CDSs in all expressed transcripts (FIG. 2, panel b).

Interestingly, it is noted that although the overall SHAPE reactivities of the 5′ leader sequences and the CDSs are comparable, nucleotides immediately downstream of the start codons had noticeable lower SHAPE reactivities (FIG. 2, panel b), indicating higher levels of double-stranded structures in this region. It was questioned whether this structural feature is related to start codon recognition and translation initiation from mAUGs, and whether a similar feature exists for uAUGs. To answer these questions, the SHAPE reactivity for each of the 50 nucleotides (nt) upstream and downstream of AUGs was first examined to determine whether there is a statistically significant difference. It was found that under the mock condition, nucleotides downstream of mAUGs and translating uAUGs had significantly lower SHAPE reactivities compared to those upstream, but this was not observed for non-translating uAUGs (FIG. 2, panel c). It was further investigated whether the observed structural feature may also contribute to the dynamic regulation of uAUG-mediated translation in the TE-up, TE-nc, and TE-down transcripts (FIG. 1, panels c, d). Interestingly, it was discovered that under the mock condition, translating uAUGs in the TE-up transcripts had significantly lower SHAPE reactivities (i.e., more double-stranded structures) in their downstream regions compared to those in the TE-nc and TE-down transcripts (FIG. 2, panel c), with several representatives shown in FIG. 2, panel d.

To assess the possibility that the low SHAPE reactivity that was found downstream of mAUGs and translating uAUGs was a result of association with ribosomes or RNA-binding proteins, we performed global in vitro SHAPE-MaP experiments on the same samples in the mock condition. The overall SHAPE reactivities in vitro were lower than those observed in vivo, suggesting a lower degree of single-strandedness in vitro (FIG. 8, panel b). We found that in the absence of proteins, the overall SHAPE reactivities in regions immediately downstream of mAUGs and translating uAUGs in the TE-up transcripts were not significantly changed from those obtained from the in vivo SHAPE-MaP (FIG. 8, panels b,c), indicating that the low SHAPE reactivities observed in this region are unlikely to be due to protein binding, but are more likely to be attributed to double-stranded RNA (dsRNA) secondary structures. Hence, we named these structures downstream of mAUGs and uAUGs ‘mAUG-ds’ and ‘uAUG-ds’, respectively. Targeted in vitro SHAPE-MaP analysis of the TE-up marker transcript TBF1 also showed that the removal of proteins had no significant effect on the SHAPE reactivity patterns in its uAUG2-ds region (FIG. 8, panels d,e).

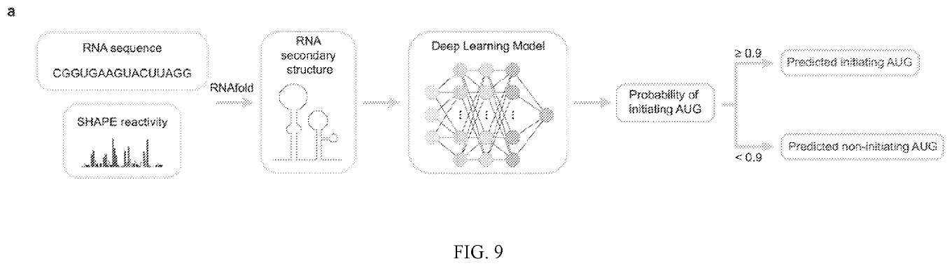

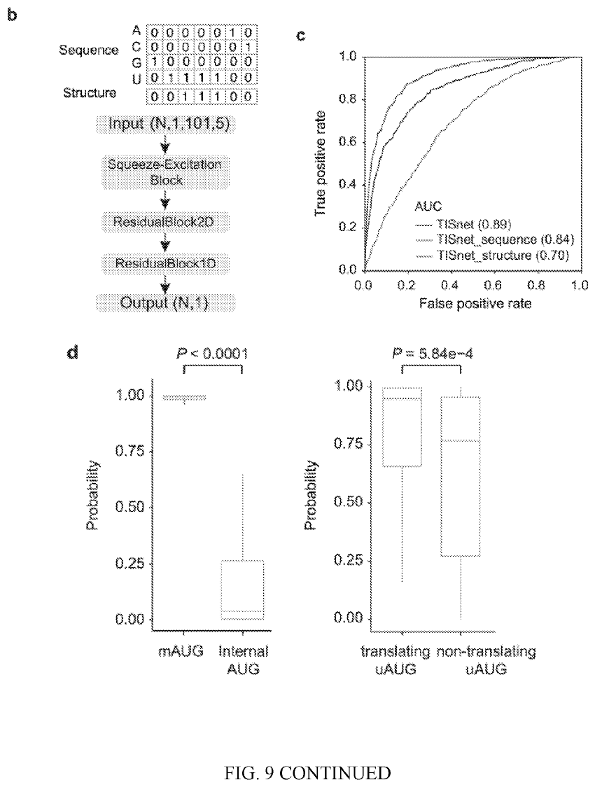

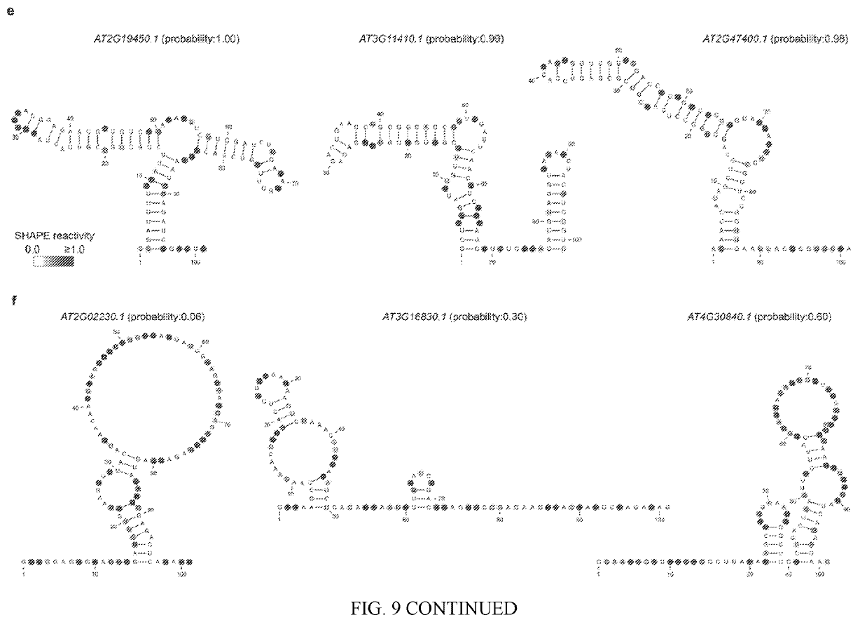

To characterize the structural patterns that contribute to mAUG and uAUG selection, Translation Initiation Site prediction using deep neural network (TISnet) was developed, which integrates primary sequences and the SHAPE-MaP data to predict translation initiation sites. To train the TISnet model, data from mAUGs and internal AUGs were used as positive and negative samples, respectively (see Methods for details). AUGs with high probability (≥0.9) are classified as predicted initiating AUGs (FIG. 9, panels a, b). The good prediction performance of TISnet is shown by the high Area Under the receiver operating characteristic Curve (AUC) score of 0.89 (FIG. 9, panel c) and the clear differences between the predicted probabilities of mAUGs and internal AUGs and between translating uAUGs and non-translating uAUGs (FIG. 9, panel d).

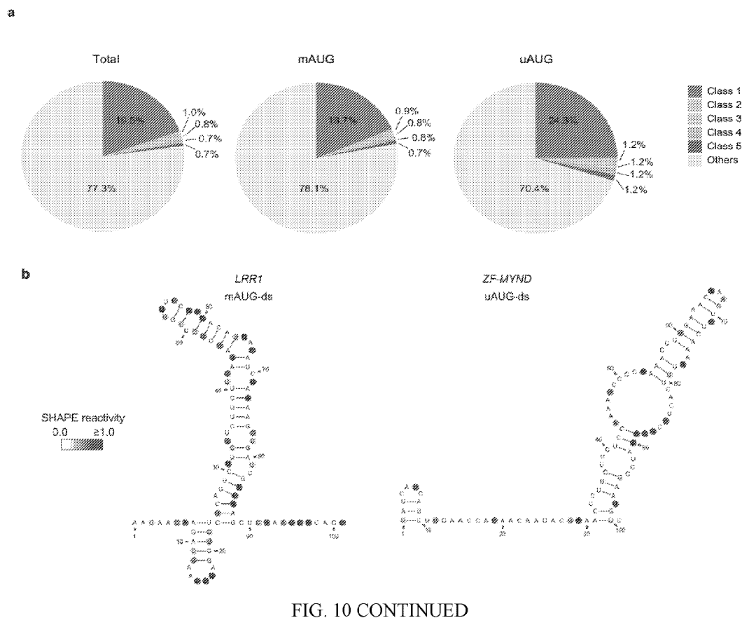

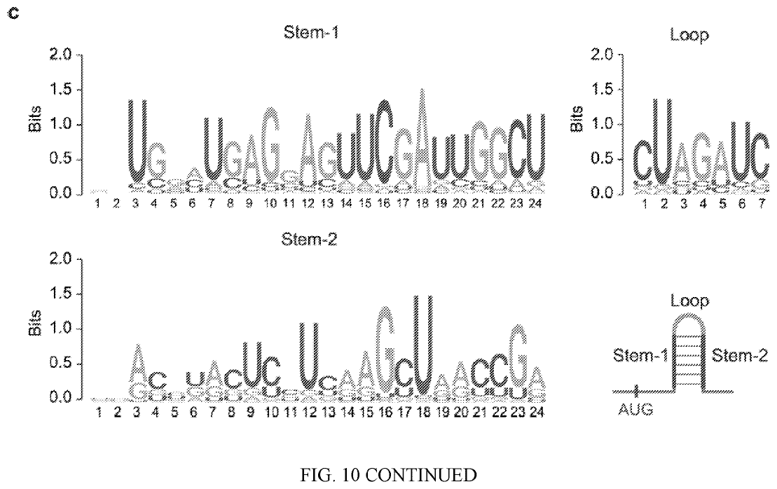

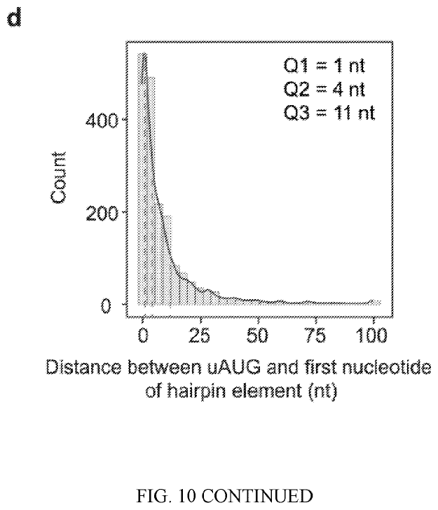

Our model further indicated that double-stranded RNA structures downstream of mAUGs (“mAUG-ds”) and uAUGs (“uAUG-ds”) are responsible for the start codon selection for translation initiation because downstream regions of both predicted initiating mAUGs and uAUGs had significantly lower folding energy and more double-stranded structures than predicted non-initiating AUGs (FIG. 2, panel e, FIG. 9, panels e, f). Majority of these mAUG-ds and uAUG-ds have the folding energies ranging from −34.1 kcal/mol to −19.9 kcal/mol and the numbers of base pairs in the stems from 12 to 20 (FIG. 2, panel f) with the nucleotides GC and AU pairs most prevalently present in the stems and UCU and CUG in the loops (FIG. 2, panel g). Hierarchical clustering on these elements according to the sequence similarities within loops and stems was performed and the largest class (class 1) contains mAUG-ds and uAUG-ds in 341 of 1746 transcripts (19.5%), including TBF1, ERECTA, LRR1, and ZF-MYND (FIG. 2, panel h, FIG. 10). Moreover, most of the double-stranded structures begin within 25 nt downstream of uAUGs (FIG. 10, panel d).

Next, the application of TISnet in predicting translating uAUGs was considered by examining the translational activity of the predicted initiating uAUGs and non-initiating uAUGs using the disclosed Ribo-seq data. A significant higher ribosome occupancy was found on the predicted initiating uAUGs than the non-initiating uAUGs, suggesting that TISnet can be used to accurately predict potential initiating uAUGs with translation activities (FIG. 2, panel i).

The pervasive presence of uAUG-ds in the TE-up transcripts is likely to contribute to the translation inhibitory roles of uAUGs under normal conditions, because downstream structures could slow the scanning of the translation preinitiation complex to enhance the chance of whole ribosome assembly and initiate translation from uAUGs instead of mAUGs. It is worth emphasizing that in contrast to previously reported upstream secondary structures which inhibit translation, the double-stranded RNA structures downstream of mAUGs and uAUGs identified in the study promote translation initiation.

### Example 3. UAUG-Ds Regulate Translation in Plants and in Human Cells

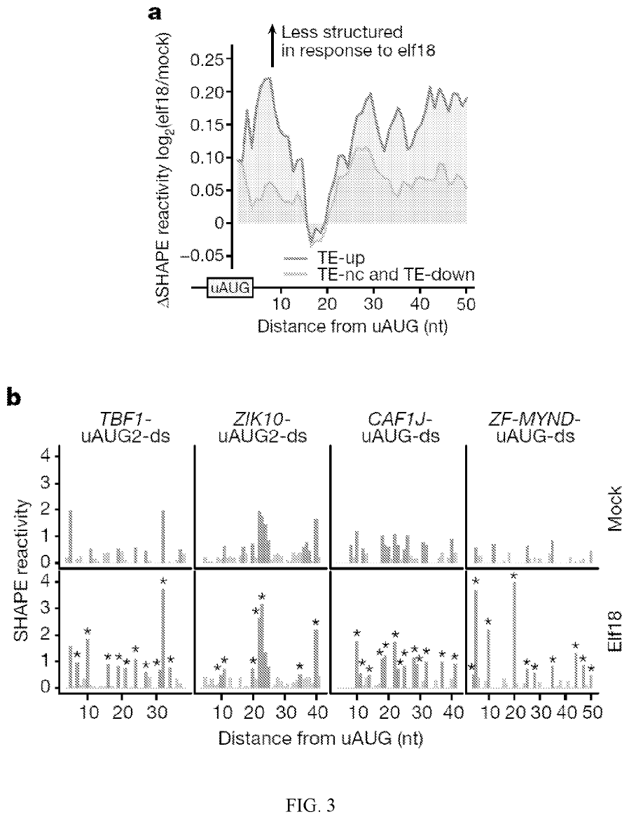

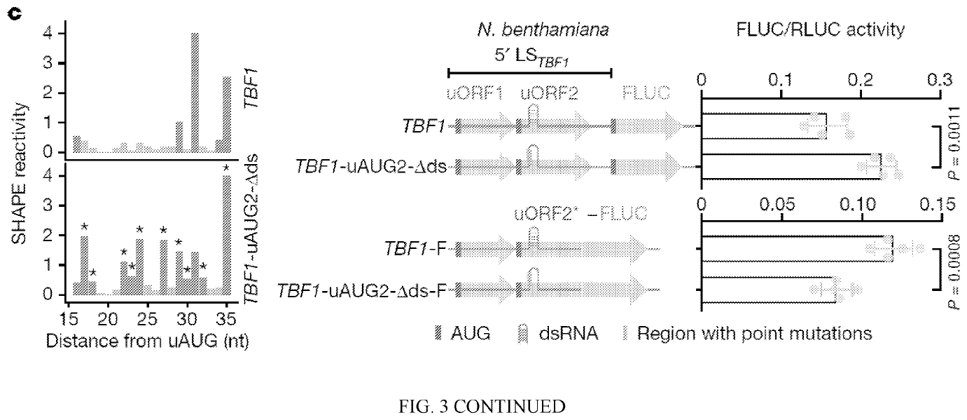

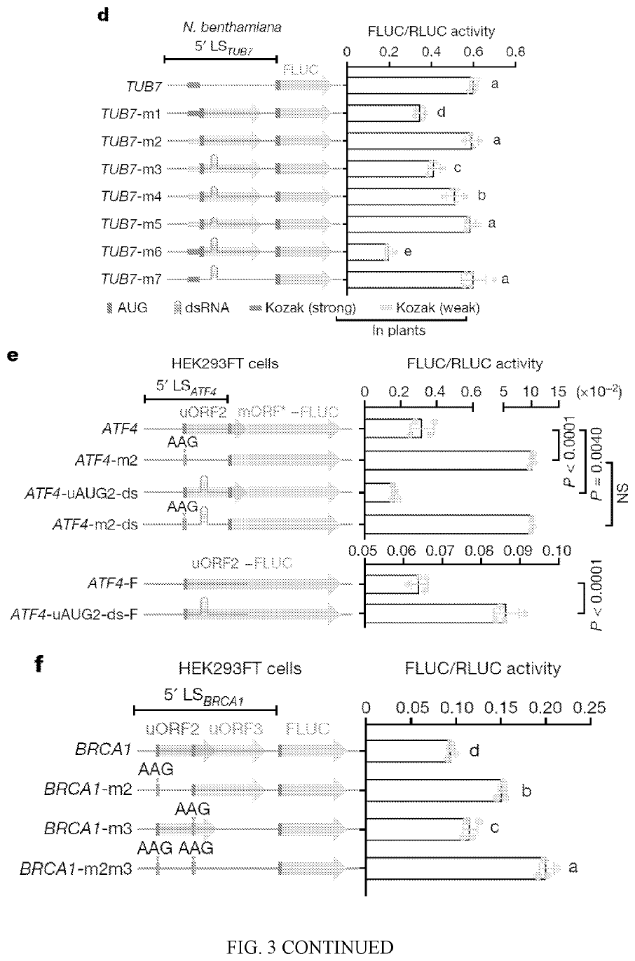

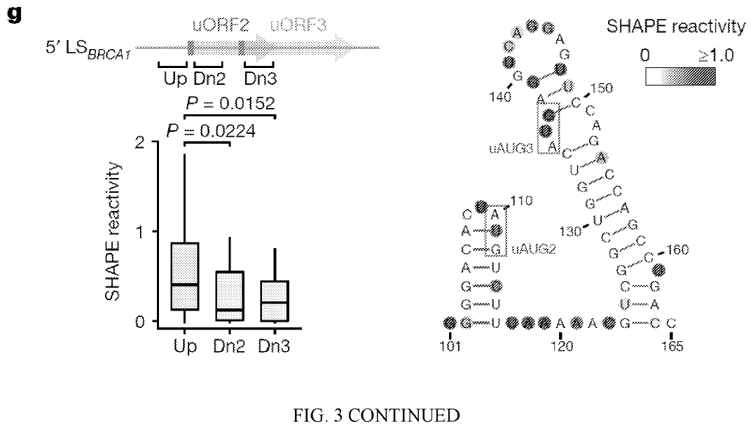

Since the Ribo-seq data revealed an elf18-triggered shift in translation from uORFs to mORFs in the TE-up transcripts (FIG. 1, panels c, d), it was hypothesized that this global translational reprogramming is regulated by changes in uAUG-ds. Indeed, a significant elf18-induced increase of SHAPE reactivities in the uAUG downstream regions (FIG. 3, panel a) was observed, suggesting that uAUG-ds are resolved in response to the immune induction. More importantly, the extent of the change is much bigger in the TE-up transcripts than in the TE-nc and TE-down transcripts (FIG. 3, panel a). Closer examination of the representative TE-up transcripts confirmed the global observation (FIG. 3, panel b). It is proposed that the immune-induced reduction in uAUG-ds complexity allows the preinitiation complex to scan beyond the uAUGs to initiate translation from downstream mAUGs.

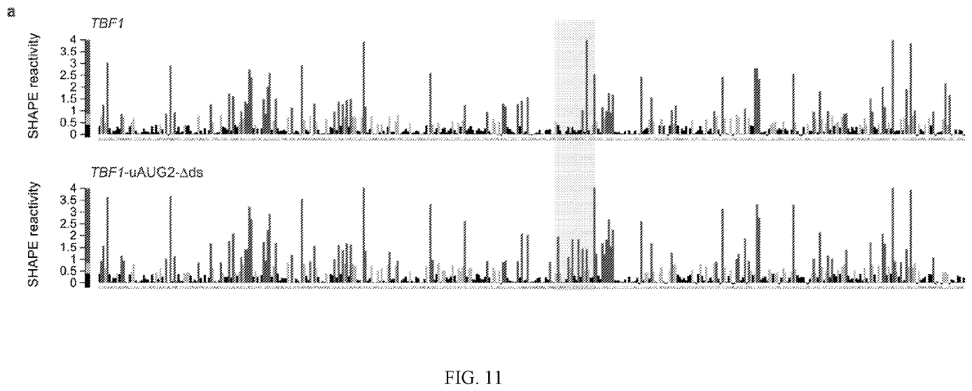

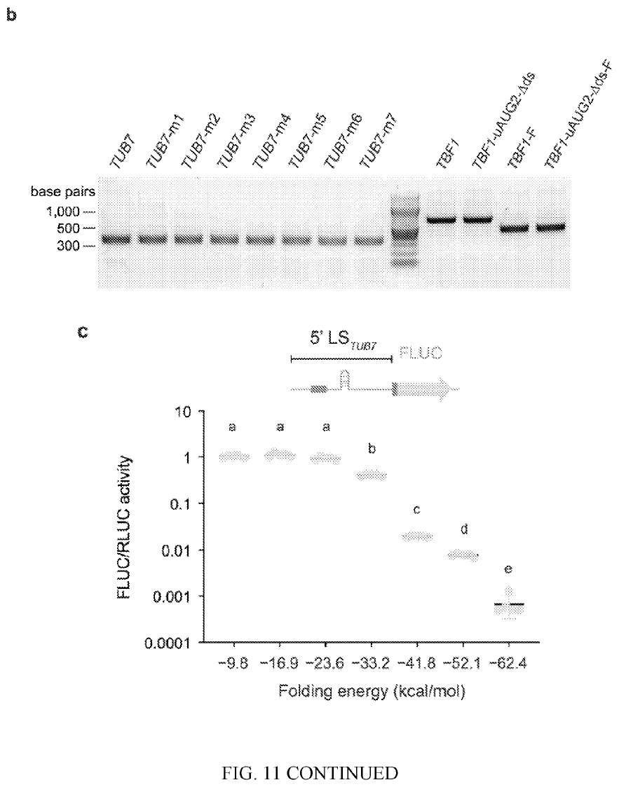

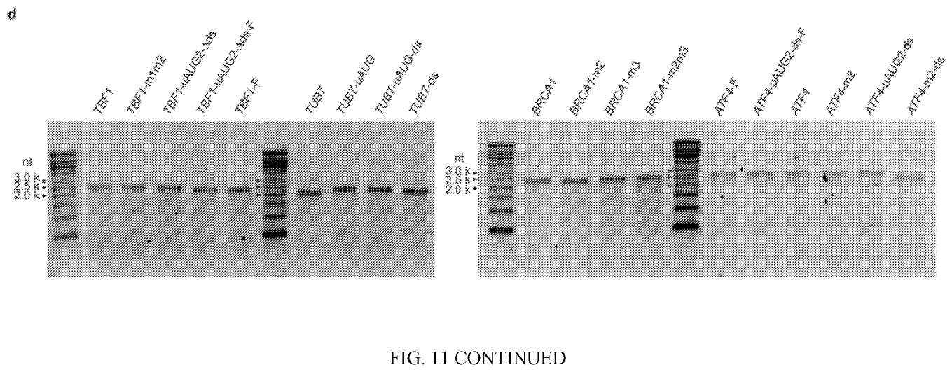

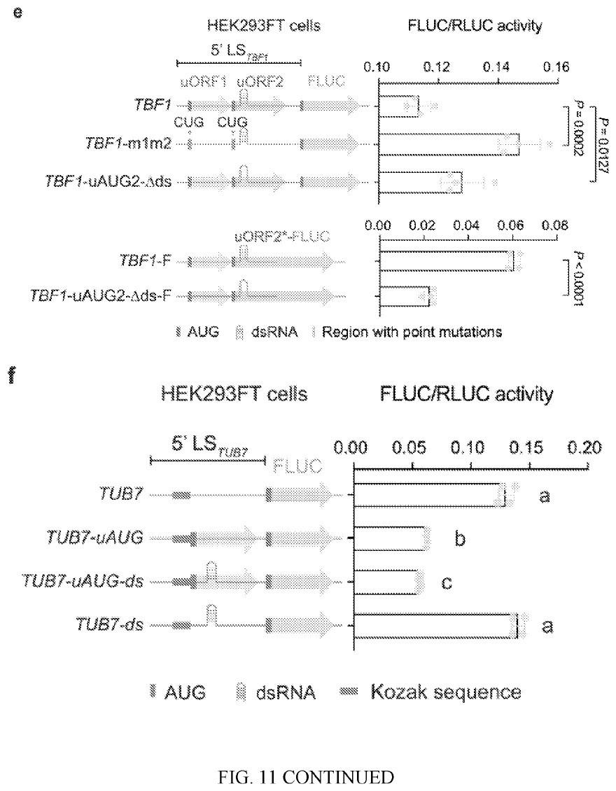

To validate the role of uAUG-ds in dynamically dictating start codon selection and thus regulating downstream protein production, uAUG-ds in the TBF1 transcript were first examined, using a dual-luciferase reporter system which was transiently expressed in Nicotiana benthamiana. In this system, translation of the firefly luciferase reporter (FLUC) is driven by the TBF15′ leader sequence, and the resulting reporter activity is normalized to that of the constitutively expressed Renilla luciferase (RLUC) on the same construct. When the hairpin structure was removed by introducing point mutations in the uAUG2-ds in the TBF15′ leader sequence (TBF1-uAUG2-Δds) to mimic the structural opening in response to elf18 (FIG. 3, panel c, left and FIG. 11, panel a), a significant increase in the FLUC/RLUC activity was observed (FIG. 3, panel c, right). The role of uAUG2-ds in enhancing translation initiation from uAUG2 was further supported using another reporter in which FLUC is fused in-frame with uAUG2 instead of mAUG (FIG. 3, panel c, right). Altogether, these results demonstrate that a double-stranded structure downstream of uAUGs (uAUG2 for the TBF1 transcript) is conducive to uORF-mediated inhibition of downstream mORF translation by facilitating translation initiation from the uAUGs. This inhibition may be alleviated during stress when the RNA double-stranded structure is unwound to allow the translation preinitiation complex to scan beyond uAUGs to initiate mORF translation.

To show that the dynamic function of uAUG-ds in regulating translation initiation is generalizable, we engineered a reporter using the naïve 5′ leader sequence of the Arabidopsis TUB7 (tubulin beta-7) gene to drive the translation of FLUC in the dual-luciferase reporter system. We then mutagenized the 5′ leader sequence, without changing its length (FIG. 11, panel b), to introduce a uAUG in a strong or a weak Kozak context with or without artificial dsRNA structures (FIG. 3, panel d). The resulting reporter activities showed that in addition to the Kozak sequence context, the uAUG-ds structures within the optimal range (that is, 12-20 base pairs; −19.9 to −34.1 kcal mol-1 in FIG. 2, panel f) enhanced the recognition of uAUG for translation initiation and consequently dampened downstream reporter translation (FIG. 3, panel d). However, in the absence of the uAUG, the structure alone did not inhibit downstream reporter translation (FIG. 3, panel d, TUB7-m7), as long as it was within the optimal range of folding energy (FIG. 11, panel c), further supporting the role of uAUG-ds in engaging the ribosome to initiate translation from uAUGs.

To test whether the uAUG-ds-mediated translation initiation occurs in animals, we expressed the in-vitro-transcribed synthetic TUB7 reporter mRNAs and the Arabidopsis TBF1 reporter mRNAs in human HEK293FT cells (FIG. 11, panel d), and found that uORF-mediated reporter translation was most inhibited when uAUG-ds was present (FIG. 11, panels e, f). This result indicates that dsRNA enhances uAUG translation initiation in both plants and a human cell line. This conclusion was further supported when we introduced a dsRNA structure downstream of the uAUG2 in in-vitro-transcribed ATF4, a well-known mammalian stress-responsive gene13. This further inhibited the translation of ATF4 through enhanced translation initiation from the uAUG2 (FIG. 3, panel e, and FIG. 11).

It was then shown that uAUG-ds structures are present in mammalian transcripts by performing in vivo SHAPE-MaP analysis on the BRCA1 mRNA, a mutant version of a tumor suppressor transcript found in breast cancer tissue whose translation is known to be inhibited by uAUG2 and uAUG3 (FIG. 3, panel f). Significantly lower SHAPE-MaP activities were detected downstream of uAUG2 and uAUG3 compared to their upstream regions (FIG. 3, panel g), further that uAUG-ds can be a universal mechanism for dynamic start codon selection for translation initiation.

### Example 4. Inducible RNA Helicases Unwind uAUG-Ds to Alleviate Inhibition on mORF Translation

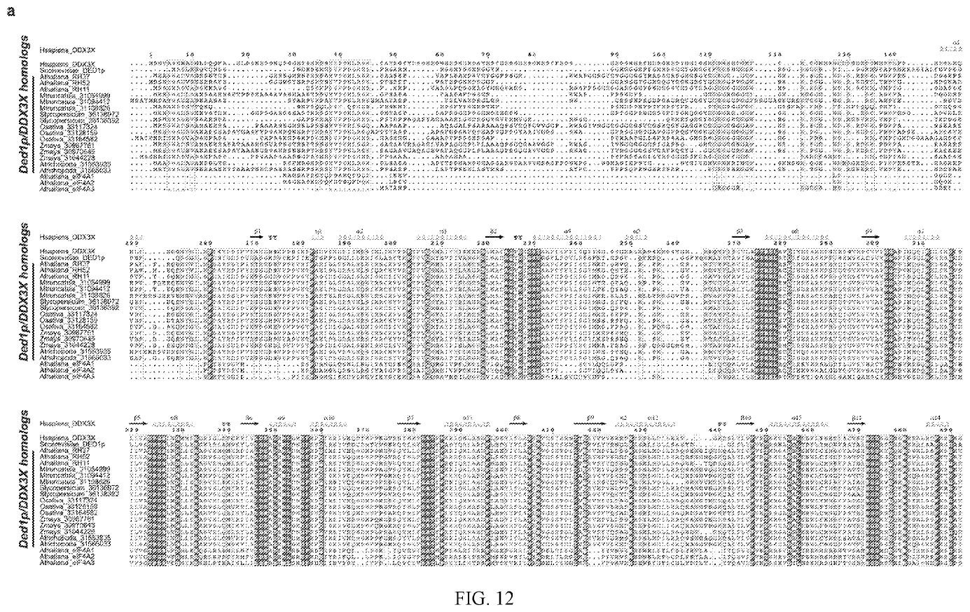

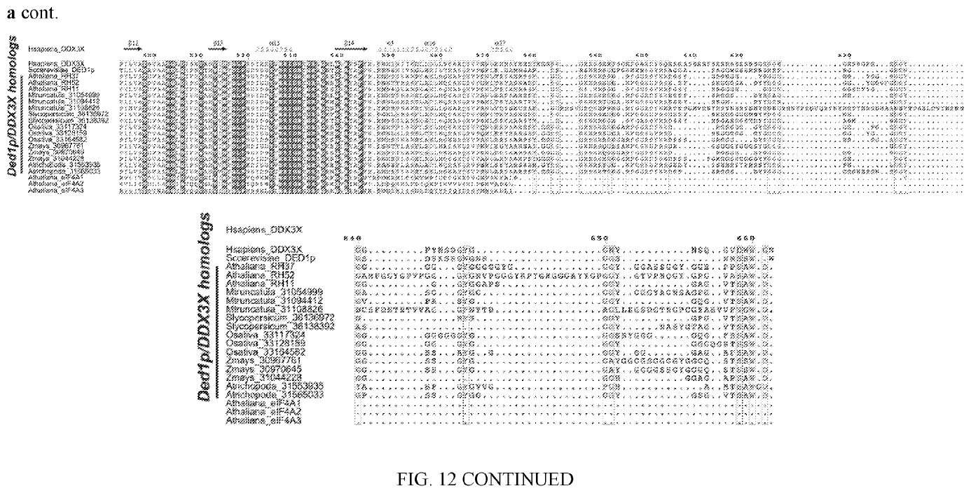

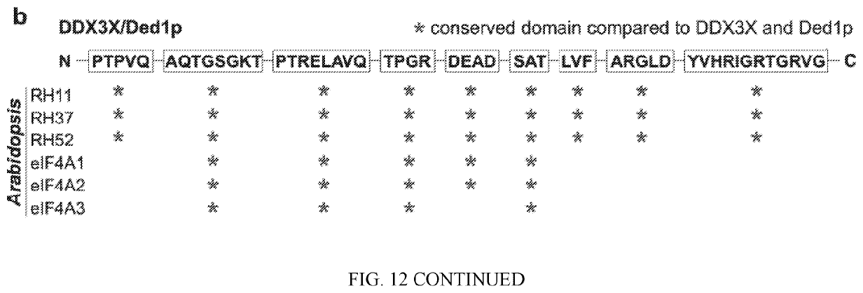

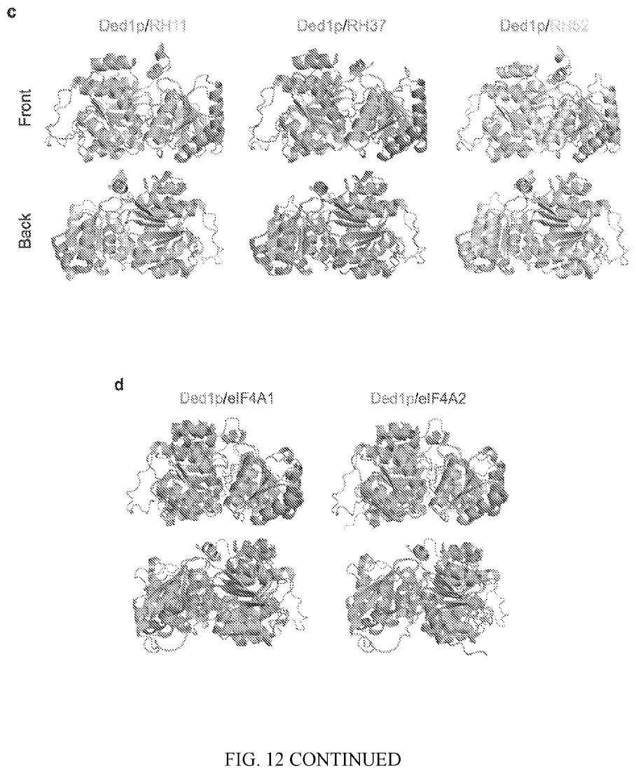

The next question was: How is uAUG-ds unwound to facilitate immune-induced translation in plants?Previous studies have suggested that some DEAD-box RNA helicases can serve as alternatives to the canonical eukaryotic translation initiation factor 4A (eIF4A) in binding and unwinding RNA for translation. To identify potential candidates for elf18-induced unwinding of uAUG-ds, the translational efficiency changes of the 54 known RNA helicases in Arabidopsis was examined and four candidates showing significant translational induction in response to elf18 were found (FIG. 4, panel a). Among them, only RH37 was predicted to be localized in the cytoplasm. A genome-wide homology analysis across angiosperms revealed another two close RH37 homologues, RH11 and RH52 (FIG. 12, panel a), consistent with the result of a recent gene family study. The translational inducibility by elf18 treatment was confirmed for RH37 and RH11 using the dual-luciferase reporter in which the 5′ leader sequences of these helicase transcripts were used to drive the FLUC translation (FIG. 4, panel b). Moreover, through comparisons of protein primary sequences, functional domains and structures predicted by AlphaFold, it was found that RH11, RH37, and RH52 are orthologous to the yeast Ded1p and the human DDX3X (FIG. 12, panels b-d). The sequence and structural homology to the yeast Ded1p also aligns well with the anticipated function for RH11, RH37 and RH52, because the yeast Ded1p, which functions with other initiation factors in the preinitiation complex, is required to unwind highly structured regions in 5′ leader sequences during translation initiation. Consistently, a recent study revealed that the Arabidopsis RH11 interacts with translation initiation factors. In addition, mutating the yeast Ded1p helicase activity causes enhanced translation initiation from near-cognate start codons upstream of structured regions. It was hypothesized that, opposite to the helicase mutant, immune-induced increases in RH11, RH37 and RH52 levels and/or activities may promote the unwinding of uAUG-ds, thus alleviating the uAUG-inhibition on mORF translation.

To test the disclosed hypothesis, the constructs Dex:RH37-YFP and Dex:RH11-YFP were built to put the transcription of RH37-YFP and RH11-YFP under the control of a dexamethasone (dex)-inducible system and transiently coexpressed each with the dual-luciferase reporter driven by the 5′ leader sequence of TBF1 in N. benthamiana. Strikingly, a significant increase was observed in the FLUC activities four hours after treatment with dex (FIG. 4, panel c). This suggests that a transient increase in the expression of these RNA helicases could lead to enhanced translation of TBF1.

It was next shown that the effect of these helicases is through remodeling of uAUG-ds because it was only observed when RH37 was coexpressed with the synthetic TUB7 reporter that contains uAUG-ds (FIG. 4, panel d). This result once again demonstrates that uAUG-ds can serve as a molecular switch to dynamically regulate translation initiation.

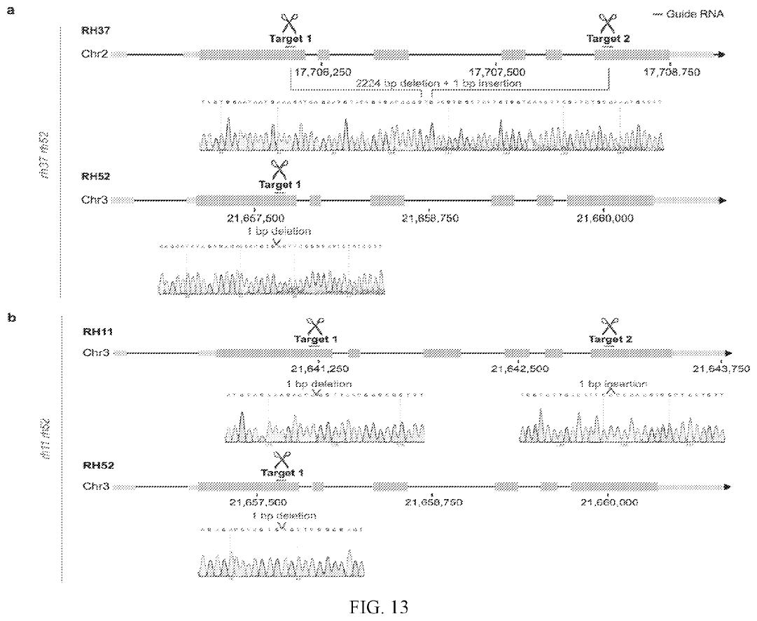

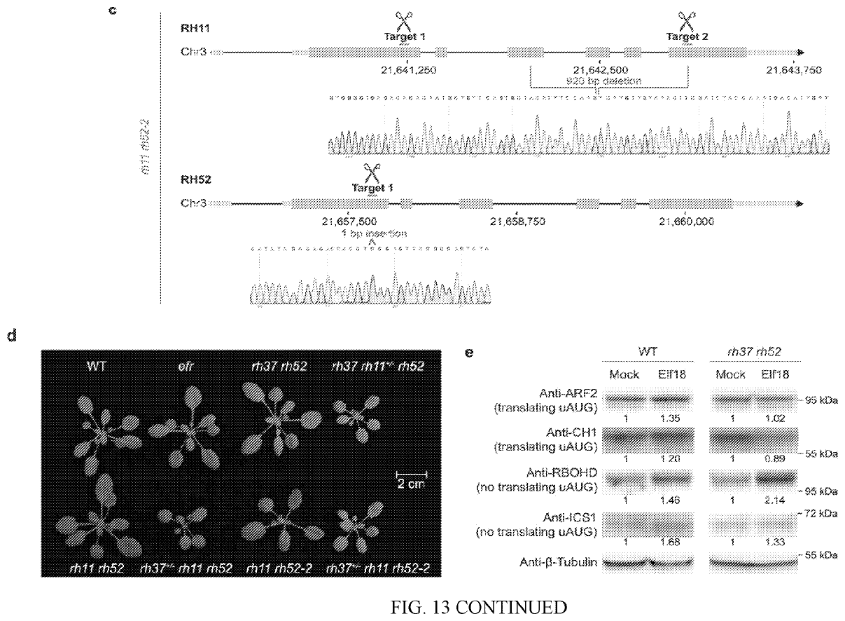

Finally, to confirm the roles of RH11, RH37, and RH52 helicases in elf18-induced translation genetically, rh37 rh52, rh11 rh52, and rh11 rh37 double mutant lines were generated using a high efficiency CRISPR method (FIG. 13, panels a-c). Since the rh11 rh37 mutant exhibited a developmental defect, whereas the rh37 rh52 and the rh11 rh52 plants displayed near WT morphology (FIG. 13, panel d), it was elected to use rh37 rh52 and rh11 rh52 for targeted in planta SHAPE-MaP of the endogenous transcripts representing the TE-up and TE-nc groups. It was found that in the TE-up transcripts, the elf18-induced structural opening in the downstream regions of uAUGs was observed in WT, but diminished in the helicase double mutant (FIG. 4, panel e), supporting the involvement of RH11, RH37, and RH52 in elf18-induced unwinding of uAUG-ds. Moreover, we examined elf18-induced changes in the levels of four proteins with available antibodies in wild-type and helicase-mutant (rh37 rh52) plants and showed that increases in protein levels from those two transcripts containing translating uAUGs were dependent on the helicase activities (FIG. 13, panel e).

To examine the global effect of the helicase mutations on elf18-induced resistance against pathogens, bacterial infection was performed using Pseudomonas syringue pv. maculicola ES4326 (Psm ES4326) in WT, rh37 rh52 and rh11 rh52 mutants after pre-treating plants with elf18. As a negative control, the elf18 receptor mutant, eft was included. It was found that the helicase mutants had higher basal resistance to Psm ES4326 than the WT plants (FIG. 4, panel f), suggesting that they might affect transcripts other than those involved in pattern-triggered immunity. Nevertheless, the helicase mutants had significantly diminished sensitivity to elf18-induced resistance resulting in more overall bacterial growth (FIG. 4, panel f), which clearly demonstrates the indispensable roles of these helicases in translational regulation of pattern-triggered immunity Altogether, the results show that the elf18-inducible RNA helicase RH37 and its homologues RH11 and RH52 are involved in unwinding of uAUG-ds in TE-up transcripts and translation of downstream defense proteins against pathogen challenge.

Through this study, it was discovered that uAUG-ds are critical for dynamic start codon selection for translation initiation during plant pattern-triggered immunity. Without stress, translation of defense proteins is inhibited by uAUG-ds which slows the preinitiation complex scanning to engage the ribosome to initiation translation from uAUGs instead mAUGs. In response to stress, RH37-like helicases increase in expression and/or activity and become associated with the translation preinitiation complex to unwind uAUG-ds, thus promoting the bypass of uAUGs and translation of downstream defense proteins (FIG. 4, panel g).

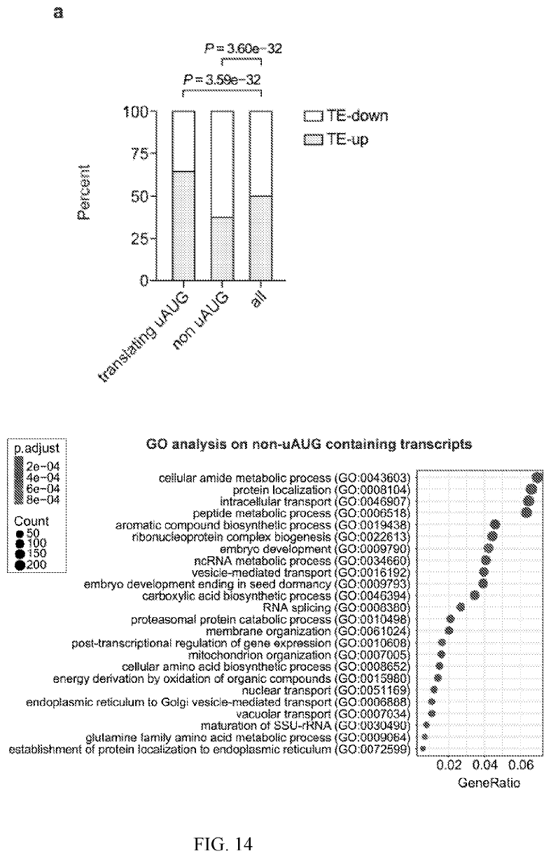

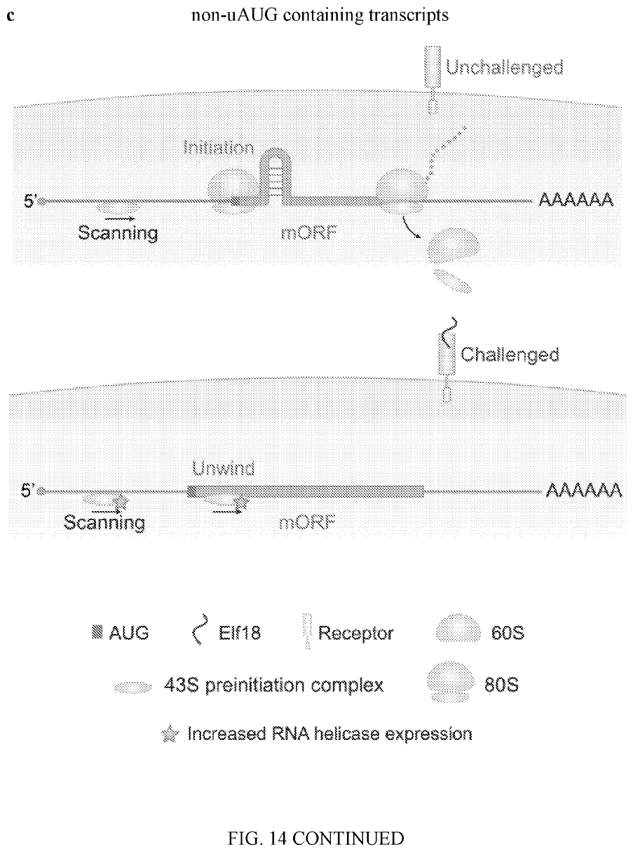

Even though this study was initiated to study uAUG-modulated translation in a plant immune response, the pervasive presence of the double-stranded RNA structures downstream of both mAUGs and translating uAUGs (FIG. 2, panels b, c), the unbiased deep learning results (FIG. 2, panels e-i and FIGS. 9 and 10) and the functional data obtained from studies in both plants and mammalian systems (FIG. 3 and FIG. 11) strongly support the fundamental importance of mAUG-ds and uAUG-ds in regulating translation in general. In contrast to the Kozak sequence context which is critical for start codon recognition under static conditions and is distinct between plants and animals, the uAUG-ds discovered in this study can be dynamically remodeled in response to stimuli to reprogram translation. In this study, a significant increase was not detected in SHAPE reactivities for mAUG-ds in the TE-up transcripts upon elf18 induction (FIG. 11, panel g). Notably, such dynamic regulation also occurs for transcripts that contain only mAUG-ds, which are enriched with transcripts in the TE-down category in response to elf18 treatment and found to encode growth-related proteins (FIG. 14, panels a, b). This finding indicates that immune-induced helicases can also unwind mAUG-ds and reduce translation from mAUG to inhibit the production of growth-related proteins (FIG. 14, panel c).

Discovery of the AUG-ds in this study was only possible through the integrated application of transcriptome-wide translational and structural analyses and deep learning algorithms because such structural features are unlikely to be detected through sequence homology. The strategy used here can be readily expanded to identify and characterize AUG-ds structures in other organisms, as AUG-ds are shown to regulate translation in different organisms (FIG. 3 and FIG. 11). Indeed, mAUG-ds could already be detected in the global mRNA structural datasets of yeast and C. elegans. Together with the fact that Ded1p/DDX3X/RH37 helicases are highly conserved from plants to humans (FIG. 12), it was hypothesized that the uAUG-ds/RNA helicase regulatory module is broadly present in eukaryotes. Moreover, the general features of mAUG-ds and uAUG-ds revealed in this study (FIG. 2, panels e-g) provide valuable information for rational design of protein synthesis for basic research as well as for applications in agriculture, in medicine and beyond. Using well-trained deep learning models in different organisms, potential uAUG-ds of functional genes can be identified to manipulate their translation. The success in engineering the inducible translational reporter which functions in plants as well as in human cells (FIG. 3, panel d, FIG. 4, panel d, and FIG. 11, panel b) provides confidence in the applicability of uAUG-ds as a new molecular switch for regulating gene expression.

### Example 5. Methods

Plant growth, elf 18 treatment, and transformation. Arabidopsis seedlings were grown on ½ Murashige and Skoog (MS) plates containing 0.8% agar and 1% sucrose or in soil, both at 22° C. under 12/12 h light/dark cycles with 55% relative humidity. All Arabidopsis plants used in the experiments were in the Col-0 background. N. benthamiana plants were grown under the same condition in soil as those for Arabidopsis for 4-5 weeks before experiments. For elf18 treatment, Arabidopsis seedlings were grown on plates for 7 days and transferred to liquid ½ MS solution and grown for one more day before being treated with 10 μM elf18 or water for 1 h. Transgenic plants were generated using the agrobacterium-mediated transformation method involving floral dipping.

Plasmid construction. The backbone (pTC090-32) for the dual-luciferase constructs used for expression in plants was generated in a previous study. The 5′ leader sequences of the TBF1, RH11 and RH37 transcripts were PCR-amplified from the Col-0 cDNA and of the TUB7 transcript was synthesized by IDT before being inserted into the backbone through ligation-based reactions (NEB) or using ClonExpress II One Step Cloning Kit (Vazyme). The site mutations and hairpin structures were introduced by primer-based PCR.

For expression in the mammalian cell line, dual-luciferase constructs were made using the backbone pSin obtained and modified from Addgen #16580. FLUC and RLUC reporters were inserted into pSIN to generate pSin-FLUC and pSin-RLUC, respectively. The 5′ leader sequence of the ATF4 transcript was PCR-amplified from the normal lung fibroblast cell line IMR90 cDNA. The 5′ leader sequence of the BRCA1 transcript was PCR-amplified from human breast cancer cell line MCF7 genomic DNA. All the 5′ leader sequences were cloned into pSIN-FLUC by Gibson Assembly (NEB). The site mutations and hairpin structures were introduced by primer-based PCR.

To generate the plasmids with dexamethasone-inducible expression of RNA helicases, the CDSs of RH11 and RH37 were PCR-amplified from the Col-0 cDNA and cloned into pBSDONR p1-p4, respectively. Each of these clones was then paired with the YFP tag, which was cloned in pBSDONR p4r-p2, to generate fusion constructs in the pBAV154 destination vector by multisite LR reaction (LR clonase II plus, ThermoFisher). The CRISPR knock-out lines were built through a highly efficient multiplex editing method. Briefly, to construct the shuttle vectors, four guide RNA sequences TAAACCGCCCGTGAACCACG (SEQ ID NO: 1), TAGACTCCCCGAACTCCACG (SEQ ID NO:2), TAGACTGTTCGTGAACCACG (SEQ ID NO:3), and TGGTCTTGACATTCCCCACG (SEQ ID NO:4) were loaded into the pDEG332, pDEG333, pDEG335 and pDEG337 modules, respectively. Then these guide RNA sequences were assembled into arrays in the recipient vector (pDGE666).

Ribo-seq and RNA-seq. Arabidopsis seedlings treated with elf18 or water as described above were collected, frozen in liquid nitrogen, and ground using the Genogrinder (SPEX SamplePrep). Polysome profiling was performed as described previously. Briefly, the ground tissue was homogenized in the polysome extraction buffer and subject to centrifuging to remove cell debris. The supernatant was then layered on top of a sucrose cushion and the ribosome pellet was collected after ultracentrifuge. The pellet was then washed with cold water and subject to RNase I (Ambion) digestion. The reaction was quenched by adding SUPERaseIn (Invitrogen). Ribosome-bound RNA was purified and subject to PNK treatment (NEB) and size selection through gel (Invitrogen) extraction. The recovered RNA was then subject to library preparation using NEBNext Multiplex Small RNA Library Prep Kit with slight modifications. Specifically, after the reverse transcription, rRNA depletion was performed. Briefly, the cDNA product was cleaned up with Oligo Clean & Concentrator Kit (Zymo) and then eluted with water. The eluted product was mixed with 0.4 nmol probes used in previous studies in the saline-sodium citrate (SSC) solution, and the mixture was subject to denaturation at 100° C. for 90 sec followed by a gradual decrease of temperature from 100° C. to 37° C. to allow annealing of the ribosomal DNA and the biotinylated oligos. The mixture was then incubated with 200 μg pre-washed Dynabeads MyOne Streptavidin C1 beads (Invitrogen) for 15 min at 37° C. with constant shaking. The tube was then placed on a magnetic rack for another 5 min and the flow-through was collected and cleaned up using Oligo Clean & Concentrator Kit (Zymo). This rDNA-depleted product was used as the template for PCR amplification and library preparation. Agilent 2100 Bioanalyzer was used for the sample quality control (FIG. 5, panel a). RNA from the same lysate was isolated and subject to library preparation using KAPA Stranded mRNA-Seq Kit (Roche). The six libraries for Ribo-seq (three mock and three elf18-induced) were pooled at equal amount of DNA and subject to the next-generation sequencing using Illumina NovaSeq (S2, full flow cell) with pair-end 50 bp. The six libraries for RNA-seq (three mock and three elf18-induced) were pooled at equal amount of DNA and subject to the next-generation sequencing using Illumina NovaSeq (S Prime, 1 lane) with pair-end 50 bp.

Ribo-seq and RNA-seq data processing. Ribo-seq read processing was performed following the steps illustrated in FIG. 6, panel a. Specifically, raw reads were trimmed using Trim Galore v0.6.6, a wrapper tool of Cutadapt and FastQC. The trimmed reads whose length is longer than or equal to 24 nt and shorter than or equal to 35 nt were kept and mapped to the rRNA and tRNA library from Arabidopsis TAIR 10 genome using Bowtie 2 v2.4.2. The unmapped reads were then assigned to Arabidopsis TAIR 10 genome using STAR v2.7.8a with -outFilterMismatchNmax 3-outFilterMultimapNmax 20-outSAMmultNmax 1-outMultimapperOrder Random. FastQC v0.11.9 and MultiQC v1.9 were applied for quality control during each step. Similarly, RNA-seq reads were trimmed and mapped using the same programs under default parameters.

To assess the data quality, the inventors first determined the read length distribution (FIG. 5, panel b) and the reads per kilobase of transcript per million mapped reads (RPKM) for all the transcripts in each replicate for the RNA-seq and Ribo-seq mapped reads using the featureCount program embedded in the Subread package v2.0.3, and plotted the Pearson correlations between every two replicates (FIG. 5, panels c, d). Then the inventors determined the P-site offset near start and stop codons for reads whose length ranging from 24 nt (24 mers) to 35 nt (35 mers) in Ribo-seq using Plastid v0.6.1 (FIG. 5, panel e). Next, the inventors determined the nucleotide periodicity 300 nt downstream of the start codons by calculating the power spectral density (FIG. 5, panel f). In addition, the inventors calculated RNA-seq and Ribo-seq reads distribution in the 5′ leader sequence, CDS, and 3′UTR of each transcript from mock- and elf18-treated samples (FIG. 5, panel g). A metaplot of the normalized distribution of Ribo-seq reads on the normalized transcript was calculated using the computational genomics analysis toolkit (CGAT)60 (FIG. 5, panel h). Translational efficiency changes were calculated using deltaTE. GO enrichment was performed online using the Gene Ontology resource and the results were visualized using enrichplot.

Identification of translating mAUGs and uAUGs. To identify transcripts with detectable translation initiation from mAUGs, the inventors analyzed 25554 detected transcripts that had RPKM of exon≥1 in all the six RNA-seq samples and RPKM of CDS≥1 in all the six Ribo-seq samples (FIG. 6, panel a). The inventors then calculated ribosome footprints spanning every mAUG for all the 25554 detected transcripts and normalized each count by total read count and transcript abundance. To set the background read count, the inventors took the top (Q3) quartile of the normalized read counts from regions 50 nt upstream of mAUGs of 5482 transcripts which have 5′ leader sequences≥100 nt without uAUGs (FIG. 6, panel b). Using the resulting background cut-off at 23.17, transcripts with normalized read counts at mAUG≥23.17 and with raw read counts at mAUG≥10 in all the six Ribo-seq samples were retained, and this yielded 13051 “expressed transcripts” with detectable translation initiation from mAUGs (FIG. 6, panel a).

To identify the uAUGs that can engage ribosome and facilitate translation initiation, the inventors performed similar calculation and normalization steps for ribosome footprints spanning every uAUG located in the 5′ leader sequences of all the 13051 expressed transcripts. uAUGs with normalized read counts≥23.17 and with raw read counts≥10 in all the three replicates in mock or/and in response to elf18 were selected and named as “translating uAUGs” (FIG. 6, panel a). A total of 5626 translating uAUGs were identified from the 13051 expressed transcripts. The rest 7968 uAUGs in the 13051 expressed transcripts are “non-translating uAUGs”.

In vivo SHAPE-MaP in plants and in mammalian cells. The SHAPE reagent, 2-methylnicotinic acid imidazolide (NAI), was synthesized as described in Spitale et al. For in vivo SHAPE-MaP in plants, Arabidopsis seedlings treated with elf18 or water or tobacco leaves transiently expressing the dual-luciferase reporters were collected and immediately immersed in the fresh NAI solution (100 mM NAI) or in the DMSO solution as previously described. To enhance the permeability of NAI, samples immersed in the solution were vacuum infiltrated and incubate at room temperature for 20 min. To quench the reaction, DTT (dithiothreitol, Roche) was added to the solution for a final concentration of 0.5 M and incubated for 2 min. The tissue was then washed with water for 3 times, frozen in liquid nitrogen, ground, and subject to total RNA isolation using Direct-zol RNA Miniprep Plus Kit (Zymo).

For in vivo SHAPE-MaP in the human HEK293FT cell line, cells were collected, washed once with cold 1×PBS after the removal of culture medium, and collected in a 1.5 mL tube. Cells were immediately resuspended in 500 μL fresh NAI solution (100 mM NAI) or in 500 μL DMSO solution and incubated at room temperature with gentle rotation for 5 min. The reaction was stopped by centrifuging the samples at 100,00 g in 4° C. for 1 min and removing the supernatant. The sample was immediately resuspended in Trizol (Invitrogen) and subjected to total RNA isolation using Direct-zol RNA Miniprep Plus Kit (Zymo).

The purified total RNA from plants or HEK293FT cells was subject to DNase treatment by adding 2 μL Turbo DNase (2 U/μL) and incubated in 37° C. for 30 min, followed by addition of another 2 μL Turbo DNase (2 U/μL) and incubated for another 30 min. RNA was then purified by RNA Clean & Concentrator Kit (Zymo). mRNA was enriched twice through poly(A) selection using Oligo d(T)25 Magnetic Beads (NEB), and subject to reverse transcription [mRNA in 2.5 μL nuclease-free water, 1 μL 10 mM dNTP (NEB), 1 μL Random Primer 9 (NEB), 2 μL 5×First-Strand Buffer (Invitrogen), 0.5 μL 0.2M DTT (Invitrogen), 0.5 μL TGIRT-III (InGex), 0.5 μL SUPERaseIn (Invitrogen), 2 μL 5M Betaine solution (Sigma-Aldrich)]. The cDNA product was cleaned up using Oligo Clean & Concentrator Kit (Zymo) and the library preparation was performed as described in Smola et al. under the randomer library preparation workflow. Agilent 2100 Bioanalyzer was used for the sample quality control. For the global SHAPE-MaP, libraries were pooled and subject to the next-generation sequencing using Illumina NovaSeq (S4, full flow cell) with pair-end 150 bp. For targeted SHAPE-MaP, gene-specific PCR primers were used for the library preparation as described in Smola et al. under the amplicon library preparation workflow.

In vitro SHAPE-MaP in plants. Arabidopsis seedlings treated in mock condition were collected, frozen in liquid nitrogen, ground, and subject to total RNA isolation using Direct-zol RNA Miniprep Plus Kit (Zymo). The purified RNA was subject to DNase treatment, clean-up, and poly(A) selection as mentioned above. To probe the in vitro RNA secondary structures, 500 ng purified mRNA was mixed with NAI (100 mM) or DMSO in a SHAPE reaction buffer (100 mM HEPES, 6 mM MgCl₂ and 100 mM NaCl) and incubated at room temperature for 5 min. The reaction was immediately quenched by purifying RNA using RNA Clean & Concentrator Kit (Zymo). The treated mRNA was then subject to reverse transcription, library preparation, and next-generation sequencing as described above.

SHAPE-MaP data processing. For global SHAPE-MaP data processing, raw reads were trimmed with Trim Galore v0.6.6 and the trimmed reads were mapped to rRNA and tRNA library from Arabidopsis TAIR 10 genome using Bowtie 2 v2.4.2, and the unmapped reads were aligned to Arabidopsis TAIR 10 transcriptome using Bowtie 2 v2.4.2. Mapped reads from all four replicates in each group were combined for the following analyses: (1) parse the mutations using shapemapper_mutation_parser; (2) count mutation events using shapemapper_mutation_counter; (3) summarize mutation events and calculate SHAPE reactivities using make_reactivity_profiles.py and normalize_profiles.py. Unless specified, only nucleotides with ≥1000 read coverage and with 0≤SHAPE reactivities≤6 were used for subsequent analyses to ensure accurate structural prediction. To examine the correlation between replicates, SHAPE reactivity for every transcript in each replicate was calculated individually, and the Pearson correlation coefficient for each transcript was determined in R v4.1.0 using the Hmisc package. For targeted SHAPE-MaP data processing, raw reads were processed using ShapeMapper 2. To ensure adequate read coverage and completeness, more than 100,000 reads/nucleotide were achieved for more than 90% of the targeted regions. Delta SHAPE reactivity was calculated by taking the log 2 fold change (elf18/mock) for each nucleotide, followed by data smoothing.

Training and validation of TISnet. To analyze the structure patterns in downstream regions of initiating AUG, a deep neural network was trained by adapting the PrismNet model. Downstream regions (101 nt) of mAUGs with high translational efficiency (“mAUGs”, n=2,857) were used as positive samples and downstream regions of AUGs randomly selected from CDSs or 3′UTRs (“internal AUGs”, n=7,143) were used as negative samples. Both positive and negative samples must have high SHAPE reactivity coverage (>25%). For each downstream region of AUG, the inventors predicted RNA secondary structures using RNAfold constrained by the SHAPE reactivity data, and trained TISnet to classify initiating and non-initiating AUGs by integrating sequence and secondary structure information.

More specifically, the positive samples were labelled as “1”, and negative samples were labelled as “0”. The sequence was then encoded by the one-hot encoding (A, C, G, U, 4-dimension), and encoded RNA secondary structures of each nucleotide to 0 or 1 (0 for nucleotides in double-stranded structures, 1 for nucleotides in single-stranded regions). The labels and encodings of samples were used as the input for the deep neural network. The positive and negative samples were then randomly split into a training set and a validation set by 4:1, and trained the network and validated the prediction performance of the network using the two sets, respectively.

Structure element identification. To find the sequence pattern of hairpin elements, the hairpin elements with long stems (>15 base pairs) were extracted from the downstream regions of predicted initiating AUGs. Then the inventors calculated the kmer (k=3) frequency of the loop sequences and the frequency of base pairs in each position (e.g., base pairs are counted starting from the loop) of the stem. Conserved structure elements were further identified by clustering hairpin elements into classes, based on the sequence similarity between each two hairpin elements. For two sequences, the inventors aligned them by the Needleman-Wunsch algorithm and defined sequence identity as:

$$Sequence identity = \frac{Number of aligned nucleotides}{Number of aligned and unaligned nucleotides}$$

The inventors divided each hairpin element into 5′ stem sequence (stem-1), loop sequence, and 3′ stem sequence (stem-2) (FIG. 10, panel c), and calculated the average of sequence identities of these three parts to represent the sequence similarity between two hairpin elements. The inventors calculated the sequence similarity between each two hairpin elements and clustered all hairpin elements in downstream regions of predicted initiating AUGs by the hierarchical clustering algorithm. For each hairpin element class, the inventors performed multiple alignment of the stem sequences and the loop sequences and calculated the frequency of nucleotides in each position to construct the position weight matrix (PWM) of the sequence motif. The secondary structures of downstream regions of AUGs were visualized by VARNA.

Dual-luciferase assay. Dual-luciferase assay for plant samples was conducted as described. Briefly, overnight culture of the Agrobacterium strain GV3101 transformed with the dual-luciferase construct was collected, resuspended in the infiltration buffer (10 mM MgCl₂, 10 mM MES and 200 M acetosyringone), adjusted to OD_{600 nm} at 0.2 and incubated at room temperature for an additional 2 h before infiltrating into N. benthamiana for transient expression. After 24 h of incubation, leaf discs were collected, ground in liquid nitrogen, and lysed with 1× passive lysis buffer (Promega). The lysate was centrifuged at 12,000 g for 3 min, and 10 μL supernatant was used for measuring FLUC and RLUC activities as previously described. For dex-induced expression experiment, the Agrobacterium strain with the dual-luciferase construct and the strain with the dex-inducible RNA helicase construct were co-infiltrated into N. benthamiana leaves and incubated for 20 h. Then the leaves were sprayed with 25 M dex solution in water and incubated for another 4 h before sample collection. Quantification on the Dex-induced proteins was performed using Western blotting assay. To detect YFP-tagged protein, the blot was probed with anti-GFP (Clontech, 632381, 1:5,000) primary antibodies and anti-mouse-HRP secondary antibodies (Abcam, Ab97040, 1:10,000). To detect HA-tagged proteins, the blot was probed with anti-HA HRP conjugated antibody (Cell Signaling Tech, 2999, 1:3,000).

Dual-luciferase assay in the human cell line was conducted according to manufacturer's instructions (Promega). Briefly, HEK293FT cells were seeded into 24-well plate and grown overnight to approximately 70% confluence at the time of transfection. 500 ng of pSin-RLUC and 500 ng of pSin-FLUC plasmids were co-transfected into HEK293FT cells using 2.5 μL Lipofectamine 2000 (Thermo Fisher Scientific, 11668019) for each well. After 24 h, cells were collected, washed once with cold 1×PBS after the removal of culture medium. 150 μL 1× passive lysis buffer (Promega) was used to extract the proteins according to standard procedures. 10 μL lysate was used for measuring FLUC and RLUC activities as previously described.

Elf18-induced resistance to Psm ES4326. The elf18-induced resistance experiment was performed as previously described. Briefly, Arabidopsis plants were grown in soil for 3-4 weeks and infiltrated with 1 M elf18 or Mock (water) one day before infection with Psm ES4326 (in 10 mM MgCl₂ solution at OD_{600 nm}=0.001) in the same leaf. Bacterial growth was measured two days after infection.

Statistics and reproducibility. Unless specified, statistical tests were performed using GraphPad Prism version 8.0 or in R v4.1.0. Statistical methods and number of experimental replications are indicated in figure legends. In the graphs, asterisks and lowercase letters indicate statistical significance reflecting the P values (*P<0.05, **P<0.01, ***P<0.001, ****P<0.0001, and ns, not significant). Unless specified, experiments were repeated at least three times with similar results.

Example 6. Various expression constructs were made to assess the translational impact of uAUG-ds-containing uORFs, considering factors such as the number of base pairings, folding energy, and different sequence contexts. Constructs for testing the uAUG-ds comprises a 35S promoter operably linked to a test uORF (containing a uAUG-ds), which was located 5′ of a FLUC heterologous ORF. FLUC expression is sensitive to the uORF. The test constructs further contained a RLUC reporter operably linked downstream of a second 25S promoter. The Dual-Luciferase reporter system was employed to assess the translational impact of the “Test” uORF sequence on the firefly luciferase (FLUC) reporter. The constitutively expressed Renilla luciferase (RLUC) served as a control in this analysis. The test uORFs are provided in SEQ ID NOs: 48-72. SEQ ID NOs: 48 corresponds to the uORF of the TBF1 gene (see FIG. 3, panel c). SEQ ID NO: 49 corresponds to a modified uORF of the TBF1 gene (see FIG. 3, panel c). SEQ ID NOs 50-57 correspond to artificial uAUG-ds, either alone or in combination with other known translational regulatory elements like the Kozak sequence, in the naïve TUB7 5′ leader sequence (see FIG. 3, panel d). SEQ ID NOs: 58-61 were used to test artificial uAUG-ds on the translation of the human ATF4 transcript (see FIG. 3, panel e). SEQ ID NOs: 62-65 were used to test the effect of uAUGs in the uAUG-ds-containing BRCA1 5′ leader sequence (see FIG. 3, panel f). SEQ ID NOs: 66-72 were used to test the effects of different strengths of dsRNA structures (underlined) on translation of the synthetic reporter (no uAUG) (see FIG. 11, panel c).

1. A messenger RNA (mRNA) comprising:
(a) an upstream open reading frame (uORF) comprising:
(i) an upstream start codon; and
(ii) a sequence that forms a stem loop structure operably linked to the upstream start codon, wherein the stem loop structure is about 1 to about 35 nucleotides downstream of the upstream start codon, and wherein the stem loop structure comprises a stem having about 12 to about 20 base pairs or a sequence that forms a secondary structure operably linked to the upstream start codon, wherein the secondary structure begins about 1 to about 35 nucleotides downstream of the upstream start codon and has a folding energy of about −19.9 kcal mol⁻¹ to about −34.1 kcal mol⁻¹ when calculated for nucleotides +4 to +104 relative to the upstream start codon; and
(b) a heterologous open reading frame (ORF) encoding a polypeptide,
wherein the uORF is 5′ of the heterologous ORF and is operably linked to the heterologous ORF, and
wherein the uORF regulates translation of the heterologous ORF.

2. The mRNA of claim 1, wherein the upstream start codon comprises an upstream AUG (uAUG).

3. The mRNA of claim 1 or 2, wherein the stem loop structure starts at about position +9 to about position +26 nucleotides, about position +13 to about position +17, or about position +15.

4. The mRNA of any one of claims 1-3, wherein the uORF comprises the sequence that forms the stem loop structure, wherein the stem further has a folding energy of about −19.9 kcal mol⁻¹ to about −34.1 kcal mol⁻¹ or −26.8±5 kcal mol⁻¹.

5. The mRNA of any one of claims 1-4, wherein the percentage GC content of the stem of the stem loop structure is about 46% to about 61%, at least 50%, or is greater than the percentage GC content of the loop of the stem loop structure, optionally wherein the percentage GC content of the loop of the stem loop structure is about 28% to about 50%, or less than 50%, optionally wherein the stem loop structure comprises SEQ ID NO: 73.

6. The mRNA of any one of claims 1-5, wherein the heterologous ORF encodes a protein selected from the group consisting of:
(i) a polypeptide that is a transcription factor;
(ii) a reporter polypeptide;
(iii) a polypeptide that confers resistance to drugs or agrichemicals;
(iv) a polypeptide involved in resistance of plants to viral pathogens, bacterial pathogens, fungal pathogens, oomycete pathogens, phytoplasmas, or nematodes; and
(v) a polypeptide involved in the growth or development of plants.

7. The mRNA of any one of claims 1-6, wherein the heterologous ORF encodes a gene selected from the group consisting of: MLO, EDR1, Pi21, OsSWEET11, OsSWEET13, OsSWEET14, eIF4E, DMR6 (SlDMR6-1), Sr35, Sr50, Sr33, Pik1 and Pik2, RGA5 and RGA4, RRS1 and RPS4, RBS1, CsLOB1, PBS1, Xa27, MAPK3K StVIK1, COI1, IPA1, OsHEN1, SNC1, NPR1, CDP-DAG, RBL1. BBS1, HPL3, CSLF6, LIL1, RLIN1, SPL3 (OsEDR1 ACDR1), LMS, OsLOL1, MLO, OsSL, LRD6-6, SPL5, SPL7, SPL11, SPL18, SPL28, ACLA2, ABC1, SPL33, OsSPL35, SPL40, WSP1, XB15, OsSSI2, GF14e, NOE1, EBR1, OsCUL3a, NLS1, OsRLR1, SNC1, SSI4, SLH1, CHS3-2D, CHS3-1, CHS2, UNI-1D, BAK1, BKK1, BIR1, SNC4-1D, CERK1-4, SNC2-1D, RIN4, CPR1, SRFR1, CPN1/BON1, MKP1, LSD1, ACD11, CPR5, MEKK1, MPK4, MKK1, MKK2, ACD6, BDA1-17, DND1, DND2/HLM1, CPR22, NPR3 NPR4, SR1/CAMTA3, PUB13, CPR6-1, SSI2, SYP121, SYP122, CAD1, NSL1, and orthologs thereof.

8. The mRNA of any one of claims 1-7, wherein the translation of the heterologous ORF is inducible.

9. The mRNA of claim 8, wherein translation of the heterologous ORF is induced by a stress response, an immune response, a stress-induced helicase, an immune response-induced helicase, or an antisense oligonucleotide that specifically hybridizes to a sequence in the stem loop structure.

10. The mRNA of claim 9, wherein the stress-induced helicase or the immune response-induced helicase comprises a RH37 helicase or an ortholog thereof, a RH11 helicase or an ortholog thereof, a RH52 helicase or an ortholog thereof, a Ded1p helicase or an ortholog thereof, or a DDX3X helicase or an ortholog thereof.

11. A DNA molecule comprising a sequence encoding the mRNA of any one of claims 1-10.

12. The DNA molecule of claim 11, wherein the DNA molecule further comprises a promoter sequence operably linked to the sequence encoding the mRNA.

13. The DNA molecule of claim 12, wherein the promoter is selected from the group consisting of:
(a) a plant promoter;
(b) a plant virus promoter;
(c) a promoter from a non-viral plant pathogen;
(d) a mammalian cell promoter; and
(e) a mammalian virus promoter.

14. The DNA molecule of any one of claims 11-13, wherein the DNA molecule further encodes a helicase, optionally wherein the helicase comprises a RH37 helicase or an ortholog thereof, a RH11 helicase or an ortholog thereof, a RH52 helicase or an ortholog thereof, a Ded1p helicase or an ortholog thereof, or a DDX3X helicase or an ortholog thereof.

15. A vector comprising the mRNA of any one of claims 1-10 or the DNA molecule of any one of claims 11-14.

16. The vector of claim 15, wherein the vector comprises a viral vector, a transposon, a plasmid, or a CRISPR system.

17. A modified cell comprising the mRNA of any one of claims 1-10 or the DNA molecule of any one of claims 11-14, optionally wherein the cell is a mammalian cell or a plant cell.

18. A plant propagation material comprising one or more modified cells of claim 17.

19. A plant comprising one or more modified cells of claim 17, optionally wherein the plant is a transgenic plant.

20. A DNA molecule encoding a RNA comprising:
(a) an upstream open reading frame (uORF) comprising:
(i) an upstream start codon; and
(ii) a sequence that forms a stem loop structure operably linked to the upstream start codon, wherein the stem loop structure is about 1 to about 35 nucleotides downstream of the upstream start codon, and wherein the stem loop structure comprises a stem having about 12 to about 20 base pairs or a sequence that forms a secondary structure operably linked to the upstream start codon, wherein the secondary structure begins about 1 to about 35 nucleotides downstream of the upstream start codon and has a folding energy of about −19.9 kcal mol⁻¹ to about −34.1 kcal mol⁻¹ when calculated for nucleotides +4 to +104 relative to the upstream start codon; and
(b) a heterologous sequence comprising a synthetic polylinker, a ligation independent cloning sequence, a sequence recognized by one or more restriction enzymes, or a heterologous open reading frame (hORF) encoding a polypeptide,
wherein the uORF is 5′ of the heterologous sequence and is operably linked to the heterologous sequence.

21. A method for generating a cell comprising an inducibly expressed polypeptide, the method comprising:
(a) introducing the mRNA of any one of claims 1-11 into the cell;
(b) introducing the DNA molecule of any one of claims 11-14 and 20 into the cell; and/or
(c) introducing the vector of any one of claims 15-16 into the cell.

22. A method for generating a cell in which a polypeptide can be inducibly expressed, the method comprising modifying an endogenous gene encoding the polypeptide in the cell to produce a modified gene, wherein the modified gene encodes an mRNA comprising:
(a) a heterologous upstream open reading frame (uORF) comprising:
(i) an upstream start codon; and
(ii) a sequence that forms a stem loop structure operably linked to the upstream start codon, wherein the stem loop structure is about 1 to about 35 nucleotides downstream of the upstream start codon, and wherein the stem loop structure comprises a stem having about 12 to about 20 base pairs or a sequence that forms a secondary structure operably linked to the upstream start codon, wherein the secondary structure begins about 1 to about 35 nucleotides downstream of the upstream start codon and has a folding energy of about −19.9 kcal mol⁻¹ to about −34.1 kcal mol⁻¹ when calculated for nucleotides +4 to +104 relative to the upstream start codon; and
(b) an open reading frame (ORF) encoding the polypeptide,
wherein the heterologous uORF is 5′ of the ORF and is operably linked to the ORF.

23. The method of claim 22, wherein translation of the ORF is induced by a stress response, an immune response, a stress-induced helicase, an immune response-induced helicase, or an antisense oligonucleotide that specifically hybridizes to a sequence in the stem loop structure.

24. The mRNA of claim 23, wherein the stress-induced helicase or the immune response-induced helicase comprises a RH37 helicase or an ortholog thereof, a RH11 helicase or an ortholog thereof, a RH52 helicase or an ortholog thereof, a Ded1p helicase or an ortholog thereof, or a DDX3X helicase or an ortholog thereof.

25. The method of any one of claims 22-24, wherein the method further comprises expressing a heterologous helicase in the cell, optionally wherein the helicase comprises a RH37 helicase or an ortholog thereof, a RH11 helicase or an ortholog thereof, a RH52 helicase or an ortholog thereof, a Ded1p helicase or an ortholog thereof, or a DDX3X helicase or an ortholog thereof.

26. The method of any one of claims 22-24, wherein the method further comprises contacting the cell with an antisense oligonucleotide that specifically hybridizes to a sequence in the stem loop structure.

27. A messenger RNA (mRNA) comprising:
(i) a start codon; and
(ii) a sequence that forms a stem loop structure operably linked to the start codon, wherein the stem loop structure is about 1 to about 35 nucleotides downstream of the start codon, and wherein the stem loop structure comprises a stem having about 12 to about 20 base pairs or a sequence that forms a secondary structure operably linked to the start codon, wherein the secondary structure begins about 1 to about 35 nucleotides downstream of the start codon and has a folding energy of about −19.9 kcal mol⁻¹ to about −34.1 kcal mol⁻¹ when calculated for nucleotides +4 to +104 relative to the start codon.

28. The mRNA of claim 27, wherein the stem loop structure starts at about position +9 to about position +26, about position +13 to about position +17, or about position +15.

29. The mRNA of claim 27 or 28, wherein the percentage GC content of the stem of the stem loop structure is about 46% to about 61%, is at least 50%, or is less than the percentage GC content of the loop of the stem loop structure, optionally wherein the percentage GC content of the loop of the stem loop structure is about 28% to about 50%, or less than 50%.

30. The mRNA of any one of claims 27-29, wherein the polypeptide comprises a therapeutic protein.

31. A DNA molecule comprising a sequence encoding the mRNA of any one of claims 27-30.

32. The DNA molecule of claim 31, wherein the DNA molecule further comprises a promoter sequence operably linked to the sequence encoding the mRNA, optionally wherein the promoter is selected from the group consisting of: a plant promoter; a plant virus promoter; a promoter from a non-viral plant pathogen; a mammalian cell promoter; and a mammalian virus promoter.

33. A vector comprising the mRNA of any one of claims 27-30 or the DNA molecule of any one of claims 31-32, optionally wherein the vector comprises a viral vector, a transposon, a plasmid, or a CRISPR system.

34. A modified cell comprising the mRNA of any one of claims 27-30 or the DNA molecule of any one of claims 31-32, optionally wherein the cell is a mammalian cell, or a plant cell.

35. A method for increasing translation of an ORF in a cell, the method comprising: modifying a nucleic acid encoding the ORF to contain
(a) a stem loop structure, wherein the stem loop structure is operably linked to the start codon of the ORF, is about 1 to about 35 nucleotides downstream of the start codon, and comprises a stem having about 12 to about 20 base pairs; or
(b) a sequence that forms a secondary structure operably linked to the start codon or the ORF, wherein the secondary structure begins about 1 to about 35 nucleotides downstream of the start codon and has a folding energy of about −19.9 kcal mol⁻¹ to about −34.1 kcal mol⁻¹ when calculated for nucleotides +4 to +104 relative to the start codon.

36. The method of claim 35, wherein modifying the nucleic acid encoding the ORF comprises substituting one or more codons downstream of the ORF thereby forming the stem loop structure, optionally wherein substituting the one or more codons does not change the encoded amino acid sequence or results in one or more conservative amino acids changes to the coding sequence.

37. A method for increasing translation of a gene having an upstream open reading frame comprising an upstream start codon and a sequence that forms a stem loop structure operably linked to the upstream start codon, wherein the stem loop structure is about 1 to about 35 nucleotides downstream of the upstream start codon, the method comprising contacting a cell containing the gene with an antisense oligonucleotide that specifically hybridizes to a sequence in the stem loop structure.


SEQUENCE LISTING (93 sequences)

SEQ ID NO: 1 - DNA, 20 bp, synthetic construct
  source 1..20: mol_type = other DNA
taaaccgcccgtgaaccacg

SEQ ID NO: 2 - DNA, 20 bp, synthetic construct
  source 1..20: mol_type = other DNA
tagactccccgaactccacg

SEQ ID NO: 3 - DNA, 20 bp, synthetic construct
  source 1..20: mol_type = other DNA
tagactgttcgtgaaccacg

SEQ ID NO: 4 - DNA, 20 bp, synthetic construct
  source 1..20: mol_type = other DNA
tggtcttgacattccccacg

SEQ ID NO: 5 - RNA, 101 nt, synthetic construct
  source 1..101: mol_type = other RNA
gaagaaaccaaacgaaactccgatcttctccgttctcgtgttttcctctctggcttttattgctgggattgggaatttctcaccgctctcttgctttttag

SEQ ID NO: 6 - RNA, 101 nt, synthetic construct
  source 1..101: mol_type = other RNA
gctctgtttagagatattgttcttcttgggtttctcttctgcttgagcttagtagctactgtgacttcagaggagggagcaacgttgctggagattaagaa

SEQ ID NO: 7 - RNA, 65 nt, synthetic construct
  source 1..65: mol_type = other RNA
ggggacactatgtctttaaaaacgtcggctggtcatgaggtcaggagttccagaccagcctgacc

SEQ ID NO: 8 - RNA, 487 nt, synthetic construct
  source 1..487: mol_type = other RNA
ttgtctcaaagattaagccatgcatgtgtaagtatgaacgaattcagactgtgaaactgcgaatggctcattaaatcagttatagtttgtttgatggtaactactactcggataaccgtagtaattctagagctaatacgtgcaacaaaccccgacttatggaagggacgcatttattagataaaaggtcgacgcgggctctgcccgttgctctgatgattcatgataactcgacggatcgcatggcctctgtgctggcgacgcatcattcaaatttctgccctatcaactttcgatggtaggatagtggcctaccatggtggtaacgggtgacggagaattagggttcgattccggagagggagcctgagaaacggctaccacatccaaggaaggcagcaggcgcgcaaattacccaatcctgacacggggaggtagtgacaataaataacaataccgggctctttcgagtctggtaattggaatg

SEQ ID NO: 9 - DNA, 16 bp, synthetic construct
  source 1..16: mol_type = other DNA
cggtgaagtacttagg

SEQ ID NO: 10 - DNA, 101 bp, synthetic construct
  source 1..101: mol_type = other DNA
gcgattttggattctgctggcgttactacggtgacggagaacggtggcggagagttcgtcgatcttgataggcttcgtcgacggaaatcgagatcggattc

SEQ ID NO: 11 - DNA, 101 bp, synthetic construct
  source 1..101: mol_type = other DNA
gctgggatttgttgcggtgttgttggagagactgaaccggcggcaccggttgattcaacttctcgagcttctcttagacggaggttagatctacttccgtc

SEQ ID NO: 12 - DNA, 101 bp, synthetic construct
  source 1..101: mol_type = other DNA
acaaccatagctgcagctggcctcaacgtcgcgactccacgagtggtcgttcgacctgtggctcgtgtattaggtccggtccggttgaattacccgtggaa

SEQ ID NO: 13 - DNA, 101 bp, synthetic construct
  source 1..101: mol_type = other DNA
ggtggagtgaagtggagcttggaaagttttacattaacaatggaggttgtggtgatgatggtagtgatgagattgagattagtataatggagactcagaat

SEQ ID NO: 14 - DNA, 101 bp, synthetic construct
  source 1..101: mol_type = other DNA
gaaaagccaagaaacttggactctgttgataagtctaaacctttagagctcacagaaattgttgatccgactcagtgccgacaagtgacaatgccagatag

SEQ ID NO: 15 - DNA, 101 bp, synthetic construct
  source 1..101: mol_type = other DNA
ggaatggattggtggcttatagagctgtgaaatgggcttctccgactgagtttgttactggaggatatggttttggtttgcaattgtgggatcagaggaag

SEQ ID NO: 16 - DNA, 101 bp, synthetic construct
  source 1..101: mol_type = other DNA
aagaacaagaccaatttaggtctctcagtcttcttcttctttatatgtctcgtttccgtgacatcagatctagaagctgatcgacgagctctaatcgcact

SEQ ID NO: 17 - DNA, 101 bp, synthetic construct
  source 1..101: mol_type = other DNA
gatcatcatcttcaaccataacaacacgaaacccttcttctccaaaatccccaaaaccttgaaacaaagtctcaaaatttcacttcgtggatccgaatgtc

SEQ ID NO: 18 - RNA, 427 nt, synthetic construct
  source 1..427: mol_type = other RNA
tttgtgatctataaatcttcctactacgttatatagagacccttcgacataacacttaactcgtttatatatttgttttacttgttttgcacatacacacaaaaataaaaaagactttatatttatttactttttaatcacacggattagctccggcgaagtatggtcgtcgtcttcatcttcttcctccatcatcagatttttccttaaatggaagaaaccaaacgaaactccgatcttctccgttctcgtgttttcctctctggcttttattgctgggattgggaatttctcaccgctctcttgctttttagttgctgattctttttccttcgactttctatttccaatctttcttcttctctttgtgtattagattatttttagttttatttttctgtggtaaaataaaaaaagttcgccggag

SEQ ID NO: 19 - RNA, 427 nt, synthetic construct
  source 1..427: mol_type = other RNA
tttgtgatctataaatcttcctactacgttatatagagacccttcgacataacacttaactcgtttatatatttgttttacttgttttgcacatacacacaaaaataaaaaagactttatatttatttactttttaatcacacggattagctccggcgaagtatggtcgtcgtcttcatcttcttcctccatcatcagatttttccttaaatggaagaaaccaaagtaaactttgatgttattcattctcgtgttttcctctctggcttttattgctgggattgggaatttctcaccgctctcttgctttttagttgctgattctttttccttcgactttctatttccaatctttcttcttctctttgtgtattagattatttttagttttatttttctgtggtaaaataaaaaaagttcgccggag

SEQ ID NO: 20 - protein, 515 aa, Homo sapiens
MSHVAVENALGLDQQFAGLDLNSSDNQSGGSTASKGRYIPPHLRNREATKGFYDKDSSGWSSSKDKDAYSSFGSRSDSRGKSSFFSDRGSGSRGRFDDRGRSDYDGIGSRGDRSGFGKFERGGNSRWCDKSDEDDWSKPLPPSERLEQELFSGGNTGINFEKYDDIPVEATGNNCPPHIESFSDVEMGEIIMGNIELTRYTRPTPVQKHAIPIIKEKRDLMACAQTGSGKTAAFLLPILSQIYSDGPGEALRAMKENGRYGRRKQYPISLVLAPTRELAVQIYEEARKFSYRSRVRPCVVYGGADIGQQIRDLERGCHLLVATPGRLVDMMERGKIGLDFCKYLVLDEADRMLDMGFEPQIRRIVEQDTMPPKGVRHTMMFSATFPKEIQMLARDFLDEYIFLAVGRVGSTSENITQKVVWVEESDKRSFLLDLLNATGKDSLTLVFVETKKGADSLEDFLYHEGYACTSIHGDRSQRDREEALHQFRSGKSGGFYNSDGYGGNYNSQGVDWWGN

SEQ ID NO: 21 - protein, 471 aa, Saccharomyces cerevisiae
MAELSEQVQNLSINDNNENGYVPPHLRGKPRSARNNSSNYNNNNGGYNGGRGGGSFFSNNRRGGYGNGGFFGGNNGGSRSNGRSGGRWIDGKHVPAPRNEKAEIALFGVPEDPNFQSSGINFDNYDDIPVDASGKDVPEPITEFTSPPLDGLLLENIKLARFTKPTPVQKYSVPIVANGRDLMACAQTGSGKTGGFLFPVLSESFKTGPSPQPESQGSFYQRKAYPTAVIMAPTRELATQIFDEAKKFTYRSWVKACVVYGGSPIGNQLREIERGCDLLVATPGRLNDLLERGKISLANVKYLVLDEADRMLDMGFEPQIRHIVEDCDMTPVGERQTLMFSATFPADIQHLARDFLSDYIFLSVGRVGSTSENITQKVLYVENQDKKSALLDLLSASTDGLTLIFVETKRMADQLTDFLIMQNFRATAIHGDRTQSERERALAAFRSGAAGSDSKSSGWGNSGGSNNSSWW

SEQ ID NO: 22 - protein, 502 aa, Arabidopsis thaliana
MSASWADVADSENTGSGSSNQNSHPSRPAYVPPHLRNRPAASEPVAPLPANDRVGYGGPPSGSRWAPGGSGVGVGGGGGYRADAGRPGSGSGYGGRGGGGWNNRSGGWDRREREVNPFENDDSEPEPAFTEQDNTVINFDAYEDIPIETSGDNVPPPVNTFAEIDLGEALNLNIRRCKYVKPTPVQRHAIPILLEGRDLMACAQTGSGKTAAFCFPIISGIMKDQHVQRPRGSRTVYPLAVILSPTRELASQIHDEAKKFSYQTGVKVVVAYGGTPINQQLRELERGVDILVATPGRLNDLLERARVSMQMIRFLALDEADRMLDMGFEPQIRKIVEQMDMPPRGVRQTLLFSATFPREIQRLAADFLANYIFLAVGRVGSSTDLIVQRVEFVLDSDKRSHLMDLLHAQRENGIQGKQALTLVFVETKRGADSLENWLCINGFPATSIHGDRTQQEREVALKAFKSGRTGGGGGYGGGGGYGGGYGGASSGGYGGEPPSAWD

SEQ ID NO: 23 - protein, 512 aa, Arabidopsis thaliana
MSSNSWADVSESERAPSGGGWGYSRPSRTNYVPPHLRSRTPSSEFVAPSPGNNDRGGYGGANSGYGGRGQGYGGRGSGYGGRGGPVGGWNARSGGWDRRDTETNPFGNDGNADPAVNEQENTVINFEAYEDIPIETSGDNVPPPVNTFAEIDLGEALNLNIQRCKYVKPTPVQRNAIPILAAGRDLMACAQTGSGKTAAFCFPIISGIMKDQHIERPRGVRGVYPLAVILSPTRELACQIHDEARKFSYQTGVKVVVAYGGTPVNQQIRELERGVDILVATPGRLNDLLERGRVSLQMVRFLALDEADRMLDMGFEPQIRKIVQQMDMPPPGVRQTMLFSATFPREIQRLASDFLSNYIFLAVGRVGSSTDLIVQRVEFVHDSDKRSHLMDLLHAQRENGNQGKQALTLVFVETKKGADSLENWLCINGFPATTIHGDRSQQEREVALRSFKTGRTGAMPGGYGPVPGGGYGNVPGGGYAPYGRGGGAYYGPGGYGTVPNQGYGPGVASAWD

SEQ ID NO: 24 - protein, 481 aa, Arabidopsis thaliana
MSASWADVADSEKAVSQSKPPYVPPHLRNRPSEPVAAPLPQNDHAGYGGQPAGSRWAPPSSGGGGASGGGYRNDGGRTGYGYGAGGGGGGGGGWNNRSGGWDRREREVNPFGDDAELEPVFTEQENTGINFDAYEDIPVETSGGDVPPPVNTFADIDLGDALNLNIRRCKYVRPTPVQRHAIPILLAERDLMACAQTGSGKTAAFCFPIISGIMKDQHVERPRGSRAVYPFAVILSPTRELACQIHDEAKKFSYQTGVKVVVAYGGTPIHQQLRELERGCDILVATPGRLNDLLERARVSMQMIRFLALDEADRMLDMGFEPQIRKIVEQMDMPPRGVRQTMLFSATFPSQIQRLAADFMSNYIFLAVGRVGSSTDLITQRVEFVQESDKRSHLMDLLHAQRETQDKQSLTLVFVETKRGADTLENWLCMNEFPATSIHGDRTQQEREVALRSFKTGRTGGGGYGGAPSGGYGAGVTSAWD

SEQ ID NO: 25 - protein, 484 aa, Medicago truncatula
MRSSWADLAANSAAENAPTPPPSAAAAVANNAANTRPVYVPPHLRNRGPSAPAPAAPAFDNSGSRWAPPPRNDYRGGGGGRGGGGYGNRGGGGWDRREANPFADQDDSEEPVTQEEQENTGINFDAYEDIPVETSGGNVPPPVNTFAEIDLGEALNQNIRRCKYVKPTPVQRHAIPISLGGRDLMACAQTGSGKTAAFCFPIISGIMTGQPAQRPPRGVRTVCPLALVLSPTRELSMQIHEEARKFSYQTGVRVVVAYGGAPINQQLRELERGVDILVATPGRLVDLLERARVSLSMIRYLALDEADRMLDMGFEPQIRKIVEQMDMPPAGVRQTMLFSATFPKEIQRLASDFLSNYIFLAVGRVGSSTDLIDQRVEYVQESDKRSHLMDLLHAQRANGVQGKQALTLVFVETKKGADALEHWLCLNNFPATTIHGDRSQQEREAALRSFKSGNTGASGGYGGGGYGGGYAGNSAGPGVTSAWD

SEQ ID NO: 26 - protein, 482 aa, Medicago truncatula
MSATMRTATWADSADNAANASSTARPVKPAYVPPHLRNRSMAAPAEPPSLVANDRGLGNNWGSSSNFKQDFGAGRQVGGGYGGGGGGFGGFNRGGAGNGRERVGGGGGRREANPFENDVDEPFTEQENTGINFDAYDDIPVETSGENVPPPVNTFAEIDLGDALNQNIKRCKYVKPTPVQRYAIPISLAGRDLMACAQTGSGKTAAFCFPIISGILTEQYAQRPRVARTAYPLALILSPTRELSCQIHDEAKKFSYQTGVKVVVAYGGAPITQQLRELERGVDILVATPGRLVDLLERARVSLQMIRYLALDEADRMLDMGFEPQIRKIVEQMDMPPPGMRQTLLFSATFPKEIQRLASDFLASYIFLAVGRVGSSTDLIAQRVEYVLESDKRSHLMDLLHAQRENDVNGKQGLTLVFVETKKGADALEHCLCVNGFPATCIHGDRTQQEREQALRSFKTGNTGVPASYGGGYGQGVTSAWD

SEQ ID NO: 27 - protein, 496 aa, Medicago truncatula
MAAAWSDSTTQKQNRPSQQLYVPQFRSNPNNNNIHPSPYYNNNRTHFPSNDSPHYNTFNRPTPRPTRGRGGYGFGHSPAPPRHYSRPEQQQPRYYDPSQNRSPFETNGEKVENGVVANFEAYESIPVEATGENVPPPVNLFAETELHEDLKNNIERCKYVKPTPVQRYAIPIAVAGRDLMACAQTGSGKTAAFCFPIISGIMKERLASGLLPRGGGVDSDVAFPTALILSPTRELSCQIHAEACKFAHQTGVKIAVVYGGAPIGQQLRILERGVDILVATPGRLVDLIERERVSLKKIKYLALDEADRMLDMGFEHQIRNIVQQMHMPPPGDRQTLLFSATFPDNIQKLASDFLSNYVFLAVGRVGSSTELIVQKIESVQDMEKRTRLVDLLRRNVVNGKLALTLVFVETKKGADALENWLCRINFPAIAIHGDKVQMERERALRSFKRGLTDCSFDNTNTVVAGGFNYTDAGLLEGSDGTNGPCGYASVVPTGWD

SEQ ID NO: 28 - protein, 479 aa, Solanum lycopersicum
MRNSWADSVENVATDNGGPSGASGASASTRRSSYVPPHLRNRPMTAEPLAPSHGGPPSGNDRSGFNGPTSGPRWGGSRPDYGRQGGYGGGGRGGGWGGRGGGWGREREVNPFGNDADVDLEQSFDQENTGINFDAYEDIPVETSGENVPPPVNTFAEIDLGEAVNLNIRRCKYVKPTPVQRYAIPISLAGRDLMACAQTGSGKTAAFCFPIISGIMRGHFPQRPHGSRTVFPLALILSPTRELSMQIHEEAKKFSYQTGVRVVVAYGGAPINQQLRELERGVDILVATPGRLVDLLERAKVSLQMIRYLALDEADRMLDMGFEPQIRRIVQQMDMPPPGERQTMLFSATFPKEIQRLASDFLANYIFLAVGRVGSSTDLIVQRVEFVHDVDKRSHLMDLLHAQMANGVHGKHSLTLVFVETKKGADALEHWLCINGFPATAIHGDRTQQEREQALRTFKRGDTGNYSGGYGPGVTSAWD

SEQ ID NO: 29 - protein, 472 aa, Solanum lycopersicum
MSMSWADSVSAAENPAGAPSKSAYVPPHLRNRPSTDPPAQASYAGAAASLGNDHSGYGVPNMVGSRWSGQRNEYQSGYAGGGGRGGGWNGRSGGWDRGRVREVNPFTNDDSEPVTEQESSAINFDAYEDIPVETSGDNVPPPVNTFAEIDLGEAVNENIRRCKYVKPTPVQRHAIPISLAGRDLMACAQTGSGKTAAFCFPIISGIMRGQFPRPPRPRMAFPLALILSPTRELSCQIHDEAKKFSYQTGVRVVVAYGGAPINQQLRELERGVHILVATPGRLVDLLERARVSLQMIRYLALDEADRMLDMGFEPQIRKIVQQMDMPPPGVRQTMLFSATFPKEIQRLASDFLSSYIFLAVGRVGSSTDLIVQRVEYVQETDKRSHLMDLLHAQKENGVHGKQALTLVFVETKKGADALEHWLCMNGFPAAAIHGDRTQQEREHALRSFKTGYTASGGYNASYGTAGVTSAWD

SEQ ID NO: 30 - protein, 506 aa, Oryza sativa
MRSSWADSAANAEESAPAAAANHGNSRLPRSSYVPPHLRGQAAPAAPAQAGALPSAAAAAQPSVGQPGVVGGPRWAGIVNGGGGGGGGSVGGSRQGFGAGGRGGGGGGGGGAWNSRPGGWDRRDREPDPFANSEAAEVDFEGENTGINFEAYEDIPVETSGHDVPPPANTFAEIDLGDALNENIRRCKYVKPTPVQRYAIPISIAGRDLMACAQTGSGKTAAFCFPIISGIMRSRPPPRSRGSRTAYPLALILSPTRELSVQIHEEARKFAYQTGVKVVVAYGGAPITQQLRELERGVEILVATPGRLMDLLERARVSLQMIKYLALDEADRMLDMGFEPQIRKIVEQMDMPPRGERQTMLFSATFPKEIQRMASDFLADYIFLAVGRVGSSTDLIVQRVEFVLDADKRSYLMDLLHAQRANGTHGKQALTLVFVETKRGADALENWLYNNGFPATSIHGDRTQQEREYALRSFKSGATGGGGGGGGYGGGSSYGGGGQGFSSAWD

SEQ ID NO: 31 - protein, 505 aa, Oryza sativa
MRSSWADSVANAEESAPATGAAPTPVANHQNSRPTRSAYVPPHLRGQAPTTTAAPAPAPGPAAVQPSASVQPSGYAAIVGGSRWAGPASGDGTGAVGGPRQSVGGRGGGGGGGGGWNSRPGWDRRDREPNPFANSEAEEATEVDFDTANTGINFDAYEDIPVETSGHDVPPPVNTFAEIDLGDALNENIRRCKYVKPTPVQRYAIPISIAGRDLMACAQTGSGKTAAFCFPIISGIMSSRPPQRPRGSRTAYPLALILSPTRELSVQIHEEARKFAYQTGVRVVVAYGGAPIHQQLRELERGVEILVATPGRLMDLLERARVSLQMVKYLALDEADRMLDMGFEPQIRKIVEQMDMPPRGVRQTMLFSATFPKEIQRMASDFLADYIFLAVGRVGSSTDLIAQRVEFVLEADKRSYLMDLLHAQKANGTHGKQALTLVFVETKRGADALENWLYTNGFPATSIHGDRTQQEREYALRSFKSGATGGGGGYGGGYGGGQGSTSSWD

SEQ ID NO: 32 - protein, 489 aa, Oryza sativa
MATPSRTSWADVADADPAPAPAPAANGPARPDRSSYVPPHLRNRGASSGGGAAAPPPSSSSSSAPPPRAAPGLLAPRPAAAGMGRMGGGGGGGGFGGPRRWDREPNPFGNDGDAAAGAGDEPEVFDAHQNTGINFDAYEDIPVETSGREVPPPVGTFAEIDLGQALNDNIRRCKYVRPTPVQRYAIPISLAGRDLMACAQTGSGKTAAFCFPIISGIMRGPPAQRPQRGGMRTACPLALILSPTRELSMQIHEEARKFSYQTGVRVVVAYGGAPINQQLRDLERGVDILVATPGRLVDLLERARVSLQSIRYLALDEADRMLDMGFEPQVRRIVEQMDMPPPGARQTMLFSATFPKEIQRMASDFLENYIFLAVGRVGSSTDLIVQRVEFVQEADKRSHLMDLLHAQRDSATPGKPTLTLVFVETKRGADSLEHWLCMNGFPATSIHGDRNQQEREYALRSFKSGHTGGSSAYGGGGYGGGAGAPSAWD

SEQ ID NO: 33 - protein, 515 aa, Zea mays
MRSSWADSVANAEESAPATAAANGSVATHSTSRPTRSSYVPPHLRGRSAGAAVEAQAGLVAPAQGGPLPLAAAQPSGQGAAVGGPRWAGIVNGGGGGGSIGAPRQGHGGGGGGRGAWNSRPGGWDRRDREPDPFAKAEAEEIDFDGQENTGINFDAYEDIPVETSGHDVPAPVNTFAEIDLGDALNDNIRRCKYVKPTPVQRYAIPISIAGRDLMACAQTGSGKTAAFCFPIISGILKSPKPHQRSRSTRTACPLALILSPTRELSVQIHEEARKFAYQTGVRVVVAYGGAPITNQLRELERGVEILVATPGRLMDLLERARVSLQMIKYLALDEADRMLDMGFEPQIRKIVEGMDMPQRGERQTMLFSATFPKEIQRMAADFLADYIFLAVGRVGSSTDLIVQRVEFVLDSDKRSYLMDLLHAQKANGTHGKHALTLVFVETKRGADALEDWLFRNGFPATSIHGDRTQQEREHALRSFKSGATGGSSGYGGAYGGGGSGGGGYGGGQSTSSWD

SEQ ID NO: 34 - protein, 514 aa, Zea mays
MRSSWADSVANAEESAPATAAANGSVANHSNSRPTRSSYVPPHLRGRSAGAAVGAQAGSVAPAQGGPLPLAAAQPSGQGAVVGGPRWAGIVNGGGGGSVGAPRQGYGGGGGGGGRGAWNSRPGGWDRRDREPDPFAKAEAEEVDFDGQENTGINFDAYEDIPVETSGHDVPAPVNTFAEIDLGDALNENIRRCKYVKPTPVQRYAIPISIAGRDLMACAQTGSGKTAAFCFPIISGILKSPKPHQRSRSTRTACPLALILSPTRELSVQIHEEARKFAYQTGVRVVVAYGGAPITNQLRELERGVEILVATPGRLMDLLERARVSLQMIKYLALDEADRMLDMGFEPQIRKIVEGMDMPQRGERQTMLFSATFPKEIQRMAADFLADYIFLAVGRVGSSTDLIVQRVEFVLDSDKRSYLMDLIHAQKANGTHGKHALTLVFVETKRGADALEDWLFRNGFPATSIHGDRTQQEREHALRSFKSGATGGSSGYGGAYGGGGSSGYGGGQSTSSWD

SEQ ID NO: 35 - protein, 471 aa, Zea mays
MASSRTSWADVADAEPAPPPPVAAPAAASNGPARSSYVPPHLRNRPPAAAAAAPASAAPPPRSTAGLLTRTGGGSSFGAGGGGGFGGGRARGGARWDREPDPFADSAADAEVPFDEHQNTGINFDAYEDIPVEMSGRDIPPPVSTFADIDLGDALNENIRRCKYVRPTPVQRHAIPISLAGRDLMACAQTGSGKTAAFCFPIISGIMKAPSAGRPQRGGGMGMRTAYPSALILSPTRELSMQIHEEARKFSYQTGVRVVVAYGGAPITQQLRELERGVDILVATPGRLVDLLERARVSLQSIRYLALDEADRMLDMGFEPQVRRIVEQMDMPLPGARQTMLFSATFPKEIQKMASDFLDNYIFLAVGRVGSSTDLIAQRVEFVQEADKRSHLMDLLHAQRDTGKQTLTLVFVETKRGADSLESWLCMNGFPATSIHGDRNQQEREYALRSFKSGQTGGGGHGGAGAPSAWD

SEQ ID NO: 36 - protein, 503 aa, Amborella trichopoda
MAASWADAVANEDLTGKKPNRGSRDSYVPPHLRNKPLSAEAAPYTSPNANPHGASSANVPAKVVGSASPFDNWPGWDMANISPNYGNYGIVDNVRNNRINGWSNNSNRQGSWDHRGREREPYPFEKETRGREREPNPFEKESVDNENTGINFDAYEDIPVETTGENVPPPVNTFAEIDLVEALNQNIRRCNYVKPTPVQRHAIPICLAGRDLMACAQTGSGKTAAFCFPIISGILRSTPSQRPRGARMVYPSALILSPTRELSCQIHQEAKKFAYQTGVKVVVAYGGAPIHHQLRELEKGVDILVATPGRLVDLLERARLSLQMVKYLALDEADRMLDMGFEPQIRKIVNQMDMPPPGMRQTVLFSATFPREIQRLAADFLSNYIFLAVGRVGSSTDLIVQRVEFVQEMDKRSHLMDLLHAQVSNGAQGKQALTLVFVETKRGADSLEHWLCTKGLSATAIHGDRSQYEREQALRSFKSGATYASPGYGVVGPGHAMTSAWGD

SEQ ID NO: 37 - protein, 485 aa, Amborella trichopoda
MRTSWADSVSNSAAENVTAAATNNGPASSASSRPSRSSYVPPHLRNRPPSAEPLAPTPKENLASTNRSAYPGPPPSQPRWGSREPARPGSGPSRGGWGNRTGGWDRGREREVNPFENDEPAEEAFGEQENTGINFDAYEDIPVETSGDNVPPPVNTFAEIDLGDALNENIRRCKYVKPTPVQRHAIPISLGGRDLMACAQTGSGKTAAFCFPIISGIMKGQYAQRPRGARTVYPLALILSPTRELSCQIHDEAKKFSYQTGVRVVVAYGGAPINQQLRELERGVDILVATPGRLVDLLERARVSLQMVKYLALDEADRMLDMGFEPQIRKIVEQMDMPPRGMRQTMLFSATFPKEIQRLASDFLSNYIFLAVGRVGSSTDLIVQRVEYVLESDKRSHLMDLLHAQRANGTHGKQALTLVFVETKKGADSLEHWLCMNGFPATTIHGDRSQQEREQALRSFKSGTTGPSSGYGGGGYGSGVTSAWD

SEQ ID NO: 38 - protein, 329 aa, Arabidopsis thaliana
MAGSAPEGTQFDARQFDQKLNEVLEGQDEFFTSYDDVHESFDAMGLQENLLRGIYAYGFEKPSAIQQRGIVPFCKGLDVIQQAQSGTGKTATFCSGVLQQLDFSLIQCQALVLAPTRELAQQIEKVMRALGDYLGVKVHACVGGTSVREDQRILQAGVHVVVGTPGRVFDMLKRQSLRADNIKMFVLDEADEMLSRGFKDQIYDIFQLLPPKIQVGVFSATMPPEALEITRKFMSKPVRILVKRDELTLEGIKQFYVNVEKEEWKLETLCDLYETLAITQSVIFVNTRRKVDWLTDKMRSRDHTVSATHGDMDQNTRDIIMREFRSGSS

SEQ ID NO: 39 - protein, 329 aa, Arabidopsis thaliana
MAGSAPEGTQFDTRQFDQRLNEVLDGQDEFFTSYDEVHESFDAMGLQENLLRGIYAYGFEKPSAIQQRGIVPFCKGLDVIQQAQSGTGKTATFCSGVLQQLDYALLQCQALVLAPTRELAQQIEKVMRALGDYQGVKVHACVGGTSVREDQRILQAGVHVVVGTPGRVFDMLRRQSLRPDCIKMFVLDEADEMLSRGFKDQIYDIFQLLPPKIQVGVFSATMPPEALEITRKFMSKPVRILVKRDELTLEGIKQFYVNVEKEDWKLETLCDLYETLAITQSVIFVNTRRKVDWLTDKMRSRDHTVSATHGDMDQNTRDIIMREFRSGSS

SEQ ID NO: 40 - protein, 325 aa, Arabidopsis thaliana
MATANPGRGGGRRGGGAMDDDKLVFETTDGIEPITSFNDMGIKEDVLRGVYEYGFEKPSAIQQRAVMPILQGRDVIAQAQSGTGKTSMIALSVCQVVDTSSREVQALILSPTRELATQTEKTIQAIGLHANIQAHACIGGNSVGEDIRKLEHGVHVVSGTPGRVCDMIKRRSLRTRAIKLLILDESDEMLSRGFKDQIYDVYRYLPPDLQVCLVSATLPHEILEMTSKFMTEPVKILVKRDELTLEGIKQFFVAVEKEEWKFDTLCDLYDTLTITQAVIFCNTKRKVDYLSEKMRSHNFTVSSMHGDMPQKERDAIMNEFRSGDS

SEQ ID NO: 41 - DNA, 81 bp, synthetic construct
  source 1..81: mol_type = other DNA
tagtggaataatgaaagatcagcatgtacagagaccccgtgacgtggctcatgtggtcaacttcgatctgccaaatgacat

SEQ ID NO: 42 - DNA, 42 bp, synthetic construct
  source 1..42: mol_type = other DNA
cagcatatagagagaccacgtgagttcggggagtctatcctc

SEQ ID NO: 43 - DNA, 30 bp, synthetic construct
  source 1..30: mol_type = other DNA
atgtagaaagacccggttcacgggcggttt

SEQ ID NO: 44 - DNA, 32 bp, synthetic construct
  source 1..32: mol_type = other DNA
tggtcttgacattcccccacgtggctcatgtt

SEQ ID NO: 45 - DNA, 24 bp, synthetic construct
  source 1..24: mol_type = other DNA
agagaccacgtgagttcggggagt

SEQ ID NO: 46 - DNA, 80 bp, synthetic construct
  source 1..80: mol_type = other DNA
gtggggtgagacagacaatgctgttcagtgctacatttccaagtcatgttgtaaacttcgatctaccaaatgacattgat

SEQ ID NO: 47 - DNA, 36 bp, synthetic construct
  source 1..36: mol_type = other DNA
catatagagagaccacgtgggagttcggggagtcta

SEQ ID NO: 48 - DNA, 483 bp, synthetic construct
  source 1..483: mol_type = other DNA
tctagaaacagcatccgtttttataatttaattttcttacaaaggtaggaccaacatttgtgatctataaatcttcctactacgttatatagagacccttcgacataacacttaactcgtttatatatttgttttacttgttttgcacatacacacaaaaataaaaaagactttatatttatttactttttaatcacacggattagctccggcgaagtatggtcgtcgtcttcatcttcttcctccatcatcagatttttccttaaatggaagaaaccaaacgaaactccgatcttctccgttctcgtgttttcctctctggcttttattgctgggattgggaatttctcaccgctctcttgctttttagttgctgattctttttccttcgactttctatttccaatctttcttcttctctttgtgtattagattatttttagttttatttttctgtggtaaaataaaaaaagttcgccggag

SEQ ID NO: 49 - DNA, 483 bp, synthetic construct
  source 1..483: mol_type = other DNA
tctagaaacagcatccgtttttataatttaattttcttacaaaggtaggaccaacatttgtgatctataaatcttcctactacgttatatagagacccttcgacataacacttaactcgtttatatatttgttttacttgttttgcacatacacacaaaaataaaaaagactttatatttatttactttttaatcacacggattagctccggcgaagtatggtcgtcgtcttcatcttcttcctccatcatcagatttttccttaaatggaagaaaccaaagtaaactttgatgttattcattctcgtgttttcctctctggcttttattgctgggattgggaatttctcaccgctctcttgctttttagttgctgattctttttccttcgactttctatttccaatctttcttcttctctttgtgtattagattatttttagttttatttttctgtggtaaaataaaaaaagttcgccggag

SEQ ID NO: 50 - DNA, 125 bp, synthetic construct
  source 1..125: mol_type = other DNA
ctcctcttcttctctctctcataaaacaaaagatcctctctgcttctcctttccctttttcaaaatctctctctctctctctctttttagatccagttttagggttttcttctctgtgagcgaag

SEQ ID NO: 51 - DNA, 125 bp, synthetic construct
  source 1..125: mol_type = other DNA
ctcctcttcttctctctctcataaaacaaaagatggtctctgcttctcctttccctttttcaaaatctctctctctctctctctttttagatccagttttagggttttcttctctgtgagcgaag

SEQ ID NO: 52 - DNA, 125 bp, synthetic construct
  source 1..125: mol_type = other DNA
ctcctcttcttctctctctcataaaacttcctatgctctctgcttctcctttccctttttcaaaatctctctctctctctctctttttagatccagttttagggttttcttctctgtgagcgaag

SEQ ID NO: 53 - DNA, 125 bp, synthetic construct
  source 1..125: mol_type = other DNA
ctcctcttcttctctctctcataaaacttcctatgctctctgcttctgacgtcggttccgacgtctctctctctctctctctctttttagatccagttttagggttttcttctctgtgagcgaag

SEQ ID NO: 54 - DNA, 125 bp, synthetic construct
  source 1..125: mol_type = other DNA
ctcctcttcttctctctctcataaaacttcctatgctctctgcttctcccgtcggttccgacgaatctctctctctctctctctttttagatccagttttagggttttcttctctgtgagcgaag

SEQ ID NO: 55 - DNA, 125 bp, synthetic construct
  source 1..125: mol_type = other DNA
ctcctcttcttctctctctcataaaacttcctatgctctctgcttctcctttcggttccgaaaaatctctctctctctctctctttttagatccagttttagggttttcttctctgtgagcgaag

SEQ ID NO: 56 - DNA, 125 bp, synthetic construct
  source 1..125: mol_type = other DNA
ctcctcttcttctctctctcataaaacaaaagatggtctctgcttctgacgtcggttccgacgtctctctctctctctctctctttttagatccagttttagggttttcttctctgtgagcgaag

SEQ ID NO: 57 - DNA, 125 bp, synthetic construct
  source 1..125: mol_type = other DNA
ctcctcttcttctctctctcataaaacaaaagatcctctctgcttctgacgtcggttccgacgtctctctctctctctctctctttttagatccagttttagggttttcttctctgtgagcgaag

SEQ ID NO: 58 - DNA, 282 bp, synthetic construct
  source 1..282: mol_type = other DNA
catttctactttgcccgcccacagatgtagttttctctgcgcgtgtgcgttttccctcctccccgccctcagggtccacggccaccatggcgtattaggggcagcagtgcctgcggcagcattggcctttgcagcggcggcagcagcaccaggctctgcagcggcaacccccagcggcttaagccatggcgcttctcacggcattcagcagcagcgttgctgtaaccgacaaagacaccttcgaattaagcacattcctcgattccagcaaagcaccgcaac

SEQ ID NO: 59 - DNA, 282 bp, synthetic construct
  source 1..282: mol_type = other DNA
catttctactttgcccgcccacagatgtagttttctctgcgcgtgtgcgttttccctcctccccgccctcagggtccacggccaccatggcgtattaggggcagcagtgcctgcggcagcattggcctttgcagcggcggcagcagcaccaggctctgcagcggcaacccccagcggcttaagccaaggcgcttctcacggcattcagcagcagcgttgctgtaaccgacaaagacaccttcgaattaagcacattcctcgattccagcaaagcaccgcaac

SEQ ID NO: 60 - DNA, 282 bp, synthetic construct
  source 1..282: mol_type = other DNA
catttctactttgcccgcccacagatgtagttttctctgcgcgtgtgcgttttccctcctccccgccctcagggtccacggccaccatggcgtattaggggcagcagtgcctgcggcagcattggcctttgcagcggcggcagcagcaccaggctctgcagcggcaacccccagcggcttaagccatggcgcttctcacggacgtcggttccgacgtcgctgtaaccgacaaagacaccttcgaattaagcacattcctcgattccagcaaagcaccgcaac

SEQ ID NO: 61 - DNA, 282 bp, synthetic construct
  source 1..282: mol_type = other DNA
catttctactttgcccgcccacagatgtagttttctctgcgcgtgtgcgttttccctcctccccgccctcagggtccacggccaccatggcgtattaggggcagcagtgcctgcggcagcattggcctttgcagcggcggcagcagcaccaggctctgcagcggcaacccccagcggcttaagccaaggcgcttctcacggacgtcggttccgacgtcgctgtaaccgacaaagacaccttcgaattaagcacattcctcgattccagcaaagcaccgcaac

SEQ ID NO: 62 - DNA, 379 bp, synthetic construct
  source 1..379: mol_type = other DNA
gggcagtttgtaggtcgcgagggaagcgctgaggatcaggaagggggcactgagtgtccgtgggggaatcctcgtgataggaactggaatatgccttgagggggacactatgtctttaaaaacgtcggctggtcatgaggtcaggagttccagaccagcctgaccaacgtggtgaaactccgtctctactaaaaatacaaaaattagccgggcgtggtgccgctccagctactcaggaggctgaggcaggagaatcgctagaacccgggaggcggaggttgcagtgagccgagatcgcgccattgcactccagcctgggcgacagagcgagactgtctcaaaacaaaacaaaacaaaacaaaacaaaaaacaccggctg

SEQ ID NO: 63 - DNA, 379 bp, synthetic construct
  source 1..379: mol_type = other DNA
gggcagtttgtaggtcgcgagggaagcgctgaggatcaggaagggggcactgagtgtccgtgggggaatcctcgtgataggaactggaatatgccttgagggggacactaagtctttaaaaacgtcggctggtcatgaggtcaggagttccagaccagcctgaccaacgtggtgaaactccgtctctactaaaaatacaaaaattagccgggcgtggtgccgctccagctactcaggaggctgaggcaggagaatcgctagaacccgggaggcggaggttgcagtgagccgagatcgcgccattgcactccagcctgggcgacagagcgagactgtctcaaaacaaaacaaaacaaaacaaaacaaaaaacaccggctg

SEQ ID NO: 64 - DNA, 379 bp, synthetic construct
  source 1..379: mol_type = other DNA
gggcagtttgtaggtcgcgagggaagcgctgaggatcaggaagggggcactgagtgtccgtgggggaatcctcgtgataggaactggaatatgccttgagggggacactatgtctttaaaaacgtcggctggtcaagaggtcaggagttccagaccagcctgaccaacgtggtgaaactccgtctctactaaaaatacaaaaattagccgggcgtggtgccgctccagctactcaggaggctgaggcaggagaatcgctagaacccgggaggcggaggttgcagtgagccgagatcgcgccattgcactccagcctgggcgacagagcgagactgtctcaaaacaaaacaaaacaaaacaaaacaaaaaacaccggctg

SEQ ID NO: 65 - DNA, 379 bp, synthetic construct
  source 1..379: mol_type = other DNA
gggcagtttgtaggtcgcgagggaagcgctgaggatcaggaagggggcactgagtgtccgtgggggaatcctcgtgataggaactggaatatgccttgagggggacactaagtctttaaaaacgtcggctggtcaagaggtcaggagttccagaccagcctgaccaacgtggtgaaactccgtctctactaaaaatacaaaaattagccgggcgtggtgccgctccagctactcaggaggctgaggcaggagaatcgctagaacccgggaggcggaggttgcagtgagccgagatcgcgccattgcactccagcctgggcgacagagcgagactgtctcaaaacaaaacaaaacaaaacaaaacaaaaaacaccggctg

SEQ ID NO: 66 - DNA, 125 bp, synthetic construct; identical to SEQ ID NO: 50
  source 1..125: mol_type = other DNA

SEQ ID NO: 67 - DNA, 125 bp, synthetic construct
  source 1..125: mol_type = other DNA
ctcctcttcttctctctctcataaaacaaaagatcctctctgcttctcctttcggttccgaaaaatctctctctctctctctctttttagatccagttttagggttttcttctctgtgagcgaag

SEQ ID NO: 68 - DNA, 125 bp, synthetic construct; identical to SEQ ID NO: 57
  source 1..125: mol_type = other DNA

SEQ ID NO: 69 - DNA, 125 bp, synthetic construct
  source 1..125: mol_type = other DNA
ctcctcttcttctctctctcataaaacaaaagatcctctctgcgtgggacgtcggttccgacgtcccactctctctctctctctttttagatccagttttagggttttcttctctgtgagcgaag

SEQ ID NO: 70 - DNA, 125 bp, synthetic construct
  source 1..125: mol_type = other DNA
ctcctcttcttctctctctcataaaacaaaagatcctctgagcgtgggacgtcggttccgacgtcccacgctctctctctctctttttagatccagttttagggttttcttctctgtgagcgaag

SEQ ID NO: 71 - DNA, 125 bp, synthetic construct
  source 1..125: mol_type = other DNA
ctcctcttcttctctctctcataaaacaaaagatcctccgagcgtgggacgtcggttccgacgtcccacgctcggagtctctctttttagatccagttttagggttttcttctctgtgagcgaag

SEQ ID NO: 72 - DNA, 125 bp, synthetic construct
  source 1..125: mol_type = other DNA
ctcctcttcttctctctctcataaaacaaaagaccctccgagcgtgggacgtcggttccgacgtcccacgctcggagggtctctttttagatccagttttagggttttcttctctgtgagcgaag

SEQ ID NO: 73 - DNA, 18 bp, synthetic construct
  source 1..18: mol_type = other DNA
gacgtcggttccgacgtc

SEQ ID NO: 74 - RNA, 101 nt, synthetic construct
  source 1..101: mol_type = other RNA
gagatagaagcaagcagacaacaaaccaccgtaccggtttcagtcggcggtgggaattttccggttggtgggttaagtccgttgagtgaagctatatggag

SEQ ID NO: 75 - RNA, 101 nt, synthetic construct
  source 1..101: mol_type = other RNA
aaatgggagaaatggagatcgaagaaatcgaagctgttcttgagaaaatctgggatctacatgacaagcttagcgatgagattcacttgatttcgaagtct

SEQ ID NO: 76 - RNA, 101 nt, synthetic construct
  source 1..101: mol_type = other RNA
agttcttcagagagtgtggaaaacgagtgcatgtgttgggctgcaagagatccatctggtcttctttctcctcatactatcactcgcaggtctgttacaac

SEQ ID NO: 77 - RNA, 101 nt, synthetic construct
  source 1..101: mol_type = other RNA
tgttagagatatccggagatctcaaccgttggattctttctccttcaaatcaaattataaatcccatcaaacagagagagagagagaagaggaggagaatt

SEQ ID NO: 78 - RNA, 101 nt, synthetic construct
  source 1..101: mol_type = other RNA
gagagtatctggcgaatcgcgacgggacaagatccgagccgtgaagattacgaagggatcgagttctggtcaaaccctgagcgttctggttggctcacaaa

SEQ ID NO: 79 - RNA, 101 nt, synthetic construct
  source 1..101: mol_type = other RNA
ctaaattgagtttcgtcgtcttctgcttctgcttaagcttcttcatcatttagctccagaggcggagctttcttctctctctgtggacgaccatggcggcg

SEQ ID NO: 80 - RNA, 101 nt, synthetic construct
  source 1..101: mol_type = other RNA
caaagctcaatgacaatggaactacgaccatcaggtgattccggttcatctgacgtcgatgctgagatcagcgatggcttttcaccgctcgatacttctca

SEQ ID NO: 81 - RNA, 101 nt, synthetic construct
  source 1..101: mol_type = other RNA
gcgttgctgaagtctttcatcgatgttggctcagactcgcacttccctatccagaatctcccttatggtgtcttcaaaccggaatcgaactcaactcctcg

SEQ ID NO: 82 - RNA, 101 nt, synthetic construct
  source 1..101: mol_type = other RNA
cagcttcaggaaatccaagataacataaggagtagacgcaataagatatttctcctcatggaggaagtgaggaggttacgtgtgcagcaacgtatcaagag

SEQ ID NO: 83 - RNA, 101 nt, synthetic construct
  source 1..101: mol_type = other RNA
tatccttctctcgacgatgatttcgtctctgatttgttttgcttcgatcaaagcaatggagcagaacttgatgattacacacagtttggtgtaaatttgca

SEQ ID NO: 84 - RNA, 101 nt, synthetic construct
  source 1..101: mol_type = other RNA
aaattggattacgatggctggatatcctactaatggatcagtctacgtttcaaatctccccttaggaactgacgagaatatgttggctgactattttggga

SEQ ID NO: 85 - RNA, 101 nt, synthetic construct
  source 1..101: mol_type = other RNA
gagcgtctaacatctcctcctcgtttgatgattgtctctgatcttgatcatactatggttgatcatcatgatcctgagaatctatctctgctgagattcaa

SEQ ID NO: 86 - RNA, 101 nt, synthetic construct
  source 1..101: mol_type = other RNA
tgagaagaaaatgtcaggatctgagacgggtttaatggcggcgaccagagaatcaatgcaatttacaatggctctccaccagcagcagcaacacagtcaag

SEQ ID NO: 87 - RNA, 101 nt, synthetic construct
  source 1..101: mol_type = other RNA
tcgacggtgtacgtgctagagccgccgacaaagggaaaggtcattgtaaatactactcatggtccaatcgacgtcgagctttggcctaaggaagcgcccaa

SEQ ID NO: 88 - RNA, 101 nt, synthetic construct
  source 1..101: mol_type = other RNA
tcggcggagagttgtttcggaagctcgggtgatcaaagcagcagcaaaggagtggctactcatggtggtagctatgttcagtataatgtctatggcaatct

SEQ ID NO: 89 - RNA, 101 nt, synthetic construct
  source 1..101: mol_type = other RNA
gaaagcttggacactaattttcctgtgcgccatagaaaggtctcgtttgaaagtaagggaaacaagacagagattgtgatctgcagctatgaagatcatat

SEQ ID NO: 90 - RNA, 101 nt, synthetic construct
  source 1..101: mol_type = other RNA
ggtttcttctcttttcttggaagagttctcttcgcttctttattcatcctctccgcttggcaaatgttcaatgactttggaactgacggtggtccagcagc

SEQ ID NO: 91 - RNA, 101 nt, synthetic construct
  source 1..101: mol_type = other RNA
aatactggtggtcggctcatcgctggttctcacaataggaatgagttcgtgttgattaatgcagacgagagtgccagaattagatcagtggaagaactaag

SEQ ID NO: 92 - RNA, 101 nt, synthetic construct
  source 1..101: mol_type = other RNA
gcttctgttatctcttcctctccttttctatgcaaatcatcctccaagagtgatttggggatttcttcgtttcctaaatcttctcagatttcgattcatcg

SEQ ID NO: 93 - RNA, 101 nt, synthetic construct
  source 1..101: mol_type = other RNA
agtctcttcaacactgaaaacacatgggcctttgtctttggcttgctcggcaaccttatctcctttgccgtgttcctatctcctgtgccaacgttctatag